(19) United States
(12) Patent Application Publication
(10) Pub. No.: US 2018/0042929 A1
(43) Pub. Date: Feb. 15, 2018

(54) USE OF SUBSTITUTED 2,3-DIHYDROIMIDAZO[1,2-C]QUINAZOLINES

(71) Applicants: Bayer Pharma Aktiengesellschaft, Berlin (DE); Bayer HealthCare Pharmaceuticals Inc., Whippany, NJ (US)

(72) Inventors: Ningshu LIU, Berlin (DE); Carol PENA, Basking Ridge, NJ (US); Michael JEFFERS, Ridgewood, NJ (US); Isabelle GENVRESSE, Berlin (DE)

(73) Assignees: Bayer Pharma Aktiengesellschaft, Berlin (DE); Bayer HealthCare Pharmaceuticals Inc., Whippany, NJ (US)

(21) Appl. No.: 15/557,036

(22) PCT Filed: Mar. 7, 2016
(86) PCT No.: PCT/EP2016/054728

§ 371 Date: Sep. 8, 2017

Related U.S. Application Data
(60) Provisional application No. 62/130,547, filed on Mar. 9, 2015.

(51) Int. Cl. A61K 31/519; C12Q 1/68; A61K 31/5377

(52) U.S. Cl. CPC A61K 31/519; A61K 31/5377; C12Q 1/6886; A61K 2300/00; C12Q 2600/158; C12Q 2600/106

(57) ABSTRACT

The present invention relates to: use of a 2,3-dihydroimidazo[1,2-c]quinazoline compound, or of a pharmaceutical composition containing same, as a sole active agent, or of a combination of a) said compound or a pharmaceutical composition containing said compound and b) one or more further active agents, for the preparation of a medicament for the treatment or prophylaxis of endometrial cancer (hereinafter abbreviated to “EC”), particularly 1st line, 2nd line, relapsed, refractory, type I or type II EC, or endometriosis; combinations of a) said compound and b) one or more further active agents; a pharmaceutical composition comprising said compound as a sole active agent for the treatment of endometrial cancer (hereinafter abbreviated to “EC”), particularly 1st line, 2nd line, relapsed, refractory, type I or type II EC, or endometriosis; a pharmaceutical composition comprising a combination of a) said compound and b) one or more further active agents; use of biomarkers which is the loss of tumor suppressor PTEN or FBXW7, for predicting the sensitivity and/or resistance of a cancer patient to said compound and providing a rationale-based dosage to increase sensitivity and/or to overcome resistance; a method of determining the loss of tumor suppressor PTEN or FBXW7; and a method for determining perturbations in PIK3CA, PIK3CB, PIK3CD, PIK3CG, P1K3R1, PIK3R2, PIK3R3, PIK3R4, PIK3R5, FGFR1, FGFR2, FGFR3 and/or FGFR4.

The present invention relates to:
use of a 2,3-dihydroimidazo[1,2-c]quinazoline compound, or of a pharmaceutical composition containing same, as a sole active agent, or of a combination of a) said compound or a pharmaceutical composition containing said compound and b) one or more further active agents, for the preparation of a medicament for the treatment or prophylaxis of cancer, particularly endometrial cancer (hereinafter abbreviated to “EC”), particularly 1st line, 2nd line, relapsed, refractory, type I or type II EC, or endometriosis; as a single agent or in combination with one or more other active agents;
combinations of a) said compound and b) one or more further active agents;
a pharmaceutical composition comprising said compound as a sole active agent for the treatment of cancer, particularly endometrial cancer (hereinafter abbreviated to “EC”), particularly 1st line, 2nd line, relapsed, refractory, type I or type II EC, or endometriosis;
a pharmaceutical composition comprising a combination of a) said compound and b) one or more further active agents;
use of biomarkers, such as the loss of tumor suppressor PTEN or FBXW7, either alone or in combination with another form of PI3K pathway activation selected from perturbation of any of the following alone or in combination: mutation in PIK3CA, PIK3CB, PIK3CD, PIK3CG, PIK3R1, PIK3R2, PIK3R3, PIK3R4, PIK3R5, FGFR1, FGFR2, FGFR3 and/or FGFR4; PTEN-loss and alteration of PIK3CA, PIK3CB, PIK3CD, PIK3CG, PIK3R1, PIK3R2, PIK3R3, PIK3R4, PIK3R5, FGFR1, FGFR2, FGFR3 and/or FGFR4 which may be measured at either the protein level, mRNA level, or DNA level,

for predicting the sensitivity and/or resistance of a patient with endometrial cancer (hereinafter abbreviated to “EC”), particularly 1st line, 2nd line, relapsed, refractory, type I or type II EC, or endometriosis, to a 2,3-dihydroimidazo[1,2-c]quinazoline compound as defined herein, thus providing rationale-based dosage as defined herein to overcome said resistance of a patient with endometrial cancer (hereinafter abbreviated to “EC”), particularly 1st line, 2nd line, relapsed, refractory, type I or type II EC, or endometriosis, to a 2,3-dihydroimidazo[1,2-c]quinazoline compound as defined herein (patient stratification);
a method of determining the loss of tumor suppressor PTEN or FBXW7; and
a method for determining perturbations in PIK3CA, PIK3CB, PIK3CD, PIK3CG, PIK3R1, PIK3R2, PIK3R3, PIK3R4, PIK3R5, FGFR1, FGFR2, FGFR3 and/or FGFR4. PTEN loss and alteration of PIK3CA, PIK3CB, PIK3CD, PIK3CG, PIK3R1, PIK3R2, PIK3R3, PIK3R4, PIK3R5, FGFR1, FGFR2, FGFR3 and/or FGFR4.

### BACKGROUND OF THE INVENTION

Cancer is a complex disease arising after a selection process for cells with acquired functional capabilities like enhanced survival/resistance towards apoptosis and a limitless proliferative potential. Thus, it is preferred to develop drugs for cancer therapy addressing distinct features of established tumors.

The PI3K signaling pathway is one of the prominent pathways that promote tumor cell survival. PI3K is activated by many cancer related receptor tyrosine kinases (e.g. PDGFR, EGFR, HER2/3, or IGF-1R), cell adhesion molecules, GPCR, and oncogenic proteins (such as Ras). The PI3K pathway activation by genetic alteration of PI3K (activation mutation and/or amplification) and/or loss-of-function of the tumor suppressor PTEN are frequently found in many tumors. Furthermore, activation of PI3K is one of the major mechanisms causing the resistance of tumors to radio-, chemo- and targeted therapeutics.

Once PI3K is activated, it catalyzes the generation of PIP3 from PIP2. The biological active PIP3 binds to the pleckstrin homology (PH) domains of PDK-1, AKT, and other PH-domain containing proteins, such as Rho and PLC. As the consequence of binding to PIP3, these proteins are translocated to the cell membrane and are subsequently activated to induce tumor cell proliferation, survival, invation and migration.

Fibroblast growth factors (FGFs) and their receptors (FGFRs) drive crucial developmental signaling pathways, which are responsible for many functions of the tumor cells, including cell proliferation, survival and migration through downstream signalling pathways mediated by PLCγ/PKC, RAS/MAPK, PI3K/AKT, and STATs. FGFR signalling pathways also regulate tumor stromal cells as well as tumor angiogenesis. There are several types of genetic evidence that support an oncogenic function of FGFRs: gene amplifications, activating mutations, chromosomal translocations and aberrant splicing at the post-transcriptional level.

Endometrial cancer (EC) is the most common gynecologic malignancy in industrialized countries, with an incidence rate of 12.9 per 100,000 women per year. Early-stage EC (stage I or II) can be effectively treated with surgery, while treatment of recurrent or high grade metastatic disease is limited to cytotoxic chemotherapy, e.g. paclitaxel and carboplatin. In addition, for recurrent EC, there are still no agreement and no definitive drugs of choice in spite of the poor prognosis of this subset of patients. It is noteworthy that the available chemotherapies do not provide long-term disease control, and many patients demonstrate intrinsic resistance and significant toxicities to these therapies. As such, it remains an important unmet medical need for recurrent EC. The successful management of these patients depends on the identification and understanding of molecular mechanisms underlying the initiation and progression of EC to achieve a more tailored therapy, based on the biological tumor profile.

As described in the present text, the anti-tumor efficacy of the PI3K inhibitor copanlisib was investigated in preclinical tumor models in vitro and in vivo as single agent and in combination. It was found that the PI3K inhibitor copanlisib showed potent anti-tumor activity in a subset of endometrial tumors models with activated PI3K pathway. The activity correlated with PIK3CA activating mutation, loss-of-function of PTEN, activation of RTKs and KRAS mutation status. Copanlisib also showed clinical benefit as single agent in the advanced metastatic endometrial cancer in the first-in-man study in clinic, including a complete response in a patient with PIK3CA mutation and loss of PTEN expression.

The present invention is thus to identify molecular markers predicting the sensitivity and/or resistance of the cancer patients toward the PI3K inhibitors described herein. Furthermore, the present invention also relates to the identification of resistance mechanisms and therefore provides a rationale-based dosage to overcome the resistance.

To the Applicant's knowledge, no specific disclosure in the prior art is known that 2,3-dihydroimidazo[1,2-c]quinazoline compounds would be effective in the treatment or prophylaxis of endometrial cancer (hereinafter abbreviated to “EC”), particularly 1st line, 2nd line, relapsed, refractory, type I or type II EC, or endometriosis.

It has been found, and this is the basis of the present invention, that 2,3-dihydroimidazo[1,2-c]quinazoline compounds, as described and defined herein, show a beneficial effect in the treatment or prophylaxis of endometrial cancer (hereinafter abbreviated to “EC”), particularly 1st line, 2nd line, relapsed, refractory, type I or type II EC, or endometriosis.

Thus, in accordance with a first aspect, the present invention relates to the use of 2,3-dihydroimidazo[1,2-c]quinazoline compounds, or a physiologically acceptable salt, solvate, hydrate or stereoisomer thereof, as a sole active agent, or of pharmaceutical compositions containing such compounds or a physiologically acceptable salt, solvate, hydrate or stereoisomer thereof, for the preparation of a medicament for the treatment or prophylaxis of endometrial cancer (hereinafter abbreviated to “EC”), particularly 1st line, 2nd line, relapsed, refractory, type I or type II EC, or endometriosis.

In accordance with a second aspect, the present invention relates to combinations of:

a) a 2,3-dihydroimidazo[1,2-c]quinazoline compound, or a physiologically acceptable salt, solvate, hydrate or stereoisomer thereof; and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent.

In accordance with a third aspect, the present invention relates to pharmaceutical compositions comprising a 2,3-dihydroimidazo[1,2-c]quinazoline compound, or a physiologically acceptable salt, solvate, hydrate or stereoisomer thereof, as a sole active agent, for the treatment of endometrial cancer (hereinafter abbreviated to “EC”), particularly 1st line, 2nd line, relapsed, refractory, type I or type II EC, or endometriosis.

In accordance with a fourth aspect, the present invention relates to pharmaceutical compositions comprising a combination of:

a) a 2,3-dihydroimidazo[1,2-c]quinazoline compound, or a physiologically acceptable salt, solvate, hydrate or stereoisomer thereof; and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent.

In accordance with a fifth aspect, the present invention relates to the use of combinations of:

a) a 2,3-dihydroimidazo[1,2-c]quinazoline compound, or a physiologically acceptable salt, solvate, hydrate or stereoisomer thereof;

or of a pharmaceutical composition containing such a compound or a physiologically acceptable salt, solvate, hydrate or stereoisomer thereof,

and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent;

for the preparation of a medicament for the treatment or prophylaxis of endometrial cancer (hereinafter abbreviated to “EC”), particularly 1st line, 2nd line, relapsed, refractory, type I or type II EC, or endometriosis.

In accordance with a sixth aspect, the present invention relates to use of biomarkers, such as the loss of tumor suppressor PTEN or FBXW7,
either alone or in combination with another form of PI3K pathway activation selected from perturbation of any of the following alone or in combination: mutation in PIK3CA, PIK3CB, PIK3CD, PIK3CG, PIK3R1, PIK3R2, PIK3R3, PIK3R4, PIK3R5, FGFR1, FGFR2, FGFR3 and/or FGFR4; PTEN-loss and alteration of PIK3CA, PIK3CB, PIK3CD, PIK3CG, PIK3R1, PIK3R2, PIK3R3, PIK3R4, PIK3R5, FGFR1, FGFR2, FGFR3 and/or FGFR4, which may be measured at either the protein level, mRNA level, or DNA level,

for predicting the sensitivity and/or resistance of a patient with endometrial cancer (hereinafter abbreviated to “EC”), particularly 1st line, 2nd line, relapsed, refractory, type I or type II EC, or endometriosis, to a 2,3-dihydroimidazo[1,2-c]quinazoline compound as defined herein, thus providing rationale-based dosage as defined herein to overcome said resistance of a patient with endometrial cancer (hereinafter abbreviated to “EC”), particularly 1st line, 2nd line, relapsed, refractory, type I or type II EC, or endometriosis, to a 2,3-dihydroimidazo[1,2-c]quinazoline compound as defined herein (patient stratification).

In accordance with a seventh aspect, the present invention relates to a method of determining the loss of tumor suppressor PTEN or FBXW7.

In accordance with an eighth aspect, the present invention relates to a method for determining perturbations in PIK3CA, PIK3CB, PIK3CD, PIK3CG, PIK3R1, PIK3R2, PIK3R3, PIK3R4, PIK3R5, FGFR1, FGFR2, FGFR3 and/or FGFR4. PTEN loss and alteration of PIK3CA, PIK3CB, PIK3CD, PIK3CG, PIK3R1, PIK3R2, PIK3R3, PIK3R4, PIK3R5, FGFR1, FGFR2, FGFR3 and/or FGFR4.

In accordance a particular embodiment of any of the above aspects of the present invention, said cancer is endometrial cancer (hereinafter abbreviated to “EC”), particularly 1st line, 2nd line, relapsed, refractory, type I or type II EC, or endometriosis.

In accordance a particular embodiment of any of the above aspects of the present invention, said cancer is 1st line, 2nd line, relapsed, refractory, type I EC.

In accordance a particular embodiment of any of the above aspects of the present invention, said cancer is 1st line, 2nd line, relapsed, refractory, type II EC, or endometriosis.

### DETAILED DESCRIPTION OF THE INVENTION

A first aspect of the present invention relates to the use of a compound of general formula (A):

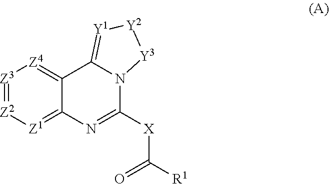

in which:

X represents CR⁵R⁶or NH;

Y¹ represents CR³ or N;

the chemical bond between Y² [symbol] Y³ represents a single bond or double bond,

with the proviso that when the Y² [symbol] Y³ represents a double bond, Y² and Y³ independently represent CR⁴ or N, and

when Y² [symbol] Y³ represents a single bond, Y² and Y³ independently represent CR³R⁴ or NR⁴;

Z¹, Z², Z³ and Z⁴ independently represent CH , CR² or N;
R¹ represents aryl optionally having 1 to 3 substituents selected from R¹¹, C₃₋₈ cycloalkyl optionally having 1 to 3 substituents selected from R¹¹,
   C₁₋₆ alkyl optionally substituted by aryl, heteroaryl, C₁₋₆ alkoxyaryl, aryloxy, heteroaryloxy or one or more halogen,
   C₁₋₆ alkoxy optionally substituted by carboxy, aryl, heteroaryl, C₁₋₆ alkoxyaryl, aryloxy, heteroaryloxy or one or more halogen,
   or
   a 3 to 15 membered mono- or bi-cyclic heterocyclic ring that is saturated or unsaturated, optionally having 1 to 3 substituents selected from R¹¹, and contains 1 to 3 heteroatoms selected from the group consisting of N, O and S,
   wherein
   R¹¹ represents halogen, nitro, hydroxy, cyano, carboxy, amino, N-(C₁₋₆alkyl)amino, N-(hydroxyC₁₋₆alkyl)amino, N,N-di(C₁₋₆alkyl)amino, N-(C₁₋₆acyl)amino, N-(formyl)-N-(C₁₋₆alkyl)amino, N-(C₁₋₆alkanesulfonyl) amino, N-(carboxyC₁₋₆alkyl)-N-(C₁₋₆alkyl)amino, N-(C₁₋₆alkoxycabonyl)amino, N-[N,N-di(C₁₋₆alkyl)amino methylene]amino, N-[N,N-di(C₁₋₆alkyl)amino (C₁₋₆alkyl)methylene]amino, N-[N,N-di(C₁₋₆alkyl)amino C₂₋₆alkenyl]amino, aminocarbonyl, N-(C₁₋₆alkyl)aminocarbonyl, N,N-di(C₁₋₆alkyl)aminocarbonyl, C₃₋₈cycloalkyl, C₁₋₆ alkylthio, C₁₋₆alkanesulfonyl, sulfamoyl, C₁₋₆alkoxycarbonyl,
   N-arylamino wherein said aryl moiety is optionally having 1 to 3 substituents selected from R¹⁰¹, N-(aryl C₁₋₆alkyl)amino wherein said aryl moiety is optionally having 1 to 3 substituents selected from R¹⁰¹, aryl C₁₋₆alkoxycarbonyl wherein said aryl moiety is optionally having 1 to 3 substituents selected from R¹⁰¹,
   C₁₋₆alkyl optionally substituted by mono-, di- or tri-halogen, amino, N-(C₁₋₆alkyl)amino or N,N-di(C₁₋₆alkyl)amino,
   C₁₋₆alkoxy optionally substituted by mono-, di- or tri-halogen, N-(C₁₋₆alkyl)sulfonamide, or N-(aryl)sulfonamide,
   or
   a 5 to 7 membered saturated or unsaturated ring having 1 to 3 heteroatoms selected from the group consisting of O, S and N, and optionally having 1 to 3 substituents selected from R¹⁰¹
   wherein
   R¹⁰¹ represents halogen, carboxy, amino, N-(C₁₋₆ alkyl)amino, N,N-di(C₁₋₆alkyl)amino, aminocarbonyl, N-(C₁₋₆alkyl)aminocarbonyl, N,N-di(C₁₋₆alkyl)aminocarbonyl, pyridyl,
      C₁₋₆ alkyl optionally substituted by cyano or mono- di- or tri-halogen,
      and
      C₁₋₆alkoxy optionally substituted by cyano, carboxy, amino, N-(C₁₋₆ alkyl)amino, N,N-di(C₁₋₆alkyl)amino, aminocarbonyl, N-(C₁₋₆alkyl)aminocarbonyl, N,N-di(C₁₋₆alkyl)aminocarbonyl or mono-, di- or tri-halogen;
R² represents hydroxy, halogen, nitro, cyano, amino, N-(C₁₋₆alkyl)amino, N,N-di(C₁₋₆alkyl)amino, N-(hydroxyC₁₋₆alkyl)amino, N-(hydroxyC₁₋₆alkyl)-N-(C₁₋₆alkyl)amino, C₁₋₆ acyloxy, aminoC₁₋₆ acyloxy, C₂₋₆alkenyl, aryl,
   a 5-7 membered saturated or unsaturated heterocyclic ring having 1 to 3 heteroatoms selected from the group consisting O, S and N, and optionally substituted by
   hydroxy, C₁₋₆ alkyl, C₁₋₆ alkoxy, oxo, amino, amino C₁₋₆alkyl, N-(C₁₋₆alkyl)amino, N,N-di(C₁₋₆alkyl)amino, N-(C₁₋₆ acyl)amino, N-(C₁₋₆alkyl)carbonylamino, phenyl, phenyl C₁₋₆ alkyl, carboxy, C₁₋₆alkoxycarbonyl, aminocarbonyl, N-(C₁₋₆alkyl)aminocarbonyl, or N,N-di(C₁₋₆alkyl)amino, —C(O)— R²⁰
   wherein
   R²⁰ represents C₁₋₆ alkyl, C₁₋₆ alkoxy, amino, N-(C₁₋₆alkyl)amino, N,N-di(C₁₋₆alkyl)amino, N-(C₁₋₆ acyl)amino, or a 5-7 membered saturated or unsaturated heterocyclic ring having 1 to 3 heteroatoms selected from the group consisting O, S and N, and optionally substituted by C₁₋₆ alkyl, C₁₋₆ alkoxy, oxo, amino, N-(C₁₋₆alkyl)amino, N,N-di(C₁₋₆alkyl)amino, N-(C₁₋₆ acyl)amino, phenyl, or benzyl,
      C₁₋₆ alkyl optionally substituted by R²¹,
      or
      C₁₋₆ alkoxy optionally substituted by R²¹,
      wherein
      R²¹ represents cyano, mono-, di or tri-halogen, hydroxy, amino, N-(C₁₋₆alkyl)amino, N,N-di(C₁₋₆alkyl)amino, N-(hydroxyC₁₋₆ alkyl) amino, N-(halophenylC₁₋₆ alkyl) amino, amino C₂₋₆ alkylenyl, C₁₋₆ alkoxy, hydroxyC₁₋₆ alkoxy, —C(O)— R²⁰¹, —NHC(O)— R²⁰¹, C₃₋₈cycloalkyl, isoindolino, phthalimidyl, 2-oxo-1,3-oxazolidinyl, aryl or a 5 or 6 membered saturated or unsaturated heterocyclic ring having 1 to 4 heteroatoms selected from the group consisting O, S and N, and optionally substituted by hydroxy, C₁₋₆ alkyl, C₁₋₆ alkoxy, C₁₋₆ alkoxycarbonyl, hydroxyC₁₋₆ alkoxy, oxo, amino, aminoC₁₋₆alkyl, N-(C₁₋₆alkyl)amino, N,N-di(C₁₋₆alkyl)amino, N-(C₁₋₆ acyl)amino, or benzyl,
         wherein
         R²⁰¹ represents hydroxy, amino, N-(C₁₋₆alkyl)amino, N,N-di(C₁₋₆alkyl)amino, N-(halophenylC₁₋₆ alkyl) amino, C₁₋₆alkyl, aminoC₁₋₆ alkyl, aminoC₂₋₆ alkylenyl, C₁₋₆ alkoxy, a 5 or 6 membered saturated or unsaturated heterocyclic ring having 1 to 4 heteroatoms selected from the group consisting O, S and N, and optionally substituted by hydroxy, C₁₋₆ alkyl, C₁₋₆ alkoxy, C₁₋₆ alkoxycarbonyl, hydroxyC₁₋₆ alkoxy, oxo, amino, N-(C₁₋₆alkyl)amino, N,N-di(C₁₋₆alkyl)amino, N-(C₁₋₆ acyl)amino or benzyl;
R³ represents hydrogen, halogen, aminocarbonyl, or C₁₋₆ alkyl optionally substituted by aryl C₁₋₆ alkoxy or mono-, di- or tri-halogen;
R⁴ represents hydrogen or C₁₋₆ alkyl;
R⁵ represents hydrogen or C₁₋₆ alkyl; and
R⁶ represents halogen, hydrogen or C₁₋₆ alkyl,

or a physiologically acceptable salt, solvate, hydrate or stereoisomer thereof,

as a sole active agent,

or of combinations of:

a) such a 2,3-dihydroimidazo[1,2-c]quinazoline compound, or a physiologically acceptable salt, solvate, hydrate or stereoisomer thereof; and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent;

or of pharmaceutical compositions containing such compounds or a physiologically acceptable salt, solvate, hydrate or stereoisomer thereof,

or of pharmaceutical compositions containing such combinations,

for the preparation of a medicament for the treatment or prophylaxis of endometrial cancer (hereinafter abbreviated to “EC”), particularly 1st line, 2nd line, relapsed, refractory, type I or type II EC, or endometriosis.

In a particular embodiment of the above-mentioned first aspect, the present invention relates to the use of a compound selected from the following list,

or a physiologically acceptable salt, solvate, hydrate or stereoisomer thereof,

as a sole active agent,

or of combinations of:

a) such a 2,3-dihydroimidazo[1,2-c]quinazoline compound, or a physiologically acceptable salt, solvate, hydrate or stereoisomer thereof; and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent;

or of pharmaceutical compositions containing such compounds or a physiologically acceptable salt, solvate, hydrate or stereoisomer thereof,

or of pharmaceutical compositions containing such combinations

for the preparation of a medicament for the treatment or prophylaxis of endometrial cancer (hereinafter abbreviated to “EC”), particularly 1st line, 2nd line, relapsed, refractory, type I or type II EC, or endometriosis:

N-(7,8-dimethoxy-2,3-dihydroimidazo[1,2-c]quinazolin-5-yl)nicotinamide;

2-(7,8-dimethoxy-2,3-dihydroimidazo[1,2-c]quinazolin-5-yl)-1-pyridin-3-ylethylenol;

N-(7,8-dimethoxy-2,3-dihydroimidazo[1,2-c]quinazolin-5-yl)-1H-benzimidazole-5-carboxamide;

6-(acetamido)-N-(7,8-dimethoxy-2,3-dihydroimidazo[1,2-c]quinazolin-5-yl)nicotinamide;

N-{5-[2-(7,8-dimethoxy-2,3-dihydroimidazo[1,2-c]quinazolin-5-yl)-1-hydroxyvinyl]pyridin-2-yl}acetamide;

2-({5-[2-hydroxy-2-pyridin-3-ylvinyl]-7-methoxy-2,3-dihydroimidazo[1,2-c]quinazolin-8-yl}oxy)-N,N-dimethylacetamide;

2-[7-methoxy-8-(tetrahydro-2H-pyran-2-ylmethoxy)-2,3-dihydroimidazo[1,2-c]quinazolin-5-yl]-1-pyridin-3-ylethylenol;

2-[8-(2-hydroxyethoxy)-7-methoxy-2,3-dihydroimidazo[1,2-c]quinazolin-5-yl]-1-pyridin-3-ylethylenol;

({5-[2-hydroxy-2-pyridin-3-ylvinyl]-7-methoxy-2,3-dihydroimidazo[1,2-c]quinazolin-8-yl}oxy)acetic acid;

4-({5-[2-hydroxy-2-pyridin-3-ylvinyl]-7-methoxy-2,3-dihydroimidazo[1,2-c]quinazolin-8-yl}oxy)butanoic acid;

({5-[2-hydroxy-2-pyridin-3-ylvinyl]-7-methoxy-2,3-dihydroimidazo[1,2-c]quinazolin-8-yl}oxy)acetonitrile;

2-[7-methoxy-8-(2H-tetrazol-5-ylmethoxy)-2,3-dihydroimidazo[1,2-c]quinazolin-5-yl]-1-pyridin-3-ylethylenol;

2-[7-methoxy-8-(4-morpholin-4-yl-4-oxobutoxy)-2,3-dihydroimidazo[1,2-c]quinazolin-5-yl]-1-pyridin-3-ylethylenol;

5-[1-hydroxy-2-(8-morpholin-4-yl-2,3-dihydroimidazo[1,2-c]quinazolin-5-yl)vinyl]pyridin-3-ol;

N-(2,3-dihydroimidazo[1,2-c]quinazolin-5-yl)-5-hydroxynicotinamide;

6-(acetamido)-N-(7,9-dimethoxy-8-methyl-2,3-dihydroimidazo[1,2-c]quinazolin-5-yl)nicotinamide;

N-(8,9-dimethoxy-2,3-dihydroimidazo[1,2-c]quinazolin-5-yl)-5-hydroxynicotinamide;

5-hydroxy-N-(7-methoxy-2,3-dihydroimidazo[1,2-c]quinazolin-5-yl)nicotinamide;

N-(7,8-dimethoxy-2,3-dihydroimidazo[1,2-c]quinazolin-5-yl)-5-[(4-methoxybenzyl)oxy]nicotinamide;

N-(7,8-dimethoxy-2,3-dihydroimidazo[1,2-c]quinazolin-5-yl)-5-hydroxynicotinamide;

5-hydroxy-N-[8-(trifluoromethyl)-2,3-dihydroimidazo[1,2-c]quinazolin-5-yl]nicotinamide;

N-{8-[3-(1,3-dioxo-1,3-dihydro-2H-isoindol-2-yl)propoxy]-2,3-dihydroimidazo[1,2-c]quinazolin-5-yl}nicotinamide;

N-(7-bromo-8-methoxy-2,3-dihydroimidazo[1,2-c]quinazolin-5-yl)nicotinamide;

6-amino-N-(8-methoxy-2,3-dihydroimidazo[1,2-c]quinazolin-5-yl)nicotinamide;

1-(1H-benzimidazol-5-yl)-2-(8,9-dimethoxy-2,3-dihydroimidazo[1,2-c]quinazolin-5-yl)ethylenol;

2-(8,9-dimethoxy-2,3-dihydroimidazo[1,2-c]quinazolin-5-yl)-1-(2,4-dimethyl-1,3-thiazol-5-yl)ethylenol;

N-(9-methoxy-2,3-dihydroimidazo[1,2-c]quinazolin-5-yl)-1H-benzimidazole-5-carboxamide;

N-(8-bromo-2,3-dihydroimidazo[1,2-c]quinazolin-5-yl)nicotinamide;

N-(8-bromo-2,3-dihydroimidazo[1,2-c]quinazolin-5-yl)-1H-benzimidazole-5-carboxamide;

N-(8-methoxy-2,3-dihydroimidazo[1,2-c]quinazolin-5-yl)-1H-benzimidazole-5-carboxamide;

N-(8-methyl-2,3-dihydroimidazo[1,2-c]quinazolin-5-yl)-1H-benzimidazole-5-carboxamide;

N-[8-(trifluoromethyl)-2,3-dihydroimidazo[1,2-c]quinazolin-5-yl]-1H-benzimidazole-5-carboxamide;

N-(7-fluoro-2,3-dihydroimidazo[1,2-c]quinazolin-5-yl)-1H-benzimidazole-5-carboxamide;

N-(7-methoxy-2,3-dihydroimidazo[1,2-c]quinazolin-5-yl)nicotinamide;

N-(8-chloro-2,3-dihydroimidazo[1,2-c]quinazolin-5-yl)-1H-benzimidazole-5-carboxamide;

6-(acetamido)-N-(8-morpholin-4-yl-2,3-dihydroimidazo[1,2-c]quinazolin-5-yl)nicotinamide;

1-(1H-benzimidazol-5-yl)-2-(8-morpholin-4-yl-2,3-dihydroimidazo[1,2-c]quinazolin-5-yl)ethylenol;

N-{5-[1-hydroxy-2-(8-morpholin-4-yl-2,3-dihydroimidazo[1,2-c]quinazolin-5-yl)vinyl]pyridin-2-yl}acetamide;

6-methyl-N-(8-morpholin-4-yl-2,3-dihydroimidazo[1,2-c]quinazolin-5-yl)nicotinamide;

1-(1H-benzimidazol-5-yl)-2-[8-(4-methylpiperazin-1-yl)-2,3-dihydroimidazo[1,2-c]quinazolin-5-yl]ethylenol;

N-(2,3-dihydroimidazo[1,2-c]quinazolin-5-yl)-3H-imidazo[4,5-b]pyridine-6-carboxamide;

N-(7,8-dimethoxy-2,3-dihydroimidazo[1,2-c]quinazolin-5-yl)-3H-imidazo[4,5-b]pyridine-6-carboxamide;

N-[7-(trifluoromethyl)-2,3-dihydroimidazo[1,2-c]quinazolin-5-yl]-1H-benzimidazole-5-carboxamide;

N-(7,9-dimethoxy-2,3-dihydroimidazo[1,2-c]quinazolin-5-yl)-1H-benzimidazole-5-carboxamide;

N-{5-[2-(7,9-dimethoxy-8-methyl-2,3-dihydroimidazo[1,2-c]quinazolin-5-yl)-1-hydroxyvinyl]pyridin-2-yl}acetamide;

N-{5-[2-(7-bromo-9-methyl-2,3-dihydroimidazo[1,2-c]quinazolin-5-yl)-1-hydroxyvinyl]pyridin-2-yl}acetamide; and

2-(8,9-dimethoxy-2,3-dihydroimidazo[1,2-c]quinazolin-5-yl)-1-pyridin-3-ylethylenol;

Another embodiment of the present invention encompasses the use of a compound having the formula (I):

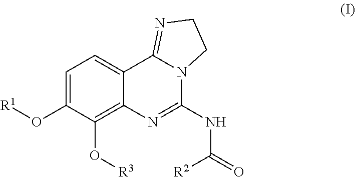

or a physiologically acceptable salt, solvate, hydrate or stereoisomer thereof, in which:

R¹ represents —(CH₂)ₙ—(CHR⁴)—(CH₂)ₘ—N(R⁵)(R^{5′});

R² represents a heteroaryl optionally substituted with 1, 2 or 3 R⁶ groups;

R³ represents alkyl or cycloalkyl;

R⁴ represents hydrogen, hydroxy or alkoxy; and

R⁵ and R^{5′} may be the same or different and represent independently, hydrogen, alkyl, cycloalkylalklyl, or alkoxyalkyl or R⁵ and R^{5′} may be taken together with the nitrogen atom to which they are bound to form a 3-7 membered nitrogen containing heterocyclic ring optionally containing at least one additional heteroatom selected from oxygen, nitrogen or sulfur and which may be optionally substituted with 1 or more R^{6′} groups, or R⁴ and R⁵ may be taken together with the atoms to which they are bound to form a 5-6 membered nitrogen containing heterocyclic ring optionally containing 1 or more nitrogen, oxygen or sulfur atoms and which may be optionally substituted with 1 or more R^{6′} groups; each occurrence of R⁶ may be the same or different and is independently halogen, alkyl, alkenyl, alkynyl, cycloalkyl, cycloalkylalklyl, aryl, arylalkyl, heteroaryl, heteroarylalkyl, heterocyclic ring, heterocyclylalkyl, alkyl-OR⁷, alkyl-SR⁷, alkyl-N(R⁷)(R^{7′}), alkyl-COR⁷, —CN, —COOR⁷, —CON(R⁷)(R^{7′}), —OR⁷, —SR⁷, —N(R⁷)(R^{7′}), or —NR⁷COR⁷ each of which may be optionally substituted with 1 or more R⁸ groups;

each occurrence of R^{6′} may be the same or different and is independently alkyl, cycloalkylalklyl, or alkyl-OR⁷;

each occurrence of R⁷ and R^{7′} may be the same or different and is independently hydrogen, alkyl, alkenyl, alkynyl, cycloalkyl, cycloalkylalklyl, cycloalkenyl, aryl, arylalkyl, heteroaryl, heterocyclic ring, heterocyclylalkyl, or heteroarylalkyl;

each occurrence of R⁸ is independently nitro, hydroxy, cyano, formyl, acetyl, halogen, amino, alkyl, alkoxy, alkenyl, alkynyl, cycloalkyl, cycloalkylalklyl, cycloalkenyl, aryl, arylalkyl, heteroaryl, heterocyclic ring, heterocyclylalkyl, or heteroarylalkyl;

n is an integer from 1-4 and m is an integer from 0-4 with the proviso that when when R⁴ and R⁵ are taken together with the atoms to which they are bound to form a 5-6 membered nitrogen containing ring, n+m≦4;

or a physiologically acceptable salt, solvate, hydrate or stereoisomer thereof, as a sole active agent,

or of combinations of:

a) such a 2,3-dihydroimidazo[1,2-c]quinazoline compound, or a physiologically acceptable salt, solvate, hydrate or stereoisomer thereof; and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent;

or of pharmaceutical compositions containing such compounds or a physiologically acceptable salt, solvate, hydrate or stereoisomer thereof,

or of pharmaceutical compositions containing such combinations,

for the preparation of a medicament for the treatment or prophylaxis of endometrial cancer (hereinafter abbreviated to “EC”), particularly 1st line, 2nd line, relapsed, refractory, type I or type II EC, or endometriosis.

In a preferred embodiment, the invention encompasses the use of a compound of Formula (I), wherein R² is a nitrogen containing heteroaryl optionally substituted with 1, 2 or 3 R⁶ groups,

or a physiologically acceptable salt, solvate, hydrate or stereoisomer thereof,

as a sole active agent,

or of combinations of:

a) such a 2,3-dihydroimidazo[1,2-c]quinazoline compound, or a physiologically acceptable salt, solvate, hydrate or stereoisomer thereof; and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent;

or of pharmaceutical compositions containing such compounds or a physiologically acceptable salt, solvate, hydrate or stereoisomer thereof,

or of pharmaceutical compositions containing such combinations,

for the preparation of a medicament for the treatment or prophylaxis of endometrial cancer (hereinafter abbreviated to “EC”), particularly 1st line, 2nd line, relapsed, refractory, type I or type II EC, or endometriosis.

In another preferred embodiment, the invention encompasses the use of a compound of Formula (I), wherein R⁵ and R^{5′} are independently alkyl,

or a physiologically acceptable salt, solvate, hydrate or stereoisomer thereof,

as a sole active agent,

or of combinations of:

a) such a 2,3-dihydroimidazo[1,2-c]quinazoline compound, or a physiologically acceptable salt, solvate, hydrate or stereoisomer thereof; and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent;

or of pharmaceutical compositions containing such compounds or a physiologically acceptable salt, solvate, hydrate or stereoisomer thereof,

or of pharmaceutical compositions containing such combinations,

for the preparation of a medicament for the treatment or prophylaxis of endometrial cancer (hereinafter abbreviated to “EC”), particularly 1st line, 2nd line, relapsed, refractory, type I or type II EC, or endometriosis.

In still another preferred embodiment, the invention encompasses the use of a compound of Formula (I), wherein R⁵ and R^{5′} are taken together with the nitrogen atom to which they are bound to form a 5-6 membered nitrogen containing heterocyclic ring containing at least one additional heteroatom selected from oxygen, nitrogen or sulfur and which may be optionally substituted with 1 or more R^{6′} groups,

or a physiologically acceptable salt, solvate, hydrate or stereoisomer thereof,

as a sole active agent,

or of combinations of:

a) such a 2,3-dihydroimidazo[1,2-c]quinazoline compound, or a physiologically acceptable salt, solvate, hydrate or stereoisomer thereof; and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent;

or of pharmaceutical compositions containing such compounds or a physiologically acceptable salt, solvate, hydrate or stereoisomer thereof,

or of pharmaceutical compositions containing such combinations,

for the preparation of a medicament for the treatment or prophylaxis of endometrial cancer (hereinafter abbreviated to “EC”), particularly 1st line, 2nd line, relapsed, refractory, type I or type II EC, or endometriosis.

In yet another preferred embodiment, the invention encompasses the use of a compound of Formula (I), wherein R⁴ is hydroxyl,

or a physiologically acceptable salt, solvate, hydrate or stereoisomer thereof,

as a sole active agent,

or of combinations of:

a) such a 2,3-dihydroimidazo[1,2-c]quinazoline compound, or a physiologically acceptable salt, solvate, hydrate or stereoisomer thereof; and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent;

or of pharmaceutical compositions containing such compounds or a physiologically acceptable salt, solvate, hydrate or stereoisomer thereof,

or of pharmaceutical compositions containing such combinations,

for the preparation of a medicament for the treatment or prophylaxis of endometrial cancer (hereinafter abbreviated to “EC”), particularly 1st line, 2nd line, relapsed, refractory, type I or type II EC, or endometriosis.

In another preferred embodiment, the invention encompasses the use of a compound of Formula (I), wherein R⁴ and R⁵ are taken together with the atoms to which they are bound to form a 5-6 membered nitrogen containing heterocyclic ring optionally containing 1 or more nitrogen, oxygen or sulfur atoms and which may be optionally substituted with 1 or more R⁶ groups,

or a physiologically acceptable salt, solvate, hydrate or stereoisomer thereof,

as a sole active agent,

or of combinations of:

a) such a 2,3-dihydroimidazo[1,2-c]quinazoline compound, or a physiologically acceptable salt, solvate, hydrate or stereoisomer thereof; and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent;

or of pharmaceutical compositions containing such compounds or a physiologically acceptable salt, solvate, hydrate or stereoisomer thereof,

or of pharmaceutical compositions containing such combinations,

for the preparation of a medicament for the treatment or prophylaxis of endometrial cancer (hereinafter abbreviated to “EC”), particularly 1st line, 2nd line, relapsed, refractory, type I or type II EC, or endometriosis.

In yet another preferred embodiment, the invention encompasses the use of a compound of Formula (I), wherein R³ is methyl,

or a physiologically acceptable salt, solvate, hydrate or stereoisomer thereof,

as a sole active agent,

or of combinations of:

a) such a 2,3-dihydroimidazo[1,2-c]quinazoline compound, or a physiologically acceptable salt, solvate, hydrate or stereoisomer thereof; and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent;

or of pharmaceutical compositions containing such compounds or a physiologically acceptable salt, solvate, hydrate or stereoisomer thereof,

or of pharmaceutical compositions containing such combinations,

for the preparation of a medicament for the treatment or prophylaxis of endometrial cancer (hereinafter abbreviated to “EC”), particularly 1st line, 2nd line, relapsed, refractory, type I or type II EC, or endometriosis.

In still another preferred embodiment, the invention encompasses the use of a compound of Formula (I), wherein R² is pyridine, pyridazine, pyrimidine, pyrazine, pyrole, oxazole, thiazole, furan or thiophene, optionally substituted with 1, 2 or 3 R⁶ groups; more preferably pyridine, pyridazine, pyrimidine, pyrazine, pyrole, oxazole or thiazole, optionally substituted with 1, 2 or 3 R⁶ groups,

or a physiologically acceptable salt, solvate, hydrate or stereoisomer thereof,

as a sole active agent,

or of combinations of:

a) such a 2,3-dihydroimidazo[1,2-c]quinazoline compound, or a physiologically acceptable salt, solvate, hydrate or stereoisomer thereof; and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent;

or of pharmaceutical compositions containing such compounds or a physiologically acceptable salt, solvate, hydrate or stereoisomer thereof,

or of pharmaceutical compositions containing such combinations,

for the preparation of a medicament for the treatment or prophylaxis of endometrial cancer (hereinafter abbreviated to “EC”), particularly 1st line, 2nd line, relapsed, refractory, type I or type II EC, or endometriosis.

In a distinct embodiment, the invention encompasses the use of a compound of formula (Ia)

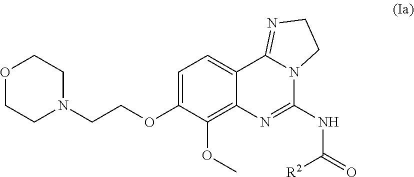

or a physiologically acceptable salt, solvate, hydrate or stereoisomer thereof,

wherein R² is as defined above,

or a physiologically acceptable salt, solvate, hydrate or stereoisomer thereof,

as a sole active agent,

or of combinations of:

a) such a 2,3-dihydroimidazo[1,2-c]quinazoline compound, or a physiologically acceptable salt, solvate, hydrate or stereoisomer thereof; and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent;

or of pharmaceutical compositions containing such compounds or a physiologically acceptable salt, solvate, hydrate or stereoisomer thereof,

or of pharmaceutical compositions containing such combinations, for the preparation of a medicament for the treatment or prophylaxis of endometrial cancer (hereinafter abbreviated to “EC”), particularly 1st line, 2nd line, relapsed, refractory, type I or type II EC, or endometriosis.

In another distinct embodiment, the invention encompasses the use of a compound of formula (Ib):

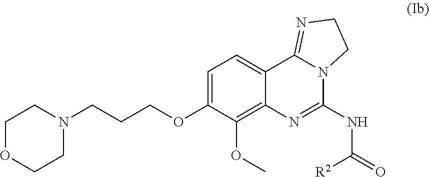

or a physiologically acceptable salt, solvate, hydrate or stereoisomer thereof,

wherein R² is as defined above,

or a physiologically acceptable salt, solvate, hydrate or stereoisomer thereof,

as a sole active agent,

or of combinations of:

a) such a 2,3-dihydroimidazo[1,2-c]quinazoline compound, or a physiologically acceptable salt, solvate, hydrate or stereoisomer thereof; and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent;

or of pharmaceutical compositions containing such compounds or a physiologically acceptable salt, solvate, hydrate or stereoisomer thereof,

or of pharmaceutical compositions containing such combinations,

for the preparation of a medicament for the treatment or prophylaxis of endometrial cancer (hereinafter abbreviated to “EC”), particularly 1st line, 2nd line, relapsed, refractory, type I or type II EC, or endometriosis.

In still another distinct embodiment, the invention encompasses the use of a compound of formula (Ic):

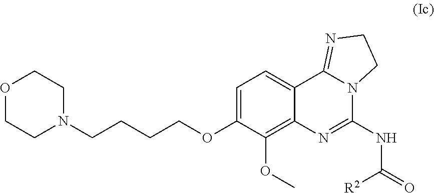

or a physiologically acceptable salt, solvate, hydrate or stereoisomer thereof,

wherein R² is as defined above,

or a physiologically acceptable salt, solvate, hydrate or stereoisomer thereof,

as a sole active agent,

or of combinations of:

a) such a 2,3-dihydroimidazo[1,2-c]quinazoline compound, or a physiologically acceptable salt, solvate, hydrate or stereoisomer thereof; and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent;

or of pharmaceutical compositions containing such compounds or a physiologically acceptable salt, solvate, hydrate or stereoisomer thereof,

or of pharmaceutical compositions containing such combinations,

for the preparation of a medicament for the treatment or prophylaxis of endometrial cancer (hereinafter abbreviated to “EC”), particularly 1st line, 2nd line, relapsed, refractory, type I or type II EC, or endometriosis.

In yet another distinct embodiment, the invention encompasses the use of a compound of the formula (Id):

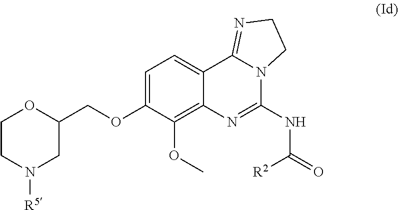

or a physiologically acceptable salt, solvate, hydrate or stereoisomer thereof,

wherein R² and R⁴ are as defined above,

or a physiologically acceptable salt, solvate, hydrate or stereoisomer thereof,

as a sole active agent,

or of combinations of:

a) such a 2,3-dihydroimidazo[1,2-c]quinazoline compound, or a physiologically acceptable salt, solvate, hydrate or stereoisomer thereof; and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent;

or of pharmaceutical compositions containing such compounds or a physiologically acceptable salt, solvate, hydrate or stereoisomer thereof,

or of pharmaceutical compositions containing such combinations,

for the preparation of a medicament for the treatment or prophylaxis of endometrial cancer (hereinafter abbreviated to “EC”), particularly 1st line, 2nd line, relapsed, refractory, type I or type II EC, or endometriosis.

In yet another distinct embodiment, the invention encompasses the use of a compound of the formula (Ie):

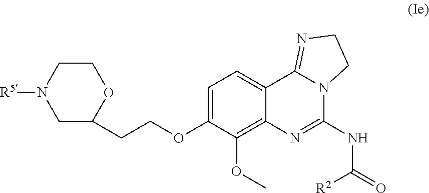

or a physiologically acceptable salt, solvate, hydrate or stereoisomer thereof,

wherein R² and R⁴ are as defined above,

or a physiologically acceptable salt, solvate, hydrate or stereoisomer thereof,

as a sole active agent,

or of combinations of:

a) such a 2,3-dihydroimidazo[1,2-c]quinazoline compound, or a physiologically acceptable salt, solvate, hydrate or stereoisomer thereof; and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent;

or of pharmaceutical compositions containing such compounds or a physiologically acceptable salt, solvate, hydrate or stereoisomer thereof,

or of pharmaceutical compositions containing such combinations,

for the preparation of a medicament for the treatment or prophylaxis of endometrial cancer (hereinafter abbreviated to “EC”), particularly 1st line, 2nd line, relapsed, refractory, type I or type II EC, or endometriosis.

In a preferred embodiment, the invention encompasses the use of a compound of formula (I)-(V), wherein R² is pyridine, pyridazine, pyrimidine, pyrazine, pyrole, oxazole, thiazole, furan or thiophene, optionally substituted with 1, 2 or 3 R⁶ groups; more preferably wherein R² is pyridine, pyridazine, pyrimidine, pyrazine, pyrole, oxazole or thiazole, optionally substituted with 1, 2 or 3 R⁶ groups,

or a physiologically acceptable salt, solvate, hydrate or stereoisomer thereof,

as a sole active agent,

or of combinations of:

a) such a 2,3-dihydroimidazo[1,2-c]quinazoline compound, or a physiologically acceptable salt, solvate, hydrate or stereoisomer thereof; and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent;

or of pharmaceutical compositions containing such compounds or a physiologically acceptable salt, solvate, hydrate or stereoisomer thereof,

or of pharmaceutical compositions containing such combinations, for the preparation of a medicament for the treatment or prophylaxis of endometrial cancer (hereinafter abbreviated to “EC”), particularly 1st line, 2nd line, relapsed, refractory, type I or type II EC, or endometriosis.

In still another preferred embodiment, the invention encompasses the use of a compound having the formula:
N-[7-methoxy-8-(3-morpholin-4-ylpropoxy)-2,3-dihydroimidazo[1,2-c]quinazolin-5-yl]pyrimidine-5-carboxamide;
N-(8-{3-[(2R,6S)-2,6-dimethylmorpholin-4-yl]propoxy}-7-methoxy-2,3-dihydroimidazo[1,2-c]quinazolin-5-yl)nicotinamide;
N-(8-{3-[(2R,6S)-2,6-dimethylmorpholin-4-yl]propoxy}-7-methoxy-2,3-dihydroimidazo[1,2-c]quinazolin-5-yl)-2,4-dimethyl-1,3-thiazole-5-carboxamide;
2-amino-N-[7-methoxy-8-(3-morpholin-4-ylpropoxy)-2,3-dihydroimidazo[1,2-c]quinazolin-5-yl]-1,3-thiazole-5-carboxamide;
2-amino-N-[7-methoxy-8-(3-morpholin-4-ylpropoxy)-2,3-dihydroimidazo[1,2-c]quinazolin-5-yl]isonicotinamide;
2-amino-N-[7-methoxy-8-(3-morpholin-4-ylpropoxy)-2,3-dihydroimidazo[1,2-c]quinazolin-5-yl]-4-methyl-1,3-thiazole-5-carboxamide;
2-amino-N-[7-methoxy-8-(3-morpholin-4-ylpropoxy)-2,3-dihydroimidazo[1,2-c]quinazolin-5-yl]-4-propylpyrimidine-5-carboxamide;
N-{8-[2-(4-ethylmorpholin-2-yl)ethoxy]-7-methoxy-2,3-dihydroimidazo[1,2-c]quinazolin-5-yl}nicotinamide;
N-{8-[2-(dimethylamino)ethoxy]-7-methoxy-2,3-dihydroimidazo[1,2-c]quinazolin-5-yl}pyrimidine-5-carboxamide;
N-(8-{3-[2-(hydroxymethyl)morpholin-4-yl]propoxy}-7-methoxy-2,3-dihydroimidazo[1,2-c]quinazolin-5-yl)nicotinamide;
N-(8-{3-[2-(hydroxymethyl)morpholin-4-yl]propoxy}-7-methoxy-2,3-dihydroimidazo[1,2-c]quinazolin-5-yl)nicotinamide;
N-{8-[3-(dimethylamino)propoxy]-7-methoxy-2,3-dihydroimidazo[1,2-c]quinazolin-5-yl}nicotinamide 1-oxide;
2-amino-N-[7-methoxy-8-(3-morpholin-4-ylpropoxy)-2,3-dihydroimidazo[1,2-c]quinazolin-5-yl]pyrimidine-5-carboxamide;
N-[7-methoxy-8-(3-morpholin-4-ylpropoxy)-2,3-dihydroimidazo[1,2-c]quinazolin-5-yl]-6-(2-pyrrolidin-1-ylethyl)nicotinamide;
6-(cyclopentylamino)-N-[7-methoxy-8-(3-morpholin-4-ylpropoxy)-2,3-dihydroimidazo[1,2-c]quinazolin-5-yl]nicotinamide;
N-[8-(2-hydroxy-3-morpholin-4-ylpropoxy)-7-methoxy-2,3-dihydroimidazo[1,2-c]quinazolin-5-yl]nicotinamide;
N-{7-methoxy-8-[3-(3-methylmorpholin-4-yl)propoxy]-2,3-dihydroimidazo[1,2-c]quinazolin-5-yl}nicotinamide;
N-(8-{3-[2-(hydroxymethyl)morpholin-4-yl]propoxy}-7-methoxy-2,3-dihydroimidazo[1,2-c]quinazolin-5-yl)nicotinamide;
N-(8-{2-[4-(cyclobutylmethyl)morpholin-2-yl]ethoxy}-7-methoxy-2,3-dihydroimidazo[1,2-c]quinazolin-5-yl)nicotinamide;
N-(7-methoxy-8-{2-[4-(2-methoxyethyl)morpholin-2-yl]ethoxy}-2,3-dihydroimidazo[1,2-c]quinazolin-5-yl)nicotinamide;
N-{8-[(4-ethylmorpholin-2-yl)methoxy]-7-methoxy-2,3-dihydroimidazo[1,2-c]quinazolin-5-yl}nicotinamide;
N-(7-methoxy-8-{[4-(2-methoxyethyl)morpholin-2-yl]methoxy}-2,3-dihydroimidazo[1,2-c]quinazolin-5-yl)nicotinamide;
N-{7-methoxy-8-[(4-methylmorpholin-2-yl)methoxy]-2,3-dihydroimidazo[1,2-c]quinazolin-5-yl}nicotinamide;
N-[7-methoxy-8-(3-morpholin-4-ylpropoxy)-2,3-dihydroimidazo[1,2-c]quinazolin-5-yl]pyrimidine-4-carboxamide;
2-amino-N-[7-methoxy-8-(3-morpholin-4-ylpropoxy)-2,3-dihydroimidazo[1,2-c]quinazolin-5-yl]pyrimidine-4-carboxamide;
N-[7-methoxy-8-(3-morpholin-4-ylpropoxy)-2,3-dihydroimidazo[1,2-c]quinazolin-5-yl]-1-methyl-1H-imidazole-4-carboxamide;
rel-N-(8-{3-[(2R,6S)-2,6-dimethylmorpholin-4-yl]propoxy}-7-methoxy-2,3-dihydroimidazo[1,2-c]quinazolin-5-yl)pyrimidine-5-carboxamide;
rel-N-(8-{3-[(2R,6S)-2,6-dimethylmorpholin-4-yl]propoxy}-7-methoxy-2,3-dihydroimidazo[1,2-c]quinazolin-5-yl)-6-methylnicotinamide;
rel-6-acetamido-N-(8-{3-[(2R,6S)-2,6-dimethylmorpholin-4-yl]propoxy}-7-methoxy-2,3-dihydroimidazo[1,2-c]quinazolin-5-yl)nicotinamide;
N-[7-methoxy-8-(3-morpholin-4-ylpropoxy)-2,3-dihydroimidazo[1,2-c]quinazolin-5-yl]-1-methyl-1H-imidazole-5-carboxamide;
6-amino-N-[7-methoxy-8-(3-morpholin-4-ylpropoxy)-2,3-dihydroimidazo[1,2-c]quinazolin-5-yl]-2-methylnicotinamide;
2-amino-N-[7-methoxy-8-(3-morpholin-4-ylpropoxy)-2,3-dihydroimidazo[1,2-c]quinazolin-5-yl]-4-methylpyrimidine-5-carboxamide;
6-amino-5-bromo-N-[7-methoxy-8-(3-morpholin-4-ylpropoxy)-2,3-dihydroimidazo[1,2-c]quinazolin-5-yl]nicotinamide;
2-amino-N-[7-methoxy-8-(3-morpholin-4-ylpropoxy)-2,3-dihydroimidazo[1,2-c]quinazolin-5-yl]-1,3-oxazole-5-carboxamide;
N-[7-methoxy-8-(morpholin-2-ylmethoxy)-2,3-dihydroimidazo[1,2-c]quinazolin-5-yl]nicotinamide;
2-{[2-(dimethylamino)ethyl]amino}-N-{8-[3-(dimethylamino)propoxy]-7-methoxy-2,3-dihydroimidazo[1,2-c]quinazolin-5-yl}pyrimidine-5-carboxamide;
2-amino-N-{8-[3-(dimethylamino)propoxy]-7-methoxy-2,3-dihydroimidazo[1,2-c]quinazolin-5-yl}-1,3-thiazole-5-carboxamide;
rel-2-amino-N-(8-{3-[(2R,6S)-2,6-dimethylmorpholin-4-yl]propoxy}-7-methoxy-2,3-dihydroimidazo[1,2-c]quinazolin-5-yl)pyrimidine-5-carboxamide;
rel-6-amino-N-(8-{3-[(2R,6S)-2,6-dimethylmorpholin-4-yl]propoxy}-7-methoxy-2,3-dihydroimidazo[1,2-c]quinazolin-5-yl)nicotinamide;
2-[(2-hydroxyethyl)amino]-N-[7-methoxy-8-(3-morpholin-4-ylpropoxy)-2,3-dihydroimidazo[1,2-c]quinazolin-5-yl]pyrimidine-5-carboxamide;
N-[7-methoxy-8-(3-morpholin-4-ylpropoxy)-2,3-dihydroimidazo[1,2-c]quinazolin-5-yl]-2-[(3-methoxypropyl)amino]pyrimidine-5-carboxamide;
2-amino-N-{8-[3-(dimethylamino)propoxy]-7-methoxy-2,3-dihydroimidazo[1,2-c]quinazolin-5-yl}pyrimidine-5-carboxamide;
N-[7-methoxy-8-(3-morpholin-4-ylpropoxy)-2,3-dihydroimidazo[1,2-c]quinazolin-5-yl]-2-[(3-morpholin-4-ylpropyl)amino]pyrimidine-5-carboxamide;
2-[(2-methoxyethyl)amino]-N-[7-methoxy-8-(3-morpholin-4-ylpropoxy)-2,3-dihydroimidazo[1,2-c]quinazolin-5-yl]pyrimidine-5-carboxamide;
2-{[2-(dimethylamino)ethyl]amino}-N-[7-methoxy-8-(3-morpholin-4-ylpropoxy)-2,3-dihydroimidazo[1,2-c]quinazolin-5-yl]pyrimidine-5-carboxamide;
6-amino-N-{8-[3-(dimethylamino)propoxy]-7-methoxy-2,3-dihydroimidazo[1,2-c]quinazolin-5-yl}nicotinamide;
N-[7-methoxy-8-(3-morpholin-4-ylpropoxy)-2,3-dihydroimidazo[1,2-c]quinazolin-5-yl]-2-pyrrolidin-1-ylpyrimidine-5-carboxamide;
N-[7-methoxy-8-(3-morpholin-4-ylpropoxy)-2,3-dihydroimidazo[1,2-c]quinazolin-5-yl]-2-(4-methylpiperazin-1-yl)pyrimidine-5-carboxamide;
N-[7-methoxy-8-(3-morpholin-4-ylpropoxy)-2,3-dihydroimidazo[1,2-c]quinazolin-5-yl]-2-morpholin-4-ylpyrimidine-5-carboxamide;
N-[7-methoxy-8-(3-morpholin-4-ylpropoxy)-2,3-dihydroimidazo[1,2-c]quinazolin-5-yl]-6-piperazin-1-ylnicotinamide hydrochloride;
6-[(3S)-3-aminopyrrolidin-1-yl]-N-[7-methoxy-8-(3-morpholin-4-ylpropoxy)-2,3-dihydroimidazo[1,2-c]quinazolin-5-yl]nicotinamide hydrochloride hydrate;
6-[(3R)-3-aminopyrrolidin-1-yl]-N-[7-methoxy-8-(3-morpholin-4-ylpropoxy)-2,3-dihydroimidazo[1,2-c]quinazolin-5-yl]nicotinamide hydrochloride;
6-[(4-fluorobenzyl)amino]-N-[7-methoxy-8-(3-morpholin-4-ylpropoxy)-2,3-dihydroimidazo[1,2-c]quinazolin-5-yl]nicotinamide;
6-[(2-furylmethyl)amino]-N-[7-methoxy-8-(3-morpholin-4-ylpropoxy)-2,3-dihydroimidazo[1,2-c]quinazolin-5-yl]nicotinamide;
6-[(2-methoxyethyl)amino]-N-[7-methoxy-8-(3-morpholin-4-ylpropoxy)-2,3-dihydroimidazo[1,2-c]quinazolin-5-yl]nicotinamide;
N-[7-methoxy-8-(3-morpholin-4-ylpropoxy)-2,3-dihydroimidazo[1,2-c]quinazolin-5-yl]-6-(1H-pyrrol-1-yl)nicotinamide;
N-[7-methoxy-8-(3-morpholin-4-ylpropoxy)-2,3-dihydroimidazo[1,2-c]quinazolin-5-yl]-6-morpholin-4-ylnicotinamide;
N-{7-methoxy-8-[3-(methylamino)propoxy]-2,3-dihydroimidazo[1,2-c]quinazolin-5-yl}nicotinamide;
6-[(2,2-dimethylpropanoyl)amino]-N-[7-methoxy-8-(3-morpholin-4-ylpropoxy)-2,3-dihydroimidazo[1,2-c]quinazolin-5-yl]nicotinamide;
6-[(cyclopropylcarbonyl)amino]-N-[7-methoxy-8-(3-morpholin-4-ylpropoxy)-2,3-dihydroimidazo[1,2-c]quinazolin-5-yl]nicotinamide
N-[7-methoxy-8-(3-morpholin-4-ylpropoxy)-2,3-dihydroimidazo[1,2-c]quinazolin-5-yl]-6-(2,2,2-trifluoroethoxy)nicotinamide;
N-[7-methoxy-8-(3-morpholin-4-ylpropoxy)-2,3-dihydroimidazo[1,2-c]quinazolin-5-yl]-6-(trifluoromethyl)nicotinamide;
6-(isobutyrylamino)-N-[7-methoxy-8-(3-morpholin-4-ylpropoxy)-2,3-dihydroimidazo[1,2-c]quinazolin-5-yl]nicotinamide;
N-{7-methoxy-8-[3-(4-methylpiperazin-1-yl)propoxy]-2,3-dihydroimidazo[1,2-c]quinazolin-5-yl}nicotinamide;
N-[7-methoxy-8-(3-morpholin-4-ylpropoxy)-2,3-dihydroimidazo[1,2-c]quinazolin-5-yl]-2-{[(methylamino)carbonyl]amino}-1,3-thiazole-4-carboxamide;
N-[7-methoxy-8-(3-morpholin-4-ylpropoxy)-2,3-dihydroimidazo[1,2-c]quinazolin-5-yl]-6-{[(methylamino)carbonyl]amino}nicotinamide;
N-[7-methoxy-8-(3-morpholin-4-ylpropoxy)-2,3-dihydroimidazo[1,2-c]quinazolin-5-yl]-2-(methylamino)-1,3-thiazole-4-carboxamide;
N-[7-methoxy-8-(2-morpholin-4-ylethoxy)-2,3-dihydroimidazo[1,2-c]quinazolin-5-yl]nicotinamide;
N-{8-[2-(dimethylamino)ethoxy]-7-methoxy-2,3-dihydroimidazo[1,2-c]quinazolin-5-yl}-2,4-dimethyl-1,3-thiazole-5-carboxamide;
N-{8-[2-(dimethylamino)ethoxy]-7-methoxy-2,3-dihydroimidazo[1,2-c]quinazolin-5-yl}-6-methylnicotinamide;
6-{[(isopropylamino)carbonyl]amino}-N-[7-methoxy-8-(3-morpholin-4-ylpropoxy)-2,3-dihydroimidazo[1,2-c]quinazolin-5-yl]nicotinamide;
N-[7-methoxy-8-(3-morpholin-4-ylpropoxy)-2,3-dihydroimidazo[1,2-c]quinazolin-5-yl]-6-pyrrolidin-1-ylnicotinamide;
6-(dimethylamino)-N-[7-methoxy-8-(3-morpholin-4-ylpropoxy)-2,3-dihydroimidazo[1,2-c]quinazolin-5-yl]nicotinamide;
N-[7-methoxy-8-(3-piperidin-1-ylpropoxy)-2,3-dihydroimidazo[1,2-c]quinazolin-5-yl]nicotinamide;
N-[7-methoxy-8-(2-pyrrolidin-1-ylethoxy)-2,3-dihydroimidazo[1,2-c]quinazolin-5-yl]nicotinamide;
N-[7-methoxy-8-(2-piperidin-1-ylethoxy)-2,3-dihydroimidazo[1,2-c]quinazolin-5-yl]nicotinamide;
6-{[(ethylamino)carbonyl]amino}-N-[7-methoxy-8-(3-morpholin-4-ylpropoxy)-2,3-dihydroimidazo[1,2-c]quinazolin-5-yl]nicotinamide;
6-fluoro-N-[7-methoxy-8-(3-morpholin-4-ylpropoxy)-2,3-dihydroimidazo[1,2-c]quinazolin-5-yl]nicotinamide;
2-amino-N-[7-methoxy-8-(3-morpholin-4-ylpropoxy)-2,3-dihydroimidazo[1,2-c]quinazolin-5-yl]-1,3-oxazole-4-carboxamide;
2-(ethylamino)-N-[7-methoxy-8-(3-morpholin-4-ylpropoxy)-2,3-dihydroimidazo[1,2-c]quinazolin-5-yl]-1,3-thiazole-4-carboxamide;
N-[7-methoxy-8-(3-morpholin-4-ylpropoxy)-2,3-dihydroimidazo[1,2-c]quinazolin-5-yl]pyrazine-2-carboxamide;
N-[8-(2-aminoethoxy)-7-methoxy-2,3-dihydroimidazo[1,2-c]quinazolin-5-yl]nicotinamide;
6-amino-N-[7-methoxy-8-(3-morpholin-4-ylpropoxy)-2,3-dihydroimidazo[1,2-c]quinazolin-5-yl]nicotinamide;
N-[7-methoxy-8-(3-morpholin-4-ylpropoxy)-2,3-dihydroimidazo[1,2-c]quinazolin-5-yl]isonicotinamide;
N-{8-[3-(diethylamino)propoxy]-7-methoxy-2,3-dihydroimidazo[1,2-c]quinazolin-5-yl}nicotinamide;
N-{8-[2-(diisopropylamino)ethoxy]-7-methoxy-2,3-dihydroimidazo[1,2-c]quinazolin-5-yl}nicotinamide;
N-{8-[2-(diethylamino)ethoxy]-7-methoxy-2,3-dihydroimidazo[1,2-c]quinazolin-5-yl}nicotinamide;
N-{8-[3-(dimethylamino)propoxy]-7-methoxy-2,3-dihydroimidazo[1,2-c]quinazolin-5-yl}nicotinamide;
N-{8-[2-(dimethylamino)ethoxy]-7-methoxy-2,3-dihydroimidazo[1,2-c]quinazolin-5-yl}nicotinamide;
N-[7-methoxy-8-(3-morpholin-4-ylpropoxy)-2,3-dihydroimidazo[1,2-c]quinazolin-5-yl]-2-(methylamino)pyrimidine-5-carboxamide;
N-[7-methoxy-8-(3-morpholin-4-ylpropoxy)-2,3-dihydroimidazo[1,2-c]quinazolin-5-yl]-2-(methylthio)pyrimidine-5-carboxamide;
N-[8-(3-aminopropoxy)-7-methoxy-2,3-dihydroimidazo[1,2-c]quinazolin-5-yl]nicotinamide trifluoroacetate;
N-[7-methoxy-8-(3-morpholin-4-ylpropoxy)-2,3-dihydroimidazo[1,2-c]quinazolin-5-yl]thiophene-2-carboxamide;
N-[7-methoxy-8-(3-morpholin-4-ylpropoxy)-2,3-dihydroimidazo[1,2-c]quinazolin-5-yl]-2,4-dimethyl-1,3-thiazole-5-carboxamide;
2-methoxy-N-[7-methoxy-8-(3-morpholin-4-ylpropoxy)-2,3-dihydroimidazo[1,2-c]quinazolin-5-yl]pyrimidine-5-carboxamide;
N-[7-methoxy-8-(3-morpholin-4-ylpropoxy)-2,3-dihydroimidazo[1,2-c]quinazolin-5-yl]-3-furamide;
N-[7-methoxy-8-(3-morpholin-4-ylpropoxy)-2,3-dihydroimidazo[1,2-c]quinazolin-5-yl]thiophene-3-carboxamide;
N-[7-methoxy-8-(3-morpholin-4-ylpropoxy)-2,3-dihydroimidazo[1,2-c]quinazolin-5-yl]-2-methyl-1,3-thiazole-4-carboxamide;
6-methoxy-N-[7-methoxy-8-(3-morpholin-4-ylpropoxy)-2,3-dihydroimidazo[1,2-c]quinazolin-5-yl]nicotinamide;
5-methoxy-N-[7-methoxy-8-(3-morpholin-4-ylpropoxy)-2,3-dihydroimidazo[1,2-c]quinazolin-5-yl]nicotinamide;
N-[7-methoxy-8-(3-morpholin-4-ylpropoxy)-2,3-dihydroimidazo[1,2-c]quinazolin-5-yl]-6-methylnicotinamide;
6-(acetylamino)-N-[7-methoxy-8-(3-morpholin-4-ylpropoxy)-2,3-dihydroimidazo[1,2-c]quinazolin-5-yl]nicotinamide;
N-[7-methoxy-8-(3-morpholin-4-ylpropoxy)-2,3-dihydroimidazo[1,2-c]quinazolin-5-yl]nicotinamide;

or a physiologically acceptable salt, solvate, hydrate or stereoisomer thereof,

as a sole active agent,

or of combinations of:

a) such a 2,3-dihydroimidazo[1,2-c]quinazoline compound, or a physiologically acceptable salt, solvate, hydrate or stereoisomer thereof; and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent;

or of pharmaceutical compositions containing such compounds or a physiologically acceptable salt, solvate, hydrate or stereoisomer thereof,

or of pharmaceutical compositions containing such combinations,

for the preparation of a medicament for the treatment or prophylaxis of endometrial cancer (hereinafter abbreviated to “EC”), particularly 1st line, 2nd line, relapsed, refractory, type I or type II EC, or endometriosis.

In a preferred embodiment, the invention encompasses the use of a compound having the formula:
N-[7-methoxy-8-(3-morpholin-4-ylpropoxy)-2,3-dihydroimidazo[1,2-c]quinazolin-5-yl]nicotinamide;
N-[7-methoxy-8-(3-morpholin-4-ylpropoxy)-2,3-dihydroimidazo[1,2-c]quinazolin-5-yl]-6-methylnicotinamide;
5-methoxy-N-[7-methoxy-8-(3-morpholin-4-ylpropoxy)-2,3-dihydroimidazo[1,2-c]quinazolin-5-yl]nicotinamide;
N-[7-methoxy-8-(3-morpholin-4-ylpropoxy)-2,3-dihydroimidazo[1,2-c]quinazolin-5-yl]-2,4-dimethyl-1,3-thiazole-5-carboxamide;
N-{8-[2-(dimethylamino)ethoxy]-7-methoxy-2,3-dihydroimidazo[1,2-c]quinazolin-5-yl}nicotinamide;
N-{8-[3-(dimethylamino)propoxy]-7-methoxy-2,3-dihydroimidazo[1,2-c]quinazolin-5-yl}nicotinamide;
6-{[(isopropylamino)carbonyl]amino}-N-[7-methoxy-8-(3-morpholin-4-ylpropoxy)-2,3-dihydroimidazo[1,2-c]quinazolin-5-yl]nicotinamide;
N-{8-[2-(dimethylamino)ethoxy]-7-methoxy-2,3-dihydroimidazo[1,2-c]quinazolin-5-yl}-2,4-dimethyl-1,3-thiazole-5-carboxamide;
N-[7-methoxy-8-(2-morpholin-4-ylethoxy)-2,3-dihydroimidazo[1,2-c]quinazolin-5-yl]nicotinamide;
rel-6-amino-N-(8-{3-[(2R,6S)-2,6-dimethylmorpholin-4-yl]propoxy}-7-methoxy-2,3-dihydroimidazo[1,2-c]quinazolin-5-yl)nicotinamide;
rel-2-amino-N-(8-{3-[(2R,6S)-2,6-dimethylmorpholin-4-yl]propoxy}-7-methoxy-2,3-dihydroimidazo[1,2-c]quinazolin-5-yl)pyrimidine-5-carboxamide;
2-amino-N-[7-methoxy-8-(3-morpholin-4-ylpropoxy)-2,3-dihydroimidazo[1,2-c]quinazolin-5-yl]pyrimidine-5-carboxamide;
N-{8-[2-(dimethylamino)ethoxy]-7-methoxy-2,3-dihydroimidazo[1,2-c]quinazolin-5-yl}pyrimidine-5-carboxamide;
N-[7-methoxy-8-(3-morpholin-4-ylpropoxy)-2,3-dihydroimidazo[1,2-c]quinazolin-5-yl]pyrimidine-5-carboxamide;

or a physiologically acceptable salt, solvate, hydrate or stereoisomer thereof,

as a sole active agent,

or of combinations of:

a) such a 2,3-dihydroimidazo[1,2-c]quinazoline compound, or a physiologically acceptable salt, solvate, hydrate or stereoisomer thereof; and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent;

or of pharmaceutical compositions containing such compounds or a physiologically acceptable salt, solvate, hydrate or stereoisomer thereof,

or of pharmaceutical compositions containing such combinations,

for the preparation of a medicament for the treatment or prophylaxis of endometrial cancer (hereinafter abbreviated to “EC”), particularly 1st line, 2nd line, relapsed, refractory, type I or type II EC, or endometriosis.

In a preferred embodiment, the invention encompasses the use of a compound having the formula:

2-amino-N-[7-methoxy-8-(3-morpholin-4-ylpropoxy)-2,3-dihydroimidazo[1,2-c]quinazolin-5-yl]pyrimidine-5-carboxamide, or a physiologically acceptable salt, solvate, hydrate or stereoisomer thereof;

as a sole active agent,

or of pharmaceutical compositions containing such a compound or a physiologically acceptable salt, solvate, hydrate or stereoisomer thereof,

for the preparation of a medicament for the treatment or prophylaxis of endometrial cancer (hereinafter abbreviated to “EC”), particularly 1st line, 2nd line, relapsed, refractory, type I or type II EC, or endometriosis.

In a preferred embodiment, the invention encompasses the use of a compound having the formula:

2-amino-N-[7-methoxy-8-(3-morpholin-4-ylpropoxy)-2,3-dihydroimidazo[1,2-c]quinazolin-5-yl]pyrimidine-5-carboxamide dihydrochloride;

as a sole active agent,

or of pharmaceutical compositions containing such a compound or a physiologically acceptable salt, solvate, hydrate or stereoisomer thereof,

for the preparation of a medicament for the treatment or prophylaxis of endometrial cancer (hereinafter abbreviated to “EC”), particularly 1st line, 2nd line, relapsed, refractory, type I or type II EC, or endometriosis.

In a preferred embodiment, the invention encompasses the use of combinations of:

a) 2-amino-N-[7-methoxy-8-(3-morpholin-4-ylpropoxy)-2,3-dihydroimidazo[1,2-c]quinazolin-5-yl]pyrimidine-5-carboxamide, or a physiologically acceptable salt, solvate, hydrate or stereoisomer thereof; and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent;

or of pharmaceutical compositions containing such a compound or a physiologically acceptable salt, solvate, hydrate or stereoisomer thereof,

or of pharmaceutical compositions containing such combinations, for the preparation of a medicament for the treatment or prophylaxis of endometrial cancer (hereinafter abbreviated to “EC”), particularly 1st line, 2nd line, relapsed, refractory, type I or type II EC, or endometriosis.

Where there is a discrepancy between the chemical name and the chemical structure depicted, the chemical structure depicted takes precedence over the chemical name given.

Without being bound by theory or mechanism, the compounds of the present invention display surprising activity for the inhibition of phosphatidylinositol-3-kinase and chemical and structural stability over those compounds of the prior art. It is believed that this surprising activity is based on the chemical structure of the compounds, in particular the basicity of the compounds as a result of R¹ being amino optionally substituted with R⁵ and R^{5′}. Further, the appropriate choice of R³ and R² provide the necessary activity against the appropriate isoforms to allow for activity in vivo.

In accordance a particular embodiment of any of the above aspects, or embodiments thereof, of the present invention, said cancer is endometrial cancer (hereinafter abbreviated to “EC”), particularly 1st line, 2nd line, relapsed, refractory, type I or type II EC, or endometriosis.

Definitions

The term ‘alkyl’ refers to a straight or branched hydrocarbon chain radical consisting solely of carbon and hydrogen atoms, containing solely of carbon and hydrogen atoms, containing no unsaturation, having from one to eight carbon atoms, and which is attached to the rest of the molecule by a single bond, such as illustratively, methyl, ethyl, n-propyl 1-methylethyl (isopropyl), n-butyl, n-pentyl, and 1,1-dimethylethyl (t-butyl).

The term “alkenyl ” refers to an aliphatic hydrocarbon group containing a carbon-carbon double bond and which may be a straight or branched or branched chain having about 2 to about 10 carbon atoms, e.g., ethenyl, 1-propenyl, 2-propenyl (allyl), iso-propenyl, 2-methyl-1-propenyl, 1-butenyl, 2-and butenyl.

The term “alkynyl” refers to a straight or branched chain hydrocarbonyl radicals having at least one carbon-carbon triple bond, and having in the range of about 2 up to 12 carbon atoms (with radicals having in the range of about 2 up to 10 carbon atoms presently being preferred) e.g., ethynyl.

The term “alkoxy” denotes an alkyl group as defined herein attached via oxygen linkage to the rest of the molecule. Representative examples of those groups are methoxy and ethoxy.

The term “alkoxyakyl” denotes an alkoxy group as defined herein attached via oxygen linkage to an alkyl group which is then attached to the main structure at any carbon from alkyl group that results in the creation of a stable structure the rest of the molecule. Representative examples of those groups are —CH₂OCH₃, —CH₂OC₂H₅.

The term “cycloalkyl” denotes a non-aromatic mono or multicyclic ring system of about 3 to 12 carbon atoms such as cyclopropyl, cyclobutyl, cyclopentyl, cyclohexyl and examples of multicyclic cycloalkyl groups include perhydronapththyl, adamantyl and norbornyl groups bridged cyclic group or sprirobicyclic groups e.g sprio (4,4) non-2-yl.

The term “cycloalkylalkyl” refers to cyclic ring-containing radicals containing in the range of about about 3 up to 8 carbon atoms directly attached to alkyl group which is then also attached to the main structure at any carbon from the alkyl group that results in the creation of a stable structure such as cyclopropylmethyl, cyclobuyylethyl, cyclopentylethyl.

The term “aryl” refers to aromatic radicals having in the range of 6 up to 14 carbon atoms such as phenyl, naphthyl, tetrahydronapthyl, indanyl, biphenyl.

The term “arylalkyl” refers to an aryl group as defined herein directly bonded to an alkyl group as defined herein which is then attached to the main structure at any carbon from alkyl group that results in the creation of a stable structure the rest of the molecule. e.g., —CH₂C₆H₅, —C₂H₅C₆H₅.

The term “heterocyclic ring” refers to a stable 3- to 15 membered ring radical which consists of carbon atoms and from one to five heteroatoms selected from the group consisting of nitrogen, phosphorus, oxygen and sulfur. For purposes of this invention, the heterocyclic ring radical may be a monocyclic, bicyclic or tricyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized; and the ring radical may be partially or fully saturated (i.e., heteroaromatic or heteroaryl aromatic). Examples of such heterocyclic ring radicals include, but are not limited to, azetidinyl, acridinyl, benzodioxolyl, benzodioxanyl, benzofurnyl, carbazolyl cinnolinyl dioxolanyl, indolizinyl, naphthyridinyl, perhydroazepinyl, phenazinyl, phenothiazinyl, phenoxazinyl, phthalazil, pyridyl, pteridinyl, purinyl, quinazolinyl, quinoxalinyl, quinolinyl, isoquinolinyl, tetrazoyl, imidazolyl tetrahydroisouinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazinyl, pyrimidinyl pyridazinyl, oxazolyl oxazolinyl oxasolidinyl, triazolyl, indanyl, isoxazolyl, isoxasolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl quinolyl, isoquinolyl, decahydroisoquinolyl, benzimidazolyl, thiadiazolyl, benzopyranyl, benzothiazolyl, benzooxazolyl, furyl, tetrahydrofurtyl, tetrahydropyranyl, thienyl, benzothienyl, thiamorpholinyl, thiamorpholinyl sulfoxide thiamorpholinyl sulfone, dioxaphospholanyl, oxadiazolyl, chromanyl, isochromanyl.

The term “heteroaryl” refers to heterocyclic ring radical as defined herein which are aromatic. The heteroaryl ring radical may be attached to the main structure at any heteroatom or carbon atom that results in the creation of a stable structure.

The heterocyclic ring radical may be attached to the main structure at any heteroatom or carbon atom that results in the creation of a stable structure.

The term “heteroarylalkyl” refers to heteroaryl ring radical as defined herein directly bonded to alkyl group. The heteroarylalkyl radical may be attached to the main structure at any carbon atom from alkyl group that results in the creation of a stable structure.

The term “heterocyclyl” refers to a heterocylic ring radical as defined herein. The heterocylyl ring radical may be attached to the main structure at any heteroatom or carbon atom that results in the creation of a stable structure.

The term “heterocyclylalkyl” refers to a heterocylic ring radical as defined herein directly bonded to alkyl group. The heterocyclylalkyl radical may be attached to the main structure at carbon atom in the alkyl group that results in the creation of a stable structure.

The term “carbonyl” refers to an oxygen atom bound to a carbon atom of the molecule by a double bond.

The term “halogen” refers to radicals of fluorine, chlorine, bromine and iodine.

Where the plural form of the word compounds, salts, polymorphs, hydrates, solvates and the like, is used herein, this is taken to mean also a single compound, salt, polymorph, isomer, hydrate, solvate or the like.

The compounds of this invention may contain one or more asymmetric centers, depending upon the location and nature of the various substituents desired. Asymmetric carbon atoms may be present in the (R) or (S) configuration, resulting in racemic mixtures in the case of a single asymmetric center, and diastereomeric mixtures in the case of multiple asymmetric centers. In certain instances, asymmetry may also be present due to restricted rotation about a given bond, for example, the central bond adjoining two substituted aromatic rings of the specified compounds. Substituents on a ring may also be present in either cis or trans form. It is intended that all such configurations (including enantiomers and diastereomers), are included within the scope of the present invention. Preferred compounds are those, which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques known in the art.

The present invention also relates to useful forms of the compounds as disclosed herein, such as pharmaceutically acceptable salts, co-precipitates, metabolites, hydrates, solvates and prodrugs of all the compounds of examples. The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19. Pharmaceutically acceptable salts include those obtained by reacting the main compound, functioning as a base, with an inorganic or organic acid to form a salt, for example, salts of hydrochloric acid, sulfuric acid, phosphoric acid, methane sulfonic acid, camphor sulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, magnesium, ammonium, and chorine salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

Representative salts of the compounds of this invention include the conventional non-toxic salts and the quaternary ammonium salts which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, chloride, bromide, iodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, sulfate, tartrate, thiocyanate, tosylate, and undecanoate.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, or butyl chlorides, bromides and iodides; dialkyl sulfates like dimethyl, diethyl, dibutyl sulfate, or diamyl sulfates, long chain halides such as decyl, lauryl, myristyl and strearyl chlorides, bromides and iodides, aralkyl halides like benzyl and phenethyl bromides and others.

A solvate for the purpose of this invention is a complex of a solvent and a compound of the invention in the solid state. Exemplary solvates would include, but are not limited to, complexes of a compound of the invention with ethanol or methanol. Hydrates are a specific form of solvate wherein the solvent is water.

The synthesis of the compounds listed above is described in International Patent Application No. PCT/EP2003/010377, published as WO 2004/029055 A1, and in International Patent Application No. PCT/US2007/024985, published as WO 2008/070150, both of which are hereby incorporated herein in their entirety by reference.

In accordance with another embodiment, the present invention relates to a 2,3-dihydroimidazo[1,2-c]quinazoline compound as defined herein, in particular 2-amino-N-[7-methoxy-8-(3-morpholin-4-ylpropoxy)-2,3-dihydroimidazo[1,2-c]quinazolin-5-yl]pyrimidine-5-carboxamide, or a physiologically acceptable salt, solvate, hydrate or stereoisomer thereof, as a sole agent, for the treatment of endometrial cancer (hereinafter abbreviated to “EC”), particularly 1st line, 2nd line, relapsed, refractory, type I or type II EC, or endometriosis.

In accordance a particular embodiment of any of the above aspects, or embodiments thereof, of the present invention, said cancer is endometrial cancer (hereinafter abbreviated to “EC”), particularly 1st line, 2nd line, relapsed, refractory, type I or type II EC, or endometriosis.

Combination Therapies

As mentioned supra, the present invention relates to combinations of:

a) a 2,3-dihydroimidazo[1,2-c]quinazoline compound as defined supra, or a physiologically acceptable salt, solvate, hydrate or stereoisomer thereof; or pharmaceutical compositions containing such a compound or a physiologically acceptable salt, solvate, hydrate or stereoisomer thereof;

and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent.

In a preferred embodiment, the invention encompasses combinations of:

a) 2-amino-N-[7-methoxy-8-(3-morpholin-4-ylpropoxy)-2,3-dihydroimidazo[1,2-c]quinazolin-5-yl]pyrimidine-5-carboxamide, or a physiologically acceptable salt, solvate, hydrate or stereoisomer thereof; or pharmaceutical compositions containing such a compound or a physiologically acceptable salt, solvate, hydrate or stereoisomer thereof;

and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent.

The compounds of this invention can be administered as the sole pharmaceutical agent or in combination with one or more other pharmaceutical agents (or “further active agents”) where the combination causes no unacceptable adverse effects. For example, the compounds of this invention can be combined with known anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agents, and the like, as well as with admixtures and combinations thereof.

The additional pharmaceutical agent or agents (or “further active agent”) can be, but are not limited to 131I-chTNT, abarelix, abiraterone, aclarubicin, adotrastuzumab emtansine, afatinib, aflibercept, aldesleukin, alemtuzumab, Alendronic acid, alitretinoin, altretamine, amifostine, aminoglutethimide, Hexyl aminolevulinate, amrubicin, amsacrine, anastrozole, ancestim, anethole dithiolethione, angiotensin II, antithrombin III, aprepitant, arcitumomab, arglabin, arsenic trioxide, asparaginase, axitinib, azacitidine, basiliximab, belotecan, bendamustine, belinostat, bevacizumab, bexarotene, bicalutamide, bisantrene, bleomycin, bortezomib, buserelin, bosutinib, brentuximab vedotin, busulfan, cabazitaxel, cabozantinib, calcium folinate, calcium levofolinate, capecitabine, capromab, carboplatin, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, copanlisib, crisantaspase, cyclophosphamide, cyproterone, cytarabine, dacarbazine, dactinomycin, darbepoetin alfa, dabrafenib, dasatinib, daunorubicin, decitabine, degarelix, denileukin diftitox, denosumab, depreotide, deslorelin, dexrazoxane, dibrospidium chloride, dianhydrogalactitol, diclofenac, docetaxel, dolasetron, doxifluridine, doxorubicin, doxorubicin+estrone, dronabinol, eculizumab, edrecolomab, elliptinium acetate, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (123I), iomeprol, ipilimumab, irinotecan, Itraconazole, ixabepilone, lanreotide, lapatinib, lasocholine, lenalidomide, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, nedaplatin, nelarabine, neridronic acid, nivolumabpentetreotide, nilotinib, nilutamide, nimorazole, nimotuzumab, nimustine, nitracrine, nivolumab, obinutuzumab, octreotide, ofatumumab, omacetaxine mepesuccinate, omeprazole, ondansetron, oprelvekin, orgotein, orilotimod, oxaliplatin, oxycodone, oxymetholone, ozogamicine, p53 gene therapy, paclitaxel, palifermin, palladium-103 seed, palonosetron, pamidronic acid, panitumumab, pantoprazole, pazopanib, pegaspargase, PEG-epoetin beta (methoxy PEG-epoetin beta), pembrolizumab, pegfilgrastim, peginterferon alfa-2b, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, romidepsin, romiplostim, romurtide, roniciclib, samarium (153Sm) lexidronam, sargramostim, satumomab, secretin, sipuleucel-T, sizofiran, sobuzoxane, sodium glycididazole, sorafenib, stanozolol, streptozocin, sunitinib, talaporfin, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin, or a combination thereof.

The additional pharmaceutical agent or agents (or “further active agent”) can be, but are not limited to aldesleukin, alendronic acid, alfaferone, alitretinoin, allopurinol, aloprim, aloxi, altretamine, aminoglutethimide, amifostine, amrubicin, amsacrine, anastrozole, anzmet, aranesp, arglabin, arsenic trioxide, aromasin, 5-azacytidine, azathioprine, BCG or tice BCG, bestatin, betamethasone acetate, betamethasone sodium phosphate, bexarotene, bleomycin sulfate, broxuridine, bortezomib, busulfan, calcitonin, campath, capecitabine, carboplatin, casodex, cefesone, celmoleukin, cerubidine, chlorambucil, cisplatin, cladribine, cladribine, clodronic acid, cyclophosphamide, cytarabine, dacarbazine, dactinomycin, DaunoXome, decadron, decadron phosphate, delestrogen, denileukin diftitox, depo-medrol, deslorelin, dexomethasone, dexrazoxane, diethylstilbestrol, diflucan, docetaxel, doxifluridine, doxorubicin, dronabinol, DW-166HC, eligard, elitek, ellence, emend, epirubicin, epoetin alfa, epogen, eptaplatin, ergamisol, estrace, estradiol, estramustine phosphate sodium, ethinyl estradiol, ethyol, etidronic acid, etopophos, etoposide, fadrozole, farston, filgrastim, finasteride, fligrastim, floxuridine, fluconazole, fludarabine, 5-fluorodeoxyuridine monophosphate, 5-fluorouracil (5-FU), fluoxymesterone, flutamide, formestane, fosteabine, fotemustine, fulvestrant, gammagard, gemcitabine, gemtuzumab, gleevec, gliadel, goserelin, granisetron HCl, herceptin, histrelin, hycamtin, hydrocortone, eyrthro-hydroxynonyladenine, hydroxyurea, ibritumomab tiuxetan, idarubicin, ifosfamide, interferon alpha, interferon-alpha 2, interferon alfa-2A, interferon alfa-2B, interferon alfa-n1, interferon alfa-n3, interferon beta, interferon gamma-1a, interleukin-2, intron A, iressa, irinotecan, kytril, lapatinib, lentinan sulphate, letrozole, leucovorin, leuprolide, leuprolide acetate, lenalidomide, levamisole, levofolinic acid calcium salt, levothroid, levoxyl, lomustine, lonidamine, marinol, mechlorethamine, mecobalamin, medroxyprogesterone acetate, megestrol acetate, melphalan, menest, 6-mercaptopurine, Mesna, methotrexate, metvix, miltefosine, minocycline, mitomycin C, mitotane, mitoxantrone, Modrenal, Myocet, nedaplatin, neulasta, neumega, neupogen, nilutamide, nolvadex, NSC-631570, OCT-43, octreotide, ondansetron HCl, orapred, oxaliplatin, paclitaxel, pediapred, pegaspargase, Pegasys, pentostatin, picibanil, pilocarpine HCl, pirarubicin, plicamycin, porfimer sodium, prednimustine, prednisolone, prednisone, premarin, procarbazine, procrit, refametinib (BAY 86-9766 (RDEA 119)), raltitrexed, rebif, rhenium-186 etidronate, rituximab, roferon-A, romurtide, salagen, sandostatin, sargramostim, semustine, sizofiran, sobuzoxane, solumedrol, sparfosic acid, stem-cell therapy, streptozocin, strontium-89 chloride, sunitinib, synthroid, tamoxifen, tamsulosin, tasonermin, tastolactone, taxotere, teceleukin, temozolomide, teniposide, testosterone propionate, testred, thioguanine, thiotepa, thyrotropin, tiludronic acid, topotecan, toremifene, tositumomab, trastuzumab, treosulfan, tretinoin, trexall, trimethylmelamine, trimetrexate, triptorelin acetate, triptorelin pamoate, UFT, uridine, valrubicin, vesnarinone, vinblastine, vincristine, vindesine, vinorelbine, virulizin, zinecard, zinostatin stimalamer, zofran, ABI-007, acolbifene, actimmune, affinitak, aminopterin, arzoxifene, asoprisnil, atamestane, atrasentan, BAY 43-9006 (sorafenib), avastin, CCI-779, CDC-501, celebrex, cetuximab, crisnatol, cyproterone acetate, decitabine, DN-101, doxorubicin-MTC, dSLIM, dutasteride, edotecarin, eflornithine, exatecan, fenretinide, histamine dihydrochloride, histrelin hydrogel implant, holmium-166 DOTMP, ibandronic acid, interferon gamma, intron-PEG, ixabepilone, keyhole limpet hemocyanin, L-651582, lanreotide, lasofoxifene, libra, lonafarnib, miproxifene, minodronate, MS-209, liposomal MTP-PE, MX-6, nafarelin, nemorubicin, neovastat, nolatrexed, oblimersen, onco-TCS, osidem, paclitaxel polyglutamate, pamidronate disodium, PN-401, QS-21, quazepam, R-1549, raloxifene, ranpirnase, 13-cis-retinoic acid, satraplatin, seocalcitol, T-138067, tarceva, taxoprexin, thalidomide, thymosin alpha 1, tiazofurine, tipifarnib, tirapazamine, TLK-286, toremifene, TransMID-107R, valspodar, vapreotide, vatalanib, verteporfin, vinflunine, Z-100, zoledronic acid or combinations thereof.

In accordance with an embodiment, the additional pharmaceutical agent or agents (or “further active agent”) is selected from the group consisting of: 131I-chTNT, abarelix, abiraterone, aclarubicin, aldesleukin, alemtuzumab, alitretinoin, altretamine, aminoglutethimide, amrubicin, amsacrine, anastrozole, arglabin, arsenic trioxide, asparaginase, azacitidine, basiliximab, BAY 1000394, refametinib (BAY 86-9766 (RDEA 119)), belotecan, bendamustine, bevacizumab, bexarotene, bicalutamide, bisantrene, bleomycin, bortezomib, buserelin, busulfan, cabazitaxel, calcium folinate, calcium levofolinate, capecitabine, carboplatin, carmofur, carmustine, catumaxomab, celecoxib, celmoleukin, cetuximab, chlorambucil, chlormadinone, chlormethine, cisplatin, cladribine, clodronic acid, clofarabine, crisantaspase, cyclophosphamide, cyproterone, cytarabine, dacarbazine, dactinomycin, darbepoetin alfa, dasatinib, daunorubicin, decitabine, degarelix, denileukin diftitox, denosumab, deslorelin, dibrospidium chloride, docetaxel, doxifluridine, doxorubicin, doxorubicin+estrone, eculizumab, edrecolomab, elliptinium acetate, eltrombopag, endostatin, enocitabine, epirubicin, epitiostanol, epoetin alfa, epoetin beta, eptaplatin, eribulin, erlotinib, estradiol, estramustine, etoposide, everolimus, exemestane, fadrozole, filgrastim, fludarabine, fluorouracil, flutamide, formestane, fotemustine, fulvestrant, gallium nitrate, ganirelix, gefitinib, gemcitabine, gemtuzumab, glutoxim, goserelin, histamine dihydrochloride, histrelin, hydroxycarbamide, I-125 seeds, ibandronic acid, ibritumomab tiuxetan, idarubicin, ifosfamide, imatinib, imiquimod, improsulfan, interferon alfa, interferon beta, interferon gamma, ipilimumab, irinotecan, ixabepilone, lanreotide, lapatinib, lenalidomide, lenograstim, lentinan, letrozole, leuprorelin, levamisole, lisuride, lobaplatin, lomustine, lonidamine, masoprocol, medroxyprogesterone, megestrol, melphalan, mepitiostane, mercaptopurine, methotrexate, methoxsalen, Methyl aminolevulinate, methyltestosterone, mifamurtide, miltefosine, miriplatin, mitobronitol, mitoguazone, mitolactol, mitomycin, mitotane, mitoxantrone, nedaplatin, nelarabine, nilotinib, nilutamide, nimotuzumab, nimustine, nitracrine, ofatumumab, omeprazole, oprelvekin, oxaliplatin, p53 gene therapy, paclitaxel, palifermin, palladium-103 seed, pamidronic acid, panitumumab, pazopanib, pegaspargase, PEG-epoetin beta (methoxy PEG-epoetin beta), pegfilgrastim, peginterferon alfa-2b, pemetrexed, pentazocine, pentostatin, peplomycin, perfosfamide, picibanil, pirarubicin, plerixafor, plicamycin, poliglusam, polyestradiol phosphate, polysaccharide-K, porfimer sodium, pralatrexate, prednimustine, procarbazine, quinagolide, raloxifene, raltitrexed, ranimustine, razoxane, regorafenib, risedronic acid, rituximab, romidepsin, romiplostim, sargramostim, sipuleucel-T, sizofiran, sobuzoxane, sodium glycididazole, sorafenib, streptozocin, sunitinib, talaporfin, tamibarotene, tamoxifen, tasonermin, teceleukin, tegafur, tegafur+gimeracil+oteracil, temoporfin, temozolomide, temsirolimus, teniposide, testosterone, tetrofosmin, thalidomide, thiotepa, thymalfasin, tioguanine, tocilizumab, topotecan, toremifene, tositumomab, trabectedin, trastuzumab, treosulfan, tretinoin, trilostane, triptorelin, trofosfamide, tryptophan, ubenimex, valrubicin, vandetanib, vapreotide, vemurafenib, vinblastine, vincristine, vindesine, vinflunine, vinorelbine, vorinostat, vorozole, yttrium-90 glass microspheres, zinostatin, zinostatin stimalamer, zoledronic acid, zorubicin.

The additional pharmaceutical agent can also be gemcitabine, paclitaxel, cisplatin, carboplatin, sodium butyrate, 5-FU, doxirubicin, tamoxifen, etoposide, trastumazab, gefitinib, intron A, rapamycin, 17-AAG, U0126, insulin, an insulin derivative, a PPAR ligand, a sulfonylurea drug, an α-glucosidase inhibitor, a biguanide, a PTP-1B inhibitor, a DPP-IV inhibitor, a 11-beta-HSD inhibitor, GLP-1, a GLP-1 derivative, GIP, a GIP derivative, PACAP, a PACAP derivative, secretin or a secretin derivative.

Optional anti-hyper-proliferative agents which can be added to the composition include but are not limited to compounds listed on the cancer chemotherapy drug regimens in the 11^{th} Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use with the composition of the invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyl adenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine.

Other anti-hyper-proliferative agents suitable for use with the composition of the invention include but are not limited to other anti-cancer agents such as epothilone and its derivatives, irinotecan, raloxifen and topotecan.

Generally, the use of cytotoxic and/or cytostatic agents in combination with a compound or composition of the present invention will serve to:

(1) yield better efficacy in reducing the growth of a tumor or even eliminate the tumor as compared to administration of either agent alone,

(2) provide for the administration of lesser amounts of the administered chemotherapeutic agents,

(3) provide for a chemotherapeutic treatment that is better tolerated in the patient with fewer deleterious pharmacological complications than observed with single agent chemotherapies and certain other combined therapies,

(4) provide for treating a broader spectrum of different cancer types in mammals, especially humans,

(5) provide for a higher response rate among treated patients,

(6) provide for a longer survival time among treated patients compared to standard chemotherapy treatments,

(7) provide a longer time for tumor progression, and/or

(8) yield efficacy and tolerability results at least as good as those of the agents used alone, compared to known instances where other cancer agent combinations produce antagonistic effects.

In accordance with an embodiment, the invention relates to combinations wherein said 2,3-dihydroimidazo[1,2-c]quinazoline compound is 2-amino-N-[7-methoxy-8-(3-morpholin-4-ylpropoxy)-2,3-dihydroimidazo[1,2-c]quinazolin-5-yl]pyrimidine-5-carboxamide.

In accordance with an embodiment, the invention relates to combinations wherein said 2,3-dihydroimidazo[1,2-c]quinazoline compound is 2-amino-N-[7-methoxy-8-(3-morpholin-4-ylpropoxy)-2,3-dihydroimidazo[1,2-c]quinazolin-5-yl]pyrimidine-5-carboxamide dihydrochloride.

Pharmaceutical Compositions of the Compounds of the Invention

As mentioned supra, the present invention relates to pharmaceutical compositions:
comprising a 2,3-dihydroimidazo[1,2-c]quinazoline compound, or a physiologically acceptable salt, solvate, hydrate or stereoisomer thereof, as a sole active agent, for the treatment of endometrial cancer (hereinafter abbreviated to “EC”), particularly 1st line, 2nd line, relapsed, refractory, type I or type II EC, or endometriosis, and
comprising a pharmaceutical composition which comprises a combination of:
   a) a 2,3-dihydroimidazo[1,2-c]quinazoline compound, or a physiologically acceptable salt, solvate, hydrate or stereoisomer thereof; and
   b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent.

In accordance with another embodiment, the present invention relates to pharmaceutical compositions which comprise a 2,3-dihydroimidazo[1,2-c]quinazoline compound as defined herein, in particular 2-amino-N-[7-methoxy-8-(3-morpholin-4-ylpropoxy)-2,3-dihydroimidazo[1,2-c]quinazolin-5-yl]pyrimidine-5-carboxamide, or a physiologically acceptable salt, solvate, hydrate or stereoisomer thereof, as a sole agent, for the treatment of endometrial cancer (hereinafter abbreviated to “EC”), particularly 1st line, 2nd line, relapsed, refractory, type I or type II EC, or endometriosis.

In accordance with another embodiment, the present invention relates to pharmaceutical compositions which comprise 2-amino-N-[7-methoxy-8-(3-morpholin-4-ylpropoxy)-2,3-dihydroimidazo[1,2-c]quinazolin-5-yl]pyrimidine-5-carboxamide dihydrochloride, as a sole agent, for the treatment of endometrial cancer (hereinafter abbreviated to “EC”), particularly 1st line, 2nd line, relapsed, refractory, type I or type II EC, or endometriosis.

In accordance a particular embodiment of any of the above aspects, or embodiments thereof, of the present invention, said cancer is endometrial cancer (hereinafter abbreviated to “EC”), particularly 1st line, 2nd line, relapsed, refractory, type I or type II EC, or endometriosis.

Said pharmaceutical compositions contain one or more compounds. These compositions can be utilized to achieve the desired pharmacological effect by administration to a patient in need thereof. A patient, for the purpose of this invention, is a mammal, including a human, in need of treatment for the particular condition or disease. Therefore, the present invention includes pharmaceutical compositions that are comprised of a pharmaceutically acceptable carrier and a pharmaceutically effective amount of a compound, or salt thereof, of the present invention. A pharmaceutically acceptable carrier is preferably a carrier that is relatively non-toxic and innocuous to a patient at concentrations consistent with effective activity of the active agent so that any side effects ascribable to the carrier do not vitiate the beneficial effects of the active agent. A pharmaceutically effective amount of compound is preferably that amount which produces a result or exerts an influence on the particular condition being treated. The compounds of the present invention can be administered with pharmaceutically-acceptable carriers well known in the art using any effective conventional dosage unit forms, including immediate, slow and timed release preparations, orally, parenterally, topically, nasally, ophthalmically, optically, sublingually, rectally, vaginally, and the like.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule that can be of the ordinary hard- or soft-shelled gelatin type containing, for example, surfactants, lubricants, and inert fillers such as lactose, sucrose, calcium phosphate, and corn starch.

In another embodiment, the compounds of this invention may be tableted with conventional tablet bases such as lactose, sucrose and cornstarch in combination with binders such as acacia, corn starch or gelatin, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active agent in admixture with a dispersing or wetting agent, a suspending agent and one or more preservatives. Suitable dispersing or wetting agents and suspending agents are exemplified by those already mentioned above. Additional excipients, for example those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions may be formulated by suspending the active agent in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavoring and coloring agents.

The compounds of this invention may also be administered parenterally, that is, subcutaneously, intravenously, intraocularly, intrasynovially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active agent in solution. Preservatives and buffers may also be used advantageously. In order to minimize or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations that are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized. Such ingredients and procedures include those described in the following references, each of which is incorporated herein by reference: Powell, M. F. et al, “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al, “Excipients and Their Use in Injectable Products” PDA Journal of Pharmaceutical Science & Technology 1997, 51(4), 166-171.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);

alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);

adsorbents (examples include but are not limited to powdered cellulose and activated charcoal);

aerosol propellants (examples include but are not limited to carbon dioxide, CCl₂F₂, F₂ClC—CClF₂ and CClF₃)

air displacement agents (examples include but are not limited to nitrogen and argon);

antifungal preservatives (examples include but are not limited to benzoic acid, butylparaben, ethylparaben, methylparaben, propylparaben, sodium benzoate);

antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);

antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

binding materials (examples include but are not limited to block polymers, natural and synthetic rubber, polyacrylates, polyurethanes, silicones, polysiloxanes and styrene-butadiene copolymers);

buffering agents (examples include but are not limited to potassium metaphosphate, dipotassium phosphate, sodium acetate, sodium citrate anhydrous and sodium citrate dihydrate)

carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)

chelating agents (examples include but are not limited to edetate disodium and edetic acid)

colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);

clarifying agents (examples include but are not limited to bentonite);

emulsifying agents (examples include but are not limited to acacia, cetomacrogol, cetyl alcohol, glyceryl monostearate, lecithin, sorbitan monooleate, polyoxyethylene 50 monostearate);

encapsulating agents (examples include but are not limited to gelatin and cellulose acetate phthalate)

flavorants (examples include but are not limited to anise oil, cinnamon oil, cocoa, menthol, orange oil, peppermint oil and vanillin);

humectants (examples include but are not limited to glycerol, propylene glycol and sorbitol);

levigating agents (examples include but are not limited to mineral oil and glycerin);

oils (examples include but are not limited to arachis oil, mineral oil, olive oil, peanut oil, sesame oil and vegetable oil);

ointment bases (examples include but are not limited to lanolin, hydrophilic ointment, polyethylene glycol ointment, petrolatum, hydrophilic petrolatum, white ointment, yellow ointment, and rose water ointment);

penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)

plasticizers (examples include but are not limited to diethyl phthalate and glycerol);

solvents (examples include but are not limited to ethanol, corn oil, cottonseed oil, glycerol, isopropanol, mineral oil, oleic acid, peanut oil, purified water, water for injection, sterile water for injection and sterile water for irrigation);

stiffening agents (examples include but are not limited to cetyl alcohol, cetyl esters wax, microcrystalline wax, paraffin, stearyl alcohol, white wax and yellow wax);

suppository bases (examples include but are not limited to cocoa butter and polyethylene glycols (mixtures));

surfactants (examples include but are not limited to benzalkonium chloride, nonoxynol 10, oxtoxynol 9, polysorbate 80, sodium lauryl sulfate and sorbitan mono-palmitate);

suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

sweetening agents (examples include but are not limited to aspartame, dextrose, glycerol, mannitol, propylene glycol, saccharin sodium, sorbitol and sucrose);

tablet anti-adherents (examples include but are not limited to magnesium stearate and talc);

tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);

tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);

tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);

tablet direct compression excipients (examples include but are not limited to dibasic calcium phosphate);

tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);

tablet glidants (examples include but are not limited to colloidal silica, corn starch and talc);

tablet lubricants (examples include but are not limited to calcium stearate, magnesium stearate, mineral oil, stearic acid and zinc stearate);

tablet/capsule opaquants (examples include but are not limited to titanium dioxide);

tablet polishing agents (examples include but are not limited to carnuba wax and white wax);

thickening agents (examples include but are not limited to beeswax, cetyl alcohol and paraffin);

tonicity agents (examples include but are not limited to dextrose and sodium chloride);

viscosity increasing agents (examples include but are not limited to alginic acid, bentonite, carbomers, carboxymethylcellulose sodium, methylcellulose, polyvinyl pyrrolidone, sodium alginate and tragacanth); and

wetting agents (examples include but are not limited to heptadecaethylene oxycetanol, lecithins, sorbitol monooleate, polyoxyethylene sorbitol monooleate, and polyoxyethylene stearate).

Pharmaceutical compositions according to the present invention can be illustrated as follows:

Sterile IV Solution: A 5 mg/mL solution of the desired compound of this invention can be made using sterile, injectable water, and the pH is adjusted if necessary. The solution is diluted for administration to 1-2 mg/mL with sterile 5% dextrose and is administered as an IV infusion over about 60 minutes.

Lyophilized powder for IV administration: A sterile preparation can be prepared with (i) 100-1000 mg of the desired compound of this invention as a lypholized powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 mg/mL, and is administered either IV bolus or by IV infusion over 15-60 minutes.

Intramuscular suspension: The following solution or suspension can be prepared, for intramuscular injection:

50 mg/mL of the desired, water-insoluble compound of this invention

5 mg/mL sodium carboxymethylcellulose

4 mg/mL TWEEN 80

9 mg/mL sodium chloride

9 mg/mL benzyl alcohol

Hard Shell Capsules: A large number of unit capsules are prepared by filling standard two-piece hard galantine capsules each with 100 mg of powdered active agent, 150 mg of lactose, 50 mg of cellulose and 6 mg of magnesium stearate.

Soft Gelatin Capsules: A mixture of active agent in a digestible oil such as soybean oil, cottonseed oil or olive oil is prepared and injected by means of a positive displacement pump into molten gelatin to form soft gelatin capsules containing 100 mg of the active agent. The capsules are washed and dried. The active agent can be dissolved in a mixture of polyethylene glycol, glycerin and sorbitol to prepare a water miscible medicine mix.

Tablets: A large number of tablets are prepared by conventional procedures so that the dosage unit is 100 mg of active agent, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules: These are solid oral dosage forms made by conventional and novel processes. These units are taken orally without water for immediate dissolution and delivery of the medication. The active agent is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Method of Treating Endometrial Cancer (Hereinafter Abbreviated to “EC”), Particularly 1st Line, 2nd Line, Relapsed, Refractory, Type I or Type II EC, or Endometriosis

The present invention also relates to a method of treating or prophylaxis of endometrial cancer (hereinafter abbreviated to “EC”), particularly 1st line, 2nd line, relapsed, refractory, type I or type II EC, or endometriosis, in a mammal, said method comprising administering a 2,3-dihydroimidazo[1,2-c]quinazoline compound as defined herein, or a pharmaceutical composition containing same, as a sole active agent, or administering a combination of a) said compound or a pharmaceutical composition containing said compound and b) one or more further active agents as defined herein.

In accordance a particular embodiment of any of the above aspects, or embodiments thereof, of the present invention, said cancer is endometrial cancer (hereinafter abbreviated to “EC”), particularly 1st line, 2nd line, relapsed, refractory, type I or type II EC, or endometriosis.

The embodiments of the methods of treating or prophylaxis of cancer, e.g. endometrial cancer (hereinafter abbreviated to “EC”), particularly 1st line, 2nd line, relapsed, refractory, type I or type II EC, or endometriosis, as defined supra, are as described in the embodiments of the use of the compounds/combinations, as described supra.

The present invention relates to a method for using the compounds of the present invention and compositions thereof, to treat mammalian endometrial cancer (hereinafter abbreviated to “EC”), particularly 1st line, 2nd line, relapsed, refractory, type I or type II EC, or endometriosis. Compounds can be utilized to inhibit, block, reduce, decrease, etc., cell proliferation and/or cell division, and/or produce apoptosis, in the treatment or prophylaxis of endometrial cancer (hereinafter abbreviated to “EC”), particularly 1st line, 2nd line, relapsed, refractory, type I or type II EC, or endometriosis. This method comprises administering to a mammal in need thereof, including a human, an amount of a compound or combination of this invention, or a pharmaceutically acceptable salt, isomer, polymorph, metabolite, hydrate, solvate or ester thereof; etc. which is effective for the treatment or prophylaxis of endometrial cancer (hereinafter abbreviated to “EC”), particularly 1st line, 2nd line, relapsed, refractory, type I or type II EC, or endometriosis.

Examples of endometrial cancer include, but not limited to type I EC (estrogen-dependent and/or progesterone-dependent with endometrioid histology) and type II EC, or endometriosis (hormone-independent poorly differentiated endometrioid, clear cell and serous carcinomas).

This disorder has been well characterized in humans, but also exists with a similar etiology in other mammals, and they can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

The present invention relates to a method for using single agent and the combinations of the present invention, in the treatment or prophylaxis of a cancer, particularly endometrial cancer (hereinafter abbreviated to “EC”), particularly 1st line, 2nd line, relapsed, refractory, type I or type II EC, or endometriosis. Single agent and Combinations can be utilized to inhibit, block, reduce, decrease, etc., cell proliferation and/or cell division, and/or produce apoptosis, in the treatment or prophylaxis of cancer, in particular EC (hereinafter abbreviated to “EC”), particularly 1st line, 2nd line, relapsed, refractory, type I or type II EC, or endometriosis. This method comprises administering to a mammal in need thereof, including a human, an amount of a combination of this invention, or a pharmaceutically acceptable salt, isomer, polymorph, metabolite, hydrate, solvate or ester thereof; etc. which is effective for the treatment or prophylaxis of cancer, in particular EC, particularly 1st line, 2nd line, relapsed, refractory, type I or type II EC, or endometriosis.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

Dose and Administration

Based upon standard laboratory techniques known to evaluate compounds useful for the treatment or prophylaxis of cancer, in particular endometrial cancer (EC), particularly 1st line, 2nd line, relapsed, refractory, type I or type II EC, or endometriosis, by standard toxicity tests and by standard pharmacological assays for the determination of treatment of the conditions identified above in mammals, and by comparison of these results with the results of known medicaments that are used to treat these conditions, the effective dosage of the combinations of this invention can readily be determined for treatment of the indication. The amount of the active ingredient to be administered in the treatment of the condition can vary widely according to such considerations as the particular combination and dosage unit employed, the mode of administration, the period of treatment, the age and sex of the patient treated, and the nature and extent of the condition treated.

Dose and Administration

Based upon standard laboratory techniques known to evaluate compounds useful for the treatment or prophylaxis of endometrial cancer (hereinafter abbreviated to “EC”), particularly 1st line, 2nd line, relapsed, refractory, type I or type II EC, or endometriosis, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of the indication. The amount of the active agent to be administered in the treatment of the condition can vary widely according to such considerations as the particular compound and dosage unit employed, the mode of administration, the period of treatment, the age and sex of the patient treated, and the nature and extent of the condition treated.

The total amount of the active agent to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1,500 mg of active agent, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Biomarkers:

Biomarkers used for patient stratification are e.g. the loss of tumor suppressor PTEN or FBXW7,
either alone or in combination with another form of PI3K pathway activation selected from perturbation of any of the following alone or in combination: mutation in PIK3CA, PIK3CB, PIK3CD, PIK3CG, PIK3R1, PIK3R2, PIK3R3, PIK3R4, PIK3R5, FGFR1, FGFR2, FGFR3 and/or FGFR4; PTEN loss and alteration of PIK3CA, PIK3CB, PIK3CD, PIK3CG, PIK3R1, PIK3R2, PIK3R3, PIK3R4, PIK3R5, FGFR1, FGFR2, FGFR3 and/or FGFR4, which may be measured at either the protein level, mRNA level, or DNA level,

for predicting the sensitivity and/or resistance of a patient with endometrial cancer (hereinafter abbreviated to “EC”), particularly 1st line, 2nd line, relapsed, refractory, type I or type II EC, or endometriosis, to a 2,3-dihydroimidazo[1,2-c]quinazoline compound as defined herein, thus providing rationale-based dosage as defined herein to overcome said resistance of a patient with endometrial cancer (hereinafter abbreviated to “EC”), particularly 1st line, 2nd line, relapsed, refractory, type I or type II EC, or endometriosis, to a 2,3-dihydroimidazo[1,2-c]quinazoline compound as defined herein (patient stratification).

Compounds Used

Throughout the whole of this text, including in the Examples which follow:

1. “compound of formula I” refers to 2-amino-N-[7-methoxy-8-(3-morpholin-4-ylpropoxy)-2,3-dihydroimidazo[1,2-c]quinazolin-5-yl]pyrimidine-5-carboxamide, of structure:

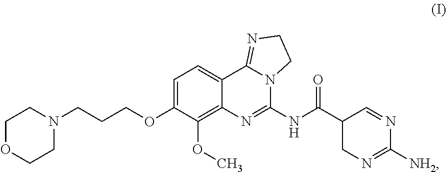 [SMILES: COc1c(OCCCN2CCOCC2)ccc2c1N=C(NC(=O)C1C=NC(N)=NC1)N1CCN=C21]

or a solvate, hydrate or stereoisomer thereof.

2. “compound A” refers to 2-amino-N-[7-methoxy-8-(3-morpholin-4-ylpropoxy)-2,3-dihydroimidazo[1,2-c]quinazolin-5-yl]pyrimidine-5-carboxamide dihydrochloride, of structure:

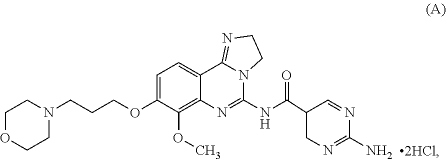 [SMILES: COc1c(OCCCN2CCOCC2)ccc2c1N=C(NC(=O)C1C=NC(N)=NC1)N1CCN=C21]

or a solvate, hydrate or stereoisomer thereof.

The synthesis of compound A is described in European patent application number EP 11 161 111.7, and in PCT application number PCT/EP2012/055600 published under WO 2012/136553, both of which are hereby incorporated herein in their entirety by reference.

Synthesis of compound A:

To a suspension of the compound of formula I (400 g) in water (1.1 L) at room temperature was added a 32% aqueous 32% (aqueous) hydrochloric acid solution iswith stirring dosed at room temperature to a suspension of 400 g of the compound of formula (I) in 1.1 L water until a pH of 3-4 is was reached. Additional 90 mL water (90 mL) and 32% hydrochloric acid are were added until a pH of 1.8 to 2.0 is was attained. E160 mL ethanol (160 mL) are dosed into was added to the mixture, followed by seed crystals. After stirring for 30 minutes, 1740 g additional ethanol (2.2 L) are dosed within 5 h was added into the mixture over 5 h, which is and the resulting mixture was subsequently stirred for 1 h. The suspension is filtered and the residue is washed first with a mixture of 130 g water and 215 g ethanol, secondly with a mixture of 80 g water and 255 g ethanol and then with 320 g pure ethanol. The filter cake is dried at 40° C. under vacuum to yield 457 g product (99% of theory).

Further Method of Preparation of Compound “A”

To a suspension of 366 g of compound of formula (I) in 1015 g water, 183 g of an aqueous hydrochloric acid solution (32%) were added while maintaining the temperature at 20° C. (+−2°) until a pH of 3 to 4 was reached. The resulting mixture was stirred at room temperature for more than 10 min. filtered and the filtercake washed with additional 82 g of water. The filtrate was adjusted to pH 1.8 to 2.0 using aqueous hydrochloric acid solution (32%). The mixture was stirred for 10 min. at room temperature, 146 g of ethanol (100%) were added and stirred for another 10 min. 1 g of seed crystals were added, followed by 1592 g ethanol within 5 h. The resulting substance was removed by filtration, washed with a water-ethanol mixture and dried in vacuo to give 410 g (97%) of compound A of a purity >99% according to HPLC.

### EXAMPLES

The invention is demonstrated in the following examples which are not meant to limit the invention in any way:

Materials and Methods:

In vitro proliferation assays: Cell proliferation is determined using the Cell Titer-Glo luminescent cell viability kit from Promega (Cat. #G7573) after 72 hours exposure to BAY 1082439. Briefly, cells were plated at 1000-5000 cells/well of 96-well plates (based on cell lines) in 90 μL of growth medium. For each cell line assayed, cells were plated into a separate plate for determination of luminescence at the t=0 hours and t=72 hour time points. Following overnight incubation at 37° C., luminescence values for the t=0 samples were determined by adding 90 μL of Cell Titer-Glo solution per well, transferring the plates to an orbital shaker for 10 minutes at room temperature, and then reading the plates on a Wallac Victor2 1420 Multilabel HTS Counter using the luminometry window (maximum light detection is measured at 428 nM). Dose plates for t=72 hour time points were treated with compounds diluted into growth medium in a final volume of 100 μL. Cells were then incubated for 72 hours at 37° C. Luminescence values for the t=72 hour samples were determined by adding 100 μL of Promega CellTiter-Glo solution, placing the cells on a shaker for 10 minutes at room temperature, and then reading the luminescence using a Victor luminometer. For data processing, t=0 values are subtracted from those determined for the t=72 hour time points, for both the treated and untreated samples. Percent differences in luminescence between drug treated and controls are used to determine percent inhibition of growth.

The in vivo efficacy was evaluated in tumor xenograft models in nude mice with established human tumor cell lines at the MTD and sub-MTD dosages. Tumor cells were cultivated according to ATCC protocols in recommended media contained 10% FCS. Cells were harvested for transplantation in a subconfluent (70%) state. The number of cells for inoculation was indicated in Table 1. The volume of implantation was 100 μl for mice. When the tumors were approximately in size of 25-50 mm², the animals were randomized to treatment and control groups and treatment was started. Treatment of each animal was based on individual body weight. The optimal formulation, application route and schedule were used for each compound (see table 2). Oral administration (p.o.) was carried out via a gastric tube. The oral application volumes were 10 ml/kg and the intravenous application volumes were 10 ml/kg. Tumor area (product of the longest diameter and its perpendicular) using a calliper. The animal body weight was monitored as a measure for treatment-related toxicity. Measurement of tumor area and body weight was performed 2-3 times weekly. T/C ratios (Treatment/Control) were calculated with final tumor areas. Treatment responses were evaluated by means of the clinically-used RECIST criteria (complete response, partial response, stable disease and progressive disease) and response rates were calculated accordingly (RR=number of animals with complete and partial response).

TABLE 1
Tumor models used for assessment of compound A (copanlisib)
and FGFR inhibitors in endometrial tumor models in vivo.

| Tumor model | Mode of Implantation |
|---|---|
| HEC-1-A | s.c. implantation of 3 × 10⁶ cells suspended in 50% |
| | Matrigel into the inguinal region of female mice |
| HEC-1-B | s.c. implantation of 3 × 10⁶ cells suspended in 50% |
| | Matrigel into the inguinal region of female mice |
| MFE 280 | s.c. implantation of 1 × 10⁶ cells suspended in 50% |
| | Matrigel into the inguinal region of female mice |

TABLE 2
Formulations, application route and schedules
used in the in vivo studies.

| | | Appli- | Appli- |
|---|---|---|---|
| | | cation | cation |
| Drug | Formulation | route | schedule |
| Compound A | 5% Mannitol/0.9% NaCl | i.v. | Q2D |
| Doxorubicin | 0.9% NaCl | i.p. | Q14D |
| Compound B | 10% EtOH, 40% Solutol, 50% | p.o. | QD |
| | water (~2% HCl [2M]) | | |

The invention is demonstrated in the following examples which are not meant to limit the invention in any way:

### Example 1

### In Vitro Anti-Proliferative Activity of Compound A (Copanlisib)

TABLE 3
Single agent activity of compound A (copanlisib) in endometrial
tumor cell lines representing varied histological and
molecular features of human endometrial cancer.

| | | | His- |
|---|---|---|---|
| Cell | | | tological |
| Line | IC50 (M) | Molecular features | Subtype |
| RUCA | 1.37E−08 | PTEN^{del}, ER+ | Type I |
| ECC-1 | 1.61E−7 | PTEN^{del}, PIK3R1^{mut}, ER+, PR+, | |
| Ishikawa | 3.73E−07 | PTEN^{del}, PIK3R1^{mut}, ER+, PR+ | |
| HEC-50 | 6.46E−08 | PIK3R1^{mut}, KRAS^{mut} | |
| RL95-2 | 1.44E−07 | PIK3R1^{mut}/PTEN^{del}/NF2/BRCA2/ | |
| KLE | <6.86E−09 | FBXW7^{mut} | |
| HEC-1B | <6.86E−09 | PIK3CA^{mut}, PIK3R2^{mut}, | Type II |
| | | PTEN^{Loss}(protein) and KRAS^{mut} | |
| AN3CA | <6.86E−09 | PTEN^{del}, FBXW7, | |
| | | FGFR2^{N549K, K310R} | |
| HEC-1A | <6.86E−09 | PIK3CA^{G1049R}, PIK3R2^{mut}, | |
| | | KRAS^{mut} | |
| MFE 280 | <6.86E−09 | PIK3CA^{G1047Y}, RB^{del}, | |
| | | FGFR2^{S252W} | |
| MFE 296 | <6.86E−09 | PTEN^{del}, FGFR2^{N549K}, UTX^{del,} | |

Compound A (copanlisib) showed potent activity (IC₅₀ below 50 nM) in both type I/hormone-dependent (RUCA) and type II/hormone-independent (KLE, HEC-1A, HEC-1B, AN3CA, MFE280 and MFE 296) endometrial tumor cell lines. In addition, tumors with activating mutation(s) in PIK3CA, PIK3R1, PIK3R2, FGFR2 and/or loss of tumor suppressor PTEN or FBXW7 are sensitive to PI3K inhibition by copanlisib. These molecules could be used as biomarker(s) (one or combination of multiple markers) for predicting the sensitivity of tumors to copanlisib.

### Example 2

### In Vivo Efficacy of Copanlisib in HEC-1A, HEC-1B and MFE-280 Endometrial Xenograft Tumor Models

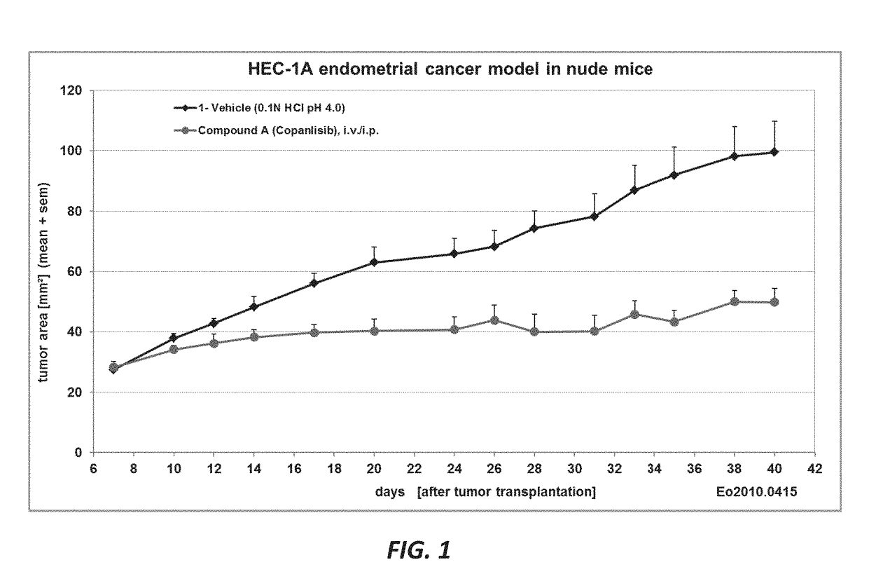

FIG. 1. Compound A (copanlisib) was tested HEC-1A, a tumor model bearing PIK3CA^{G1049R}, PIK3R2^{mut}, KRAS^{mut}. Treatment with 14 mg/kg Q2D i.v. Compound A (copanlisib) was efficacious with final tumor weight T/C of 0.36. However all animals showed progressive tumor growth (Table 4). The activating KRAS mutation in HEC-1A tumor cells could be the reason for lack of tumor responses, as it provides PI3K-independent survival signaling via MAPK pathway. Treatment with Compound A was generally well tolerated with a 5.1% maximum body weight loss during the treatment.

TABLE 4
Summary of Compound A activity and tolerability
in HEC-1A xenograft tumor model.

| | | | | Max. Body | |
|---|---|---|---|---|---|
| | Dose (mg/kg) | T/C^{a} | T/C | weight loss^{b} | Response |
| Compound | and Schedule | weight | area | (%) | rate^{c} |
| Vehicle | 10 ml/kg | 1.00 | 1.00 | / | 0% |
| Compound | 14 mg/kg Q2D | 0.36 | 0.50 | −5.1 | 0% |
| A | | | | | |

^{a}T/C = Treatment/Control ratio, Calculated from mean tumor areas or final tumor weights at the study end.
^{b}Body Weight Loss: the maximum mean body weight loss expressed as a percent of the starting weight of the animal. Weight loss greater than 20% is considered toxic.
^{c}Response: PD = progressive disease, the number of tumors exhibiting >20% tumor increase; SD = stable disease, the number of tumors exhibiting <30% tumor shrinkage and <20% tumor increase; PR = partial response, the number of tumors exhibiting >30% tumor shrinkage; CR = complete response, the number of not measureable tumors.

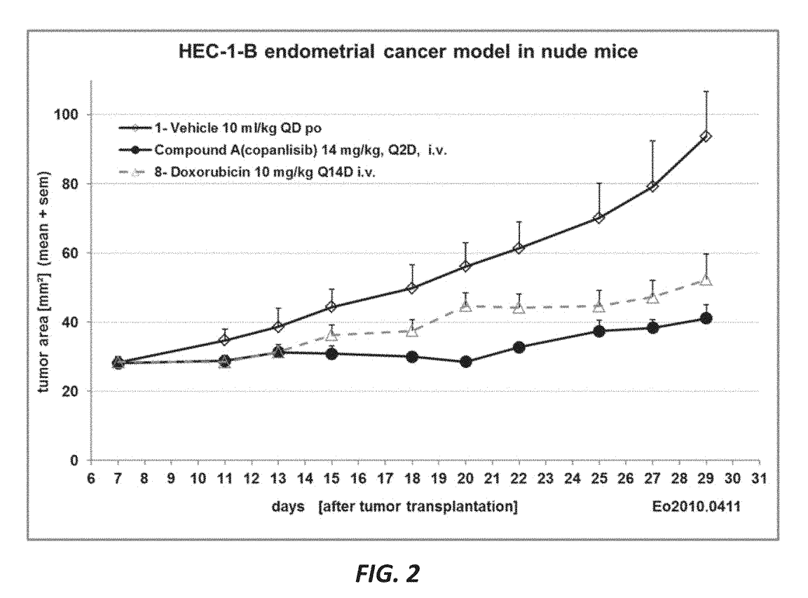

FIG. 2. In vivo efficacy of copanlisib in HEC-1B endometrial xenograft tumor model. Treatment with 14 mg/kg Q2D i.v. Compound A (copanlisib) was efficacious in HEC-1B xenograft tumor model with final tumor weight T/C of 0.28 compared to a T/C value of 0.48 for Doxorubicin (Table 5), a standard of care (SoC) therapy for endometrial cancer. Again, the activating KRAS mutation could be the reason for lack of tumor responses. Treatment with Compound A was generally well tolerated with a 1.4% maximum body weight loss during the treatment.

TABLE 5
Activity and tolerability of compound A (copanlisib)
in HEC-1B xenograft tumor model.

| | | | | Max. Body | | | |
|---|---|---|---|---|---|---|---|
| | Dose (mg/kg) | T/C^{a} | T/C | weight loss^{b} | Response | | |
| Compound | and Schedule | weight | area | (%) | rate^{c} | SD | PD |
| Vehicle | 10 ml/kg | 1.00 | 1.00 | / | 0% | 0 | 7 |
| Copanlisib | 14 mg/kg Q2D | 0.28 | 0.44 | −1.4 | 0% | 1 | 6 |
| dihydrochloride | | | | | | | |
| Doxorubicin | 10 mg/kg Q14D | 0.48 | 0.56 | −2.8 | 0% | 2 | 6 |

^{a}T/C = Treatment/Control ratio, Calculated from mean tumor areas or final tumor weights at the study end.
^{b}Body Weight Loss: the maximum mean body weight loss expressed as a percent of the starting weight of the animal. Weight loss greater than 20% is considered toxic.
^{c}Response: PD = progressive disease, the number of tumors exhibiting >20% tumor increase; SD = stable disease, the number of tumors exhibiting <30% tumor shrinkage and <20% tumor increase; PR = partial response, the number of tumors exhibiting >30% tumor shrinkage; CR = complete response, the number of not measureable tumors.

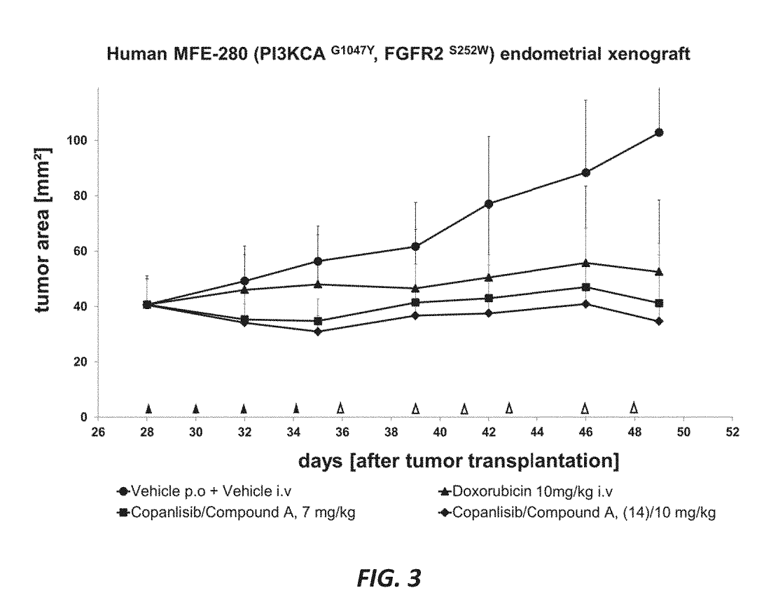

FIG. 3. Compound A (copanlisib) was tested MFE-280, a tumor model bearing PIK3CA^{G1047Y}, RB^{del}, FGFR2^{S252W}. Treatment with 14 mg/kg i.v. Compound A (copanlisib) for 5 times and then 10 mg/kg for 5 times at the schedule indicated with triangles was efficacious with final tumor size T/C of 0.34 and tumor weight T/C of 0.16.

TABLE 6
Activity of compound A (copanlisib)
in MFE-280 xenograft tumor model.

| | T/C^{a} | T/C | | | | | |
|---|---|---|---|---|---|---|---|
| Treatment | (tumor | (tumor | RR^{b} | CR^{b} | PR^{b} | SD^{b} | PD^{b} |
| group | area) | weight) | (%) | (%) | (%) | (%) | (%) |
| Vehicle | 1.00 | 1.00 | 0 | 0 | 0 | 0 | 100% |
| Copanlisib | 0.34 | 0.16 | 37.5 | 0 | 37.5 | 62.5 | 0 |
| (14)10 mg/kg | | | | | | | |
| Copanlisib | 0.40 | 0.24 | 25 | 0 | 25 | 50 | 25 |
| 7 mg/kg | | | | | | | |
| doxorubicin | 0.51 | 0.39 | 12.5 | 0 | 12.5 | 50 | 37.5 |

^{a}T/C = Treatment/Control ratio, Calculated from mean tumor areas or final tumor weights at the study end.
^{b}Response: PD = progressive disease, the number of tumors exhibiting >20% tumor increase; SD = stable disease, the number of tumors exhibiting <30% tumor shrinkage and <20% tumor increase; PR = partial response, the number of tumors exhibiting >30% tumor shrinkage; CR = complete response, the number of not measureable tumors.

### Example 3

### Clinical Benefit of PI3K Inhibitor Compound A (Copanlisib) in Endometrial Cancer Patients

In a phase I dose escalation study, subjects were treated with Compound A (copanlisib) administered intravenously over 60 minutes on days 1, 8, and 15 of every 28 day cycle. Seventeen subjects were treated in 5 dose escalation cohorts (0.1, 0.2, 0.4, 0.8, and 1.2 mg/kg), and the maximum tolerated dose (MTD) was determined to be 0.8 mg/kg. Additional patients were enrolled into the study in 3 expansion cohorts treated at the MTD to assess safety, pharmacokinetics, biomarkers, and clinical benefit in selected patient populations, including solid tumors (n=25), non-Hodgkin lymphoma (NHL; n=9), and diabetic solid tumor patients (n=6; treated at 0.4 mg/kg). Clinical benefit (patients experiencing complete response [CR], partial response [PR], or stable disease [SD]) was observed in 4 of 5 (80%) endometrial cancer patients treated in this study (Table 7), including one patient with CR and 2 with extended SD lasting more than 8 cycles (more than 224 days).

PIK3CA, BRAF, and KRAS mutations were tested using digital PCR on archival tumor samples and cell free DNA isolated from plasma. Next generation sequencing (NGS) of a panel of tumor genes and immunohistochemistry (IHC) for PTEN protein were also performed on archival tumor samples. Of note, the sole patient in the study with a CR had an endometrial cancer with PTEN loss by IHC and mutations in both the PTEN and PIK3CA genes (Table 7). Of the 2 endometrial cancer patients with extended SD lasting more than 8 cycles, PTEN data could only be generated for 1 (patient #2117), and this tumor was also PTEN-negative by IHC (Table 7). The other endometrial cancer patient with extended SD (with 24.3% tumor shrinkage) harbored a KRAS tumor mutation (Table 7). This data shows that Compound A (copanlisib) may provide clinical benefit to patients with endometrial cancer either with or without PIK3CA mutations, with or without PTEN loss or mutation, and with or without mutations in KRAS. Activation of PI3K pathway signaling via a number of mechanisms alone or in combination, such as PTEN loss and/or mutation, PIK3CA mutation, and/or KRAS mutation, may enrich Compound A (copanlisib) activity in this population.

TABLE 7
Clinical outcomes and biomarker data among endometrial cancer patients treated in a phase I study of Compound A (copanlisib)

| | Nb Prior | | | | Best % | | | PTEN | | |
|---|---|---|---|---|---|---|---|---|---|---|
| | Systemic | Stage | | | change in | | | protein | | |
| | Anti-cancer | At | Compound A | | tumor size | PIK3CA | PTEN | (IHC, % | BRAF | KRAS |
| | Therapy | Study | (copanlisib) | Best | from | mutation | mutation | of cells | mutation | mutation |
| Pt # | (setting) | Entry | dose | response | screening | status | status | positive) | status | status |
| 2004 | 2 | IV | 0.4 mg/kg | SD 8 | −24.3 | WT | Nd | nd | WT | MUT |
| | (adjuvant) | | (40 mg) | cycles | | | | | | |
| 2008 | 2 | IV | 0.8 mg/kg | PD | 2.4 | MUT | Nd | nd | WT | WT |
| | (adjuvant) | | (40 mg) | | | (H1047R) | | | | |
| 2116 | 2 | IV | 0.8 mg/kg | SD 2 | −8.7 | WT | Nd | 25% | WT | WT |
| | (palliative) | | (59 mg) | cycles | | | | (PTEN- | | |
| | | | | | | | | positive) | | |
| 2117 | 2 | IIIC | 0.8 mg/kg | SD 8 | −9.1 | WT | Nd | 0% | WT | WT |
| | (adjuvant) | | (51 mg) | cycles | | | | (PTEN- | | |
| | | | | | | | | negative) | | |
| 3106 | 0* | IV | 0.8 mg/kg | CR^{#} | −61.8 | MUT | MUT | 0% | WT | WT |
| | (adjuvant) | | (46 mg, | | | (T1052K/R88L) | | (PTEN- | | |
| | | | then 37 mg°) | | | | | negative) | | |

Pt, patient;
WT, wild-type;
MUT, mutant;
nd, not done
SD, Stable disease;
CR, Complete response;
PR, Partial response
*Only surgery, patient declined adjuvant chemotherapy and radiation
^{#}PR at end of cycle 2 until end of cycle 8, then CR until end of cycle 14
°Dose reduction at cycle 5 due to adverse event

These findings provide a rationale to develop personalized therapies for the treatment of endometrial cancer (hereinafter abbreviated to “EC”), particularly 1st line, 2nd line, relapsed, refractory, type I or type II EC, or endometriosis.

Hence, as mentioned supra, the present invention relates to the use of biomarkers which is the loss of tumor suppressor PTEN or FBXW7, either alone or in combination with another form of PI3K pathway activation (as described in the next paragraph), for predicting the sensitivity and/or resistance of a patient with endometrial cancer (hereinafter abbreviated to “EC”), particularly 1st line, 2nd line, relapsed, refractory, type I or type II EC, or endometriosis, to a 2,3-dihydroimidazo[1,2-c]quinazoline compound as defined herein, thus providing rationale-based dosage as defined herein to overcome resistance (patient selection or stratification). Other forms of PI3K pathway activation include, but are not limited to, perturbation of any of the following alone or in combination: mutation in PIK3CA, PIK3CB, PIK3CD, PIK3CG, PIK3R1, PIK3R2, PIK3R3, PIK3R4, PIK3R5, FGFR1, FGFR2, FGFR3 and/or FGFR4. PTEN loss and alteration of PIK3CA, PIK3CB, PIK3CD, PIK3CG, PIK3R1, PIK3R2, PIK3R3, PIK3R4, PIK3R5, FGFR1, FGFR2, FGFR3 and/or FGFR4 may be measured at either the protein level, mRNA level, or DNA level.

In accordance with an embodiment, the present invention relates to a method of determining the loss of tumor suppressor PTEN or FBXW7.

In accordance with another embodiment, the present invention relates to a method for determining perturbations in PIK3CA, PIK3CB, PIK3CD, PIK3CG, PIK3R1, PIK3R2, PIK3R3, PIK3R4, PIK3R5, FGFR1, FGFR2, FGFR3 and/or FGFR4. PTEN loss and alteration of PIK3CA, PIK3CB, PIK3CD, PIK3CG, PIK3R1, PIK3R2, PIK3R3, PIK3R4, PIK3R5, FGFR1, FGFR2, FGFR3 and/or FGFR4.

Further, as mentioned supra, the present invention thus relates to combinations of:

a) a 2,3-dihydroimidazo[1,2-c]quinazoline compound as defined supra, or a physiologically acceptable salt, solvate, hydrate or stereoisomer thereof; or pharmaceutical compositions containing such a compound or a physiologically acceptable salt, solvate, hydrate or stereoisomer thereof;

and

b) one or more further active agents, in particular an active agent selected from an anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agent as defined supra.

In accordance a particular embodiment of any of the above aspects, or embodiments thereof, of the present invention, said cancer is endometrial cancer (hereinafter abbreviated to “EC”), particularly 1st line, 2nd line, relapsed, refractory, type I or type II EC, or endometriosis.

### REFERENCES

1. Abbosh, P. H.; Nephew, K. P. Multiple signaling pathways converge on b-catenin in thyroid cancer. Thyroid 2005, 15, 551-561.

2. Ali, I. U.; Schriml, L. M.; Dean, M. Mutational spectra of PTEN/MMAC1 gene: a tumor suppressor with lipid phosphatase activity. J. Natl. Cancer Inst. 1999, 91, 1922-1932.

3. Bachman, K. E.; Argani, P.; Samuels, Y.; Silliman, N.; Ptak, J.; Szabo, S.; Konishi, H.; Karakas, B.; Blair, B. G.; Lin, C.; Peters, B. A.; Velculescu, V. E.; Park, B. H. The PIK3CA gene is mutated with high frequency in human breast cancers. Cancer Biol. Therap. 2004, 3, 772-775.

4. Bader, A. G.; Kang, S.; Vogt, P. K. Cancer-specific mutations in PIK3CA are oncogenic in vivo. Proc. Natl. Acad. Sci. U.S.A. 2006, 103, 1475-1479.

5. Barthwal, M. K.; Sathyanarayana, P.; Kundu, C. N.; Rana, B.; Pradeep, A.; Sharma, C.; Woodgett, J. R.; Rana, A. Negative Regulation of Mixed Lineage Kinase 3 by Protein Kinase B/AKT Leads to Cell Survival. J. Biol. Chem. 2003, 278, 3897-3902.

6. Bénistant, C.; Chapuis, H.; Roche, S. A specific function for phosphatidylinositol 3-kinase a (p85a-p110a) in cell survival and for phosphatidylinositol 3-kinase b (p85a-p110b) in de novo DNA synthesis of human colon carcinoma cells. Oncogene 2000, 19, 5083-5090.

7. Broderick, D. K.; Di, C.; Parrett, T. J.; Samuels, Y. R.; Cummins, J. M.; McLendon, R. E.; Fults, D. W.; Velculescu, V. E.; Bigner, D. D.; Yan, H. Mutations of PIK3CA in anaplastic oligodendrogliomas, high-grade astrocytomas, and medulloblastomas. Cancer Res. 2004, 64, 5048-5050.

8. Brown, R. A.; Shepherd, P. R. Growth factor regulation of the novel class II phosphoinositide 3-kinases. Biochem. Soc. Trans. 2001, 29, 535-537.

9. Brunet, A.; Bonni, A.; Zigmond, M. J.; Lin, M. Z.; Juo, P.; Hu, L. S.; Anderson, M. J.; Arden, K. C.; Blenis, J.; Greenberg, M. E. Akt promotes cell survival by phosphorylating and inhibiting a Forkhead transcription factor. Cell 1999, 96, 857-868.

10. Byun, D.-S.; Cho, K.; Ryu, B.-K.; Lee, M.-G.; Park, J.-I.; Chae, K.-S.; Kim, H.-J.; Chi, S.-G. Frequent monoallelic deletion of PTEN and its reciprocal association with PIK3CA amplification in gastric carcinoma. Int. J. Cancer 2003, 104, 318-327.

11. Campbell, I. G.; Russell, S. E.; Choong, D. Y. H.; Montgomery, K. G.; Ciavarella, M. L.; Hooi, C. S. F.; Cristiano, B. E.; Pearson, R. B.; Phillips, W. A. Mutation of the PIK3CA gene in ovarian and breast cancer. Cancer Res. 2004, 64, 7678-7681.

12. Cardone, M. H.; Roy, N.; Stennicke, H. R.; Salvesen, G. S.; Franke, T. F.; Stanbridge, E.; Frisch, S.; Reed, J. C. Regulation of cell death protease caspase-9 by phosphorylation. Science 1998, 282, 1318-1321.

13. Chen, Y. L.; Law, P.-Y.; Loh, H. H. Inhibition of PI3K/Akt signaling: An emerging paradigm for targeted cancer therapy. Curr. Med. Chem. Anticancer Agents 2005, 5, 575-589.

14. Ciechomska, I.; Pyrzynska, B.; Kazmierczak, P.; Kaminska, B. Inhibition of Akt kinase signaling and activation of Forkhead are indispensable for up-regulation of FasL expression in apoptosis of glioma cells. Oncogene 2003, 22, 7617-7627.

15. Cross, D. A. E.; Alessi, D. R.; Cohen, P.; Andjelkovich, M.; Hemmings, B. A. Inhibition of glycogen synthase kinase-3 by insulin mediated by protein kinase B. Nature 1995, 378, 785-9.

16. Cully, M.; You, H.; Levine, A. J.; Mak, T. W. Beyond PTEN mutations: the PI3K pathway as an integrator of multiple inputs during tumorigenesis. Nat. Rev. Cancer 2006, 6, 184-192.

17. Czauderna, F.; Fechtner, M.; Aygun, H.; Arnold, W.; Klippel, A.; Giese, K.; Kaufmann, J. Functional studies of the PI(3)-kinase signaling pathway employing synthetic and expressed siRNA. Nucleic Acids Res. 2003, 31, 670-682.

18. del Peso, L.; González-Garcia, M.; Page, C.; Herrera, R.; Nunez, G. Interleukin-3-induced phosphorylation of BAD through the protein kinase Akt. Science 1997, 278, 687-689.

19. Diehl, J. A.; Cheng, M.; Roussel, M. F.; Sherr, C. J. Glycogen synthase kinase-3b regulates cyclin D1 proteolysis and subcellular localization. Genes Dev. 1998, 12, 3499-3511.

20. Dijkers, P. F.; Medema, R. H.; Lammers, J.-W. J.; Koenderman, L.; Coffer, P. J. Expression of the pro-apoptotic Bcl-2 family member Bim is regulated by the Forkhead transcription factor FKHR-L1. Curr. Biol. 2000, 10, 1201-1204.

21. Domin, J.; Waterfield, M. D. Using structure to define the function of phosphoinositide 3-kinase family members. FEBS Lett. 1997, 410, 91-95.

22. Downes, C. P.; Gray, A.; Lucocq, J. M. Probing phosphoinositide functions in signaling and membrane trafficking. Trends Cell Biol. 2005, 15, 259-268.

23. Figueroa, C.; Tarras, S.; Taylor, J.; Vojtek, A. B. Akt2 negatively regulates assembly of the POSH-MLK-JNK signaling complex. J. Biol. Chem. 2003, 278, 47922-47927.

24. Fleming, I. N.; Gray, A.; Downes, C. P. Regulation of the Rac1-specific exchange factor tiam1 involves both phosphoinositide 3-kinase-dependent and -independent components. Biochem. J. 2000, 351, 173-182.

25. Funaki, M.; Katagiri, H.; Inukai, K.; Kikuchi, M.; Asano, T. Structure and function of phosphatidylinositol-3,4 kinase. Cell. Signal. 2000, 12, 135-142.

26. Gallia, G. L.; Rand, V.; Siu, I. M.; Eberhart, C. G.; James, C. D.; Marie, S. K. N.; Oba-Shinjo, S. M.; Carlotti, C. G.; Caballero, O. L.; Simpson, A. J. G.; Brock, M. V.; Massion, P. P.; Carson, B. S., Sr.; Riggins, G. J. PIK3CA gene mutations in pediatric and adult glioblastoma multiforme. Mol. Cancer Res. 2006, 4, 709-714.

27. Gershtein, E. S.; Shatskaya, V. A.; Ermilova, V. D.; Kushlinsky, N. E.; Krasil'nikov, M. A. Phosphatidylinositol 3-kinase expression in human breast cancer. Clin. Chim. Acta 1999, 287, 59-67.

28. Gottschalk, A. R.; Doan, A.; Nakamura, J. L.; Stokoe, D.; Haas-Kogan, D. A. Inhibition of phosphatidylinositol-3-kinase causes increased sensitivity to radiation through a PKB-dependent mechanism. Int. J. Radiat. Oncol. Biol. Phys. 2005, 63, 1221-1227.

29. Gupta, A. K.; Cerniglia, G. J.; Mick, R.; Ahmed, M. S.; Bakanauskas, V. J.; Muschel, R. J.; McKenna, W. G. Radiation sensitization of human cancer cells in vivo by inhibiting the activity of PI3K using LY294002. Int. J. Radiat. Oncol. Biol. Phys. 2003, 56, 846-853.

30. Haas-Kogan, D.; Shalev, N.; Wong, M.; Mills, G.; Yount, G.; Stokoe, D. Protein kinase B (PKB/Akt) activity is elevated in glioblastoma cells due to mutation of the tumor suppressor PTEN/MMAC. Curr. Biol. 1998, 8, 1195-1198.

31. Hartmann, C.; Bartels, G.; Gehlhaar, C.; Holtkamp, N.; von Deimling, A. PIK3CA mutations in glioblastoma multiforme. Acta Neuropathol. 2005, 109, 639-642.

32. Hennessy, B. T.; Smith, D. L.; Ram, P. T.; Lu, Y.; Mills, G. B. Exploiting the PI3K/AKT Pathway for Cancer Drug Discovery. Nat. Rev. Drug Disc. 2005, 4, 988-1004.

33. Hodgkinson, C. P.; Sale, E. M.; Sale, G. J. Characterization of PDK2 activity against Protein Kinase B gamma. Biochemistry 2002, 41, 10351-10359.

34. Hresko, R. C.; Murata, H.; Mueckler, M. Phosphoinositide-dependent Kinase-2 is a distinct protein kinase enriched in a novel cytoskeletal fraction associated with adipocyte plasma membranes. J. Biol. Chem. 2003, 278, 21615-21622.

35. Huang, C.; Ma, W.-Y.; Dong, Z. Requirement for phosphatidylinositol 3-kinase in epidermal growth factor-induced AP-1 transactivation and transformation in JB6 P+ cells. Mol. Cell. Biol. 1996, 16, 6427-6435.

36. Hupp, T. R.; Lane, D. P.; Ball, K. L. Strategies for manipulating the p53 pathway in the treatment of human cancer. Biochem. J. 2000, 352, 1-17.

37. Ihle, N. T.; Williams, R.; Chow, S.; Chew, W.; Berggren, M. I.; Paine-Murrieta, G.; Minion, D. J.; Halter, R. J.; Wipf, P.; Abraham, R.; Kirkpatrick, L.; Powis, G. Molecular pharmacology and antitumor activity of PX-866, a novel inhibitor of phosphoinositide-3-kinase signaling. Mol. Cancer Therap. 2004, 3, 763-772.

38. Ikenoue, T.; Kanai, F.; Hikiba, Y.; Obata, T.; Tanaka, Y.; Imamura, J.; Ohta, M.; Jazag, A.; Guleng, B.; Tateishi, K.; Asaoka, Y.; Matsumura, M.; Kawabe, T.; Omata, M. Functional analysis of PIK3CA gene mutations in human colorectal cancer. Cancer Res. 2005, 65, 4562-4567.

39. Ishii, N.; Maier, D.; Merlo, A.; Tada, M.; Sawamura, Y.; Diserens, A.-C.; Van Meir, E. G. Frequent co-alterations of TP53, p16/CDKN2A, p14ARF, PTEN tumor suppressor genes in human glioma cell lines. Brain Pathol. 1999, 9, 469-479.

40. Itoh, T.; Takenawa, T. Phosphoinositide-binding domains. Functional units for temporal and spatial regulation of intracellular signaling. Cell. Signal. 2002, 14, 733-743.

41. Janssen, J. W. G.; Schleithoff, L.; Bartram, C. R.; Schulz, A. S. An oncogenic fusion product of the phosphatidylinositol 3-kinase p85b subunit and HUMORF8, a putative deubiquitinating enzyme. Oncogene 1998, 16, 1767-1772.

42. Jimenez, C.; Jones, D. R.; Rodriguez-Viciana, P.; Gonzalez-Garcia, A.; Leonardo, E.; Wennstrom, S.; Von Kobbe, C.; Toran, J. L.; R.-Borlado, L.; Calvo, V.; Copin, S. G.; Albar, J. P.; Gaspar, M. L.; Diez, E.; Marcos, M. A. R.; Downward, J.; Martinez-A, C.; Merida, I.; Carrera, A. C. Identification and characterization of a new oncogene derived from the regulatory subunit of phosphoinositide 3-kinase. EMBO J. 1998, 17, 743-753.

43. Jucker, M.; Sudel, K.; Horn, S.; Sickel, M.; Wegner, W.; Fiedler, W.; Feldman, R. A. Expression of a mutated form of the p85a regulatory subunit of phosphatidylinositol 3-kinase in a Hodgkin's lymphoma-derived cell line (CO). Leukemia 2002, 16, 894-901.

44. Kang, S.; Bader, A. G.; Vogt, P. K. Phosphatidylinositol 3-kinase mutations identified in human cancer are oncogenic. Proc. Natl. Acad. Sci. U.S.A. 2005, 102, 802-807.

45. Kang, S.; Denley, A.; Vanhaesebroeck, B.; Vogt, P. K. Oncogenic transformation induced by the p110b, -g, and -d isoforms of class I phosphoinositide 3-kinase. Proc. Natl. Acad. Sci. U.S.A. 2006, 103, 1289-1294.

46. Katso, R.; Okkenhaug, K.; Ahmadi, K.; White, S.; Timms, J.; Waterfield, M. D. Cellular function of phosphoinositide 3-kinases: implications for development, immunity, homeostasis, and cancer. Annu. Rev. Cell Dev. Biol. 2001, 17, 615-675.

47. Kim, A. H.; Khursigara, G.; Sun, X.; Franke, T. F.; Chao, M. V. Akt phosphorylates and negatively regulates apoptosis signal-regulating kinase 1. Mol. Cell. Biol. 2001, 21, 893-901.

48. Kim, D.; Dan, H. C.; Park, S.; Yang, L.; Liu, Q.; Kaneko, S.; Ning, J.; He, L.; Yang, H.; Sun, M.; Nicosia, S. V.; Cheng, J. Q. AKT/PKB signaling mechanisms in cancer and chemoresistance. Front. Biosci. 2005, 10, 975-987.

49. Klippel, A.; Kavanaugh, W. M.; Pot, D.; Williams, L. T. A specific product of phosphatidylinositol 3-kinase directly activates the protein kinase Akt through its pleckstrin homology domain. Mol. Cell. Biol. 1997, 17, 338-44.

50. Kodaki, T.; Woscholski, R.; Hallberg, B.; Rodriguez-Viciana, P.; Downward, J.; Parker, P. J. The activation of phosphatidylinositol 3-kinase by Ras. Curr. Biol. 1994, 4, 798-806.

51. Kops, G. J. P. L.; De Ruiter, N. D.; De Vries-Smits, A. M. M.; Powell, D. R.; Bos, J. L.; Burgering, B. M. T. Direct control of the Forkhead transcription factor AFX by protein kinase B. Nature 1999, 398, 630-634.

52. Lee, J. T., Jr.; Steelman, L. S.; McCubrey, J. A. Phosphatidylinositol 3′-Kinase Activation Leads to Multidrug Resistance Protein-1 Expression and Subsequent Chemoresistance in Advanced Prostate Cancer Cells. Cancer Res. 2004, 64, 8397-8404.

53. Lee, J. W.; Soung, Y. H.; Kim, S. Y.; Lee, H. W.; Park, W. S.; Nam, S. W.; Kim, S. H.; Lee, J. Y.; Yoo, N. J.; Lee, S. H. PIK3CA gene is frequently mutated in breast carcinomas and hepatocellular carcinomas. Oncogene 2005, 24, 1477-1480.

54. Lemmon, M. A. Phosphoinositide recognition domains. Traffic 2003, 4, 201-213.

55. Levine, D. A.; Bogomolniy, F.; Yee, C. J.; Lash, A.; Barakat, R. R.; Borgen, P. I.; Boyd, J. Frequent Mutation of the PIK3CA Gene in Ovarian and Breast Cancers. Clin. Cancer Res. 2005, 11, 2875-2878.

56. Li, J.; Yen, C.; Liaw, D.; Podsypanina, K.; Bose, S.; Wang, S. I.; Puc, J.; Miliaresis, C.; Rodgers, L.; McCombie, R.; Bigner, S. H.; Giovanella, B. C.; Ittmann, M.; Tycko, B.; Hibshoosh, H.; Wigler, M. H.; Parsons, R. PTEN, a putative protein tyrosine phosphatase gene mutated in human brain, breast, and prostate cancer. Science 1997, 275, 1943-1947.

57. Li, V. S. W.; Wong, C. W.; Chan, T. L.; Chan, A. S. W.; Zhao, W.; Chu, K.-M.; So, S.; Chen, X.; Yuen, S. T.; Leung, S. Y. Mutations of PIK3CA in gastric adenocarcinoma. BMC Cancer 2005, 5, 29.

58. Liao, Y.; Hung, M.-C. Regulation of the activity of p38 mitogen-activated protein kinase by Akt in cancer and adenoviral protein E1A-mediated sensitization to apoptosis. Mol. Cell. Biol. 2003, 23, 6836-6848.

59. Lopez-Ilasaca, M.; Li, W.; Uren, A.; Yu, J.-c.; Kazlauskas, A.; Gutkind, J. S.; Heidaran, M. A. Requirement of phosphatidylinositol-3 kinase for activation of JNK/SAPKs by PDGF. Biochem. Biophys. Res. Commun. 1997, 232, 273-277.

60. Ma, Y.-Y.; Wei, S.-J.; Lin, Y.-C.; Lung, J.-C.; Chang, T.-C.; Whang-Peng, J.; Liu, J. M.; Yang, D.-M.; Yang, W. K.; Shen, C.-Y. PIK3CA as an oncogene in cervical cancer. Oncogene 2000, 19, 2739-2744.

61. Mayo, L. D.; Dixon, J. E.; Durden, D. L.; Tonks, N. K.; Donner, D. B. PTEN protects p53 from Mdm2 and sensitizes cancer cells to chemotherapy. J. Biol. Chem. 2002, 277, 5484-5489.

62. Momand, J.; Wu, H.-H.; Dasgupta, G. MDM2 - master regulator of the p53 tumor suppressor protein. Gene 2000, 242, 15-29.

63. Motti, M. L.; De Marco, C.; Califano, D.; Fusco, A.; Viglietto, G. Akt-dependent T198 phosphorylation of cyclin-dependent kinase inhibitor p27kip1 in breast cancer. Cell Cycle 2004, 3, 1074-1080.

64. Myers, M. P.; Pass, I.; Batty, I. H.; Van Der Kaay, J.; Stolarov, J. P.; Hemmings, B. A.; Wigler, M. H.; Downes, C. P.; Tonks, N. K. The lipid phosphatase activity of PTEN is critical for its tumor suppressor function. Proc. Natl. Acad. Sci. U.S.A. 1998, 95, 13513-13518.

65. Nagata, Y.; Lan, K.-H.; Zhou, X.; Tan, M.; Esteva, F. J.; Sahin, A. A.; Klos, K. S.; Li, P.; Monia, B. P.; Nguyen, N. T.; Hortobagyi, G. N.; Hung, M.-C.; Yu, D. PTEN activation contributes to tumor inhibition by trastuzumab, and loss of PTEN predicts trastuzumab resistance in patients. Cancer Cell 2004, 6, 117-127.

66. Naito, A. T.; Akazawa, H.; Takano, H.; Minamino, T.; Nagai, T.; Aburatani, H.; Komuro, I. Phosphatidylinositol 3-Kinase-Akt Pathway Plays a Critical Role in Early Cardiomyogenesis by Regulating Canonical Wnt Signaling. Circ. Res. 2005, 97, 144-151.

67. Oda, K.; Stokoe, D.; Taketani, Y.; McCormick, F. High Frequency of Coexistent Mutations of PIK3CA and PTEN Genes in Endometrial Carcinoma. Cancer Res. 2005, 65, 10669-10673.

68. Ogawara, Y.; Kishishita, S.; Obata, T.; Isazawa, Y.; Suzuki, T.; Tanaka, K.; Masuyama, N.; Gotoh, Y. Akt enhances Mdm2-mediated ubiquitination and degradation of p53. J. Biol. Chem. 2002, 277, 21843-21850.

69. Olson, J. M.; Hallahan, A. R. p38 MAP kinase: a convergence point in cancer therapy. Trends Mol. Med. 2004, 10, 125-129.

70. Osaki, M.; Oshimura, M.; Ito, H. PI3K-Akt pathway: Its functions and alterations in human cancer. Apoptosis 2004, 9, 667-676.

71. Pastorino, J. G.; Tafani, M.; Farber, J. L. Tumor necrosis factor induces phosphorylation and translocation of BAD through a phosphatidylinositide-3-OH kinase-dependent pathway. J. Biol. Chem. 1999, 274, 19411-19416.

72. Pendaries, C.; Tronchere, H.; Plantavid, M.; Payrastre, B. Phosphoinositide signaling disorders in human diseases. FEBS Lett. 2003, 546, 25-31.

73. Phillips, W. A.; St. Clair, F.; Munday, A. D.; Thomas, R. J. S.; Mitchell, C. A. Increased levels of phosphatidylinositol 3-kinase activity in colorectal tumors. Cancer 1998, 83, 41-47.

74. Philp, A. J.; Campbell, I. G.; Leet, C.; Vincan, E.; Rockman, S. P.; Whitehead, R. H.; Thomas, R. J. S.; Phillips, W. A. The phosphatidylinositol 3′-kinase p85a gene is an oncogene in human ovarian and colon tumors. Cancer Res. 2001, 61, 7426-7429.

75. Powis, G.; Bonjouklian, R.; Berggren, M. M.; Gallegos, A.; Abraham, R.; Ashendel, C.; Zalkow, L.; Matter, W. F.; Dodge, J. Wortmannin, a potent and selective inhibitor of phosphatidylinositol-3-kinase. Cancer Res. 1994, 54, 2419-23.

76. Pu, P.; Kang, C.; Zhang, Z.; Liu, X.; Jiang, H. Downregulation of PIK3CB by siRNA suppresses malignant glioma cell growth in vitro and in vivo. Technol. Cancer Res. Treat. 2006, 5, 271-280.

77. Rahimi, N.; Tremblay, E.; Elliott, B. Phosphatidylinositol 3-kinase activity is required for hepatocyte growth factor-induced mitogenic signals in epithelial cells. J. Biol. Chem. 1996, 271, 24850-24855.

78. Roche, S.; Downward, J.; Raynal, P.; Courtneidge, S. A. A function for phosphatidylinositol 3-kinase b (p85a-p110b) in fibroblasts during mitogenesis: requirement for insulin- and lysophosphatidic acid-mediated signal transduction. Mol. Cell. Biol. 1998, 18, 7119-7129.

79. Roche, S.; Koegl, M.; Courtneidge, S. A. The phosphatidylinositol 3-kinase a is required for DNA synthesis induced by some, but not all, growth factors. Proc. Natl. Acad. Sci. U.S.A. 1994, 91, 9185-9.

80. Romashkova, J. A.; Makarov, S. S. Nf-kB is a target of Akt in anti-apoptotic PDGF signaling. Nature 1999, 401, 86-90.

81. Saal, L. H.; Holm, K.; Maurer, M.; Memeo, L.; Su, T.; Wang, X.; Yu, J. S.; Malmstroem, P.-O.; Mansukhani, M.; Enoksson, J.; Hibshoosh, H.; Borg, A.; Parsons, R. PIK3CA mutations correlate with hormone receptors, node metastasis, and ERBB2, and are mutually exclusive with PTEN loss in human breast carcinoma. Cancer Res. 2005, 65, 2554-2559.

82. Samuels, Y.; Diaz, L. A., Jr.; Schmidt-Kittler, O.; Cummins, J. M.; DeLong, L.; Cheong, I.; Rago, C.; Huso, D. L.; Lengauer, C.; Kinzler, K. W.; Vogelstein, B.; Velculescu, V. E. Mutant PIK3CA promotes cell growth and invasion of human cancer cells. Cancer Cell 2005, 7, 561-573.

83. Samuels, Y.; Ericson, K. Oncogenic PI3K and its role in cancer. Curr. Opin. Oncol. 2006, 18, 77-82.

84. Samuels, Y.; Wang, Z.; Bardelli, A.; Silliman, N.; Ptak, J.; Szabo, S.; Yan, H.; Gazdar, A.; Powell, S. M.; Riggins, G. J.; Willson, J. K. V.; Markowitz, S.; Kinzler, K. W.; Vogelstein, B.; Velculescu, V. E. Brevia: High frequency of mutations of the PIK3Ca gene in human cancers. Science 2004, 304, 554.

85. Scheid, M. P.; Marignani, P. A.; Woodgett, J. R. Multiple phosphoinositide 3-kinase-dependent steps in activation of protein kinase B. Mol. Cell. Biol. 2002, 22, 6247-6260.

86. Schultz, R. M.; Merriman, R. L.; Andis, S. L.; Bonjouklian, R.; Grindey, G. B.; Rutherford, P. G.; Gallegos, A.; Massey, K.; Powis, G. In vitro and in vivo antitumor activity of the phosphatidylinositol-3-kinase inhibitor, wortmannin. Anticancer Res. 1995, 15, 1135-9.

87. Segrelles, C.; Moral, M.; Lara, M. F.; Ruiz, S.; Santos, M.; Leis, H.; Garcia-Escudero, R.; Martinez-Cruz, A. B.; Martinez-Palacio, J.; Hernandez, P.; Ballestin, C.; Paramio, J. M. Molecular determinants of Akt-induced keratinocyte transformation. Oncogene 2006, 25, 1174-1185.

88. Sekimoto, T.; Fukumoto, M.; Yoneda, Y. 14-3-3 suppresses the nuclear localization of threonine 157-phosphorylated p27Kip1. EMBO J. 2004, 23, 1934-1942.

89. Semba, S.; Itoh, N.; Ito, M.; Youssef, E. M.; Harada, M.; Moriya, T.; Kimura, W.; Yamakawa, M. Down-regulation of PIK3CG catalytic subunit of phosphatidylinositol 3-OH kinase by CpG hypermethylation in human colorectal carcinoma. Clin. Cancer Res. 2002, 8, 3824-3831.

90. Shayesteh, L.; Lu, Y.; Kuo, W.-L.; Baldocchi, R.; Godfrey, T.; Collins, C.; Pinkel, D.; Powell, B.; Mills, G. B.; Gray, J. W. PIK3CA is implicated as an oncogene in ovarian cancer. Nat. Genet. 1999, 21, 99-102.

91. Shekar, S. C.; Wu, H.; Fu, Z.; Yip, S.-C.; Nagajyothi; Cahill, S. M.; Girvin, M. E.; Backer, J. M. Mechanism of Constitutive Phosphoinositide 3-Kinase Activation by Oncogenic Mutants of the p85 Regulatory Subunit. J. Biol. Chem. 2005, 280, 27850-27855.

92. Stahl, J. M.; Cheung, M.; Sharma, A.; Trivedi, N. R.; Shanmugam, S.; Robertson, G. P. Loss of PTEN Promotes Tumor Development in Malignant Melanoma. Cancer Res. 2003, 63, 2881-2890.

93. Stambolic, V.; Suzuki, A.; De La Pompa, J. L.; Brothers, G. M.; Mirtsos, C.; Sasaki, T.; Ruland, J.; Penninger, J. M.; Siderovski, D. P.; Mak, T. W. Negative regulation of PKB/Akt-Dependent cell survival by the tumor suppressor PTEN. Cell 1998, 95, 29-39.

94. Stauffer, F.; Holzer, P.; Garcia-Echeverria, C. Blocking the PI3K/PKB pathway in tumor cells. Curr. Med. Chem. Anticancer Agents 2005, 5, 449-462.

95. Steck, P. A.; Pershouse, M. A.; Jasser, S. A.; Yung, W. K. A.; Lin, H.; Ligon, A. H.; Langford, L. A.; Baumgard, M. L.; Hattier, T.; Davis, T.; Frye, C.; Hu, R.; Swedlund, B.; Teng, D. H. F.; Tavtigian, S. V. Identification of a candidate tumor suppressor gene, MMAC1, at chromosome 10q23.3 that is mutated in multiple advanced cancers. Nat. Genet. 1997, 15, 356-362.

96. Stein, R. C.; Waterfield, M. D. PI3-kinase inhibition: a target for drug development? Mol. Med. Today 2000, 6, 347-358.

97. Stephens, L.; Williams, R.; Hawkins, P. Phosphoinositide 3-kinases as drug targets in cancer. Curr. Opin. Pharmacol. 2005, 5, 357-365.

98. Su, J. D.; Mayo, L. D.; Donner, D. B.; Durden, D. L. PTEN and Phosphatidylinositol 3′-Kinase Inhibitors Up-Regulate p53 and Block Tumor-induced Angiogenesis: Evidence for an Effect on the Tumor and Endothelial Compartment. Cancer Res. 2003, 63, 3585-3592.

99. Tanaka, M.; Grossman, H. B. In vivo gene therapy of human bladder cancer with PTEN suppresses tumor growth, downregulates phosphorylated Akt, and increases sensitivity to doxorubicin. Gene Ther. 2003, 10, 1636-1642.

100. Tang, E. D.; Nunez, G.; Barr, F. G.; Guan, K.-L. Negative regulation of the forkhead transcription factor FKHR by Akt. J. Biol. Chem. 1999, 274, 16741-16746.

101. Taylor, V.; Wong, M.; Brandts, C.; Reilly, L.; Dean, N. M.; Cowsert, L. M.; Moodie, S.; Stokoe, D. 5′ Phospholipid phosphatase SHIP-2 causes protein kinase B inactivation and cell cycle arrest in glioblastoma cells. Mol. Cell. Biol. 2000, 20, 6860-6871.

102. Toker, A. Phosphoinositides and signal transduction. Cell. Mol. Life Sci. 2002, 59, 761-779.

103. Traer, C. J.; Foster, F. M.; Abraham, S. M.; Fry, M. J. Are class II phosphoinositide 3-kinases potential targets for anticancer therapies? Bull. Cancer (Paris). 2006, 93, E53-8.

104. Vanhaesebroeck, B.; Leevers, S. J.; Ahmadi, K.; Timms, J.; Katso, R.; Driscoll, P. C.; Woscholski, R.; Parker, P. J.; Waterfield, M. D. Synthesis and function of 3-phosphorylated inositol lipids. Annu. Rev. Biochem. 2001, 70, 535-602.

105. Vanhaesebroeck, B.; Waterfield, M. D. Signaling by Distinct Classes of Phosphoinositide 3-Kinases. Exp. Cell Res. 1999, 253, 239-254.

106. Vivanco, I.; Sawyers, C. L. The phosphatidylinositol 3-Kinase-AKT pathway in human cancer. Nat. Rev. Cancer 2002, 2, 489-501.

107. Wang, Y.; Helland, A.; Holm, R.; Kristensen Gunnar, B.; Borresen-Dale, A.-L. PIK3CA mutations in advanced ovarian carcinomas. Hum. Mutat. 2005, 25, 322.

108. West, K. A.; Castillo, S. S.; Dennis, P. A. Activation of the PI3K/Akt pathway and chemotherapeutic resistance. Drug Resist. Update. 2002, 5, 234-48.

109. Whyte, D. B.; Holbeck, S. L. Correlation of PIK3Ca mutations with gene expression and drug sensitivity in NCI-60 cell lines. Biochem. Biophys. Res. Commun. 2006, 340, 469-475.

110. Wilker, E.; Lu, J.; Rho, O.; Carbajal, S.; Beltran, L.; DiGiovanni, J. Role of PI3K/Akt signaling in insulin-like growth factor-1 (IGF-1) skin tumor promotion. Mol. Carcinog. 2005, 44, 137-145.

111. Workman, P. Inhibiting the phosphoinositide 3-kinase pathway for cancer treatment. Biochem. Soc. Trans. 2004, 32, 393-396.

112. Wu, G.; Xing, M.; Mambo, E.; Huang, X.; Liu, J.; Guo, Z.; Chatterjee, A.; Goldenberg, D.; Gollin, S. M.; Sukumar, S.; Trink, B.; Sidransky, D. Somatic mutation and gain of copy number of PIK3CA in human breast cancer. Breast Cancer Res. 2005, 7, R609-R616.

113. Wymann, M. P.; Sozzani, S.; Altruda, F.; Mantovani, A.; Hirsch, E. Lipids on the move: phosphoinositide 3-kinases in leukocyte function. Immunol. Today 2000, 21, 260-264.

114. Yap, D. B.; Hsieh, J. K.; Lu, X. Mdm2 inhibits the apoptotic function of p53 mainly by targeting it for degradation. J. Biol. Chem. 2000, 275, 37296-302.

115. Yuan, Z.-q.; Feldman, R. I.; Sussman, G. E.; Coppola, D.; Nicosia, S. V.; Cheng, J. Q. AKT2 Inhibition of Cisplatin-induced JNK/p38 and Bax Activation by Phosphorylation of ASK1: Implication of AKT2 in Chemoresistance. J. Biol. Chem. 2003, 278, 23432-23440.

116. Zhao, H.; Dupont, J.; Yakar, S.; Karas, M.; LeRoith, D. PTEN inhibits cell proliferation and induces apoptosis by downregulating cell surface IGF-IR expression in prostate cancer cells. Oncogene 2004, 23, 786-794.

117. Zhao, J. J.; Cheng, H.; Jia, S.; Wang, L.; Gjoerup, O. V.; Mikami, A.; Roberts, T. M. The p110a isoform of PI3K is essential for proper growth factor signaling and oncogenic transformation. Proc. Natl. Acad. Sci. U.S.A. 2006, 103, 16296-300.

118. Zhou, B. P.; Liao, Y.; Xia, W.; Spohn, B.; Lee, M.-H.; Hung, M.-C. Cytoplasmic localization of p21Cip1/WAF1 by Akt-induced phosphorylation in HER-2/neu-overexpressing cells. Nat. Cell Biol. 2001, 3, 245-252.

### REFERENCES

1A. Kenkre V P, Kahl B S. Curr Hematol Malig Rep 2012; 7: 216-220

2A. Iyengar S et al. Blood 2013; [Epub ahead of print]

3A. Liu N et al. Poster 4476 presented at the 101st Annual Meeting of the American Association for Cancer Research, Washington D.C., USA, Apr. 17-21, 2010

4A. Ziegelbauer K et al. Br J Pharmacol 2005; 145: 178-192

5A. Puri K D, Gold M R. Front Immunol 2012; 3: 256

6A. Patnaik A et al. Poster 3704 presented at the 54th ASH Annual meeting and exposition, Atlanta, Ga., USA, Dec. 8-11, 2012

7A. Chou T C. Pharmacol Rev 2006; 58: 621-681

1. A method for treatment or prophylaxis of endometrial cancer, comprising administering to a patient in need thereof a therapeutically effective amount of a compound of formula (I): 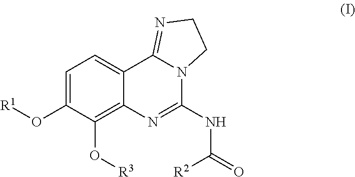
or a physiologically acceptable salt, solvate, hydrate or stereoisomer thereof, in which:
R¹ represents —(CH₂)ₙ—(CHR⁴)—(CH₂)ₘ—N(R⁵)(R⁵);
R² represents a heteroaryl optionally substituted with 1, 2 or 3 R⁶ groups;
R³ represents alkyl or cycloalkyl;
R⁴ represents hydrogen, hydroxy or alkoxy;
R⁵ and R^{5′} may be the same or different and are independently, hydrogen, alkyl, cycloalkylalklyl, or alkoxyalkyl or R⁵ and R^{5′} may be taken together with the nitrogen atom to which they are bound to form a 3-7 membered nitrogen containing heterocyclic ring optionally containing at least one additional heteroatom selected from oxygen, nitrogen or sulfur and which may be optionally substituted with 1 or more R^{6′} groups, or R⁴ and R⁵ may be taken together with the atoms to which they are bound to form a 5-6 membered nitrogen containing heterocyclic ring optionally containing 1 or more nitrogen, oxygen or sulfur atoms and which may be optionally substituted with 1 or more R^{6′} groups;
each occurrence of R⁶ may be the same or different and is independently halogen, alkyl, alkenyl, alkynyl, cycloalkyl, cycloalkylalklyl, aryl, arylalkyl, heteroaryl, heteroarylalkyl, heterocyclic ring, heterocyclylalkyl, alkyl-OR⁷, alkyl-SR⁷, alkyl-N(R⁷)(R^{7′}), alkyl-COR⁷, —CN, —COOR⁷, —CON(R⁷)(R^{7′}), —OR⁷, —SR⁷, —N(R⁷)(R^{7′}), or —NR⁷COR⁷ each of which may be optionally substituted with 1 or more R⁸ groups;
each occurrence of R^{6′} may be the same or different and is independently alkyl, cycloalkylalklyl, or alkyl-OR⁷;
each occurrence of R⁷ and R^{7′} may be the same or different and is independently hydrogen, alkyl, alkenyl, alkynyl, cycloalkyl, cycloalkylalklyl, cycloalkenyl, aryl, arylalkyl, heteroaryl, heterocyclic ring, heterocyclylalkyl, or heteroarylalkyl;
each occurrence of R⁸ is independently nitro, hydroxy, cyano, formyl, acetyl, halogen, amino, alkyl, alkoxy, alkenyl, alkynyl, cycloalkyl, cycloalkylalklyl, cycloalkenyl, aryl, arylalkyl, heteroaryl, heterocyclic ring, heterocyclylalkyl, or heteroarylalkyl; and
n is an integer from 1-4 and m is an integer from 0-4 with the proviso that when when R⁴ and R⁵ are taken together with the atoms to which they are bound to form a 3-7 membered nitrogen containing ring, n+m≦4
or a physiologically acceptable salt, solvate, hydrate or stereoisomer thereof.

2. The method according to claim 1, wherein R⁴ is hydroxy.

3. The method according to claim 1, wherein R⁴ and R⁵ are taken together with the atoms to which they are bound to form a 5-6 membered nitrogen containing heterocyclic ring optionally containing 1 or more nitrogen, oxygen or sulfur atoms and which may be optionally substituted with 1 or more R^{6′} groups.

4. The method according to claim 1, wherein R² is pyridine, pyridazine, pyrimidine, pyrazine, pyrole, oxazole, thiazole, furan or thiophene, optionally substituted with 1, 2 or 3 R⁶ groups.

5. The method according to claim 1, wherein said compound of formula (I) has the formula:

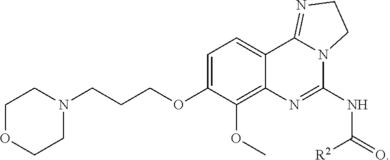

6. The method according to claim 5, wherein R² is pyridine, pyridazine, pyrimidine, pyrazine, pyrole, oxazole, thiazole, furan or thiophene, optionally substituted with 1, 2 or 3 R⁶ groups.

7. The method according to claim 1, wherein said compound is selected from the group consisting of:
N-[7-methoxy-8-(3-morpholin-4-ylpropoxy)-2,3-dihydroimidazo[1,2-c]quinazolin-5-yl]pyrimidine-5-carboxamide;
N-(8-{3-[(2R,6S)-2,6-dimethylmorpholin-4-yl]propoxy}-7-methoxy-2,3-dihydroimidazo[1,2-c]quinazolin-5-yl)nicotinamide;
N-(8-{3-[(2R,6S)-2,6-dimethylmorpholin-4-yl]propoxy}-7-methoxy-2,3-dihydroimidazo[1,2-c]quinazolin-5-yl)-2,4-dimethyl-1,3-thiazole-5-carboxamide;
2-amino-N-[7-methoxy-8-(3-morpholin-4-ylpropoxy)-2,3-dihydroimidazo[1,2-c]quinazolin-5-yl]-1,3-thiazole-5-carboxamide;
2-amino-N-[7-methoxy-8-(3-morpholin-4-ylpropoxy)-2,3-dihydroimidazo[1,2-c]quinazolin-5-yl]isonicotinamide;
2-amino-N-[7-methoxy-8-(3-morpholin-4-ylpropoxy)-2,3-dihydroimidazo[1,2-c]quinazolin-5-yl]-4-methyl-1,3-thiazole-5-carboxamide;
2-amino-N-[7-methoxy-8-(3-morpholin-4-ylpropoxy)-2,3-dihydroimidazo[1,2-c]quinazolin-5-yl]-4-propylpyrimidine-5-carboxamide;
N-{8-[2-(4-ethylmorpholin-2-yl)ethoxy]-7-methoxy-2,3-dihydroimidazo[1,2-c]quinazolin-5-yl}nicotinamide;
N-{8-[2-(dimethylamino)ethoxy]-7-methoxy-2,3-dihydroimidazo[1,2-c]quinazolin-5-yl}pyrimidine-5-carboxamide;
N-(8-{3-[2-(hydroxymethyl)morpholin-4-yl]propoxy}-7-methoxy-2,3-dihydroimidazo[1,2-c]quinazolin-5-yl)nicotinamide;
N-(8-{3-[2-(hydroxymethyl)morpholin-4-yl]propoxy}-7-methoxy-2,3-dihydroimidazo[1,2-c]quinazolin-5-yl)nicotinamide;
N-{8-[3-(dimethylamino)propoxy]-7-methoxy-2,3-dihydroimidazo[1,2-c]quinazolin-5-yl}nicotinamide 1-oxide;
2-amino-N-[7-methoxy-8-(3-morpholin-4-ylpropoxy)-2,3-dihydroimidazo[1,2-c]quinazolin-5-yl]pyrimidine-5-carboxamide;
N-[7-methoxy-8-(3-morpholin-4-ylpropoxy)-2,3-dihydroimidazo[1,2-c]quinazolin-5-yl]-6-(2-pyrrolidin-1-ylethyl)nicotinamide;
6-(cyclopentylamino)-N-[7-methoxy-8-(3-morpholin-4-ylpropoxy)-2,3-dihydroimidazo[1,2-c]quinazolin-5-yl]nicotinamide;
N-[8-(2-hydroxy-3-morpholin-4-ylpropoxy)-7-methoxy-2,3-dihydroimidazo[1,2-c]quinazolin-5-yl]nicotinamide;
N-{7-methoxy-8-[3-(3-methylmorpholin-4-yl)propoxy]-2,3-dihydroimidazo[1,2-c]quinazolin-5-yl}nicotinamide;
N-(8-{3-[2-(hydroxymethyl)morpholin-4-yl]propoxy}-7-methoxy-2,3-dihydroimidazo[1,2-c]quinazolin-5-yl)nicotinamide;
N-(8-{2-[4-(cyclobutylmethyl)morpholin-2-yl]ethoxy}-7-methoxy-2,3-dihydroimidazo[1,2-c]quinazolin-5-yl)nicotinamide;
N-(7-methoxy-8-{2-[4-(2-methoxyethyl)morpholin-2-yl]ethoxy}-2,3-dihydroimidazo[1,2-c]quinazolin-5-yl)nicotinamide;
N-{8-[(4-ethylmorpholin-2-yl)methoxy]-7-methoxy-2,3-dihydroimidazo[1,2-c]quinazolin-5-yl}nicotinamide;
N-(7-methoxy-8-{[4-(2-methoxyethyl)morpholin-2-yl]methoxy}-2,3-dihydroimidazo[1,2-c]quinazolin-5-yl)nicotinamide;
N-{7-methoxy-8-[(4-methylmorpholin-2-yl)methoxy]-2,3-dihydroimidazo[1,2-c]quinazolin-5-yl}nicotinamide;
N-[7-methoxy-8-(3-morpholin-4-ylpropoxy)-2,3-dihydroimidazo[1,2-c]quinazolin-5-yl]pyrimidine-4-carboxamide;
2-amino-N-[7-methoxy-8-(3-morpholin-4-ylpropoxy)-2,3-dihydroimidazo[1,2-c]quinazolin-5-yl]pyrimidine-4-carboxamide;
N-[7-methoxy-8-(3-morpholin-4-ylpropoxy)-2,3-dihydroimidazo[1,2-c]quinazolin-5-yl]-1-methyl-1H-imidazole-4-carboxamide;
rel-N-(8-{3-[(2R,6S)-2,6-dimethylmorpholin-4-yl]propoxy}-7-methoxy-2,3-dihydroimidazo[1,2-c]quinazolin-5-yl)pyrimidine-5-carboxamide;
rel-N-(8-{3-[(2R,6S)-2,6-dimethylmorpholin-4-yl]propoxy}-7-methoxy-2,3-dihydroimidazo[1,2-c]quinazolin-5-yl)-6-methylnicotinamide;
rel-6-acetamido-N-(8-{3-[(2R,6S)-2,6-dimethylmorpholin-4-yl]propoxy}-7-methoxy-2,3-dihydroimidazo[1,2-c]quinazolin-5-yl)nicotinamide;
N-[7-methoxy-8-(3-morpholin-4-ylpropoxy)-2,3-dihydroimidazo[1,2-c]quinazolin-5-yl]-1-methyl-1H-imidazole-5-carboxamide;
6-amino-N-[7-methoxy-8-(3-morpholin-4-ylpropoxy)-2,3-dihydroimidazo[1,2-c]quinazolin-5-yl]-2-methylnicotinamide;
2-amino-N-[7-methoxy-8-(3-morpholin-4-ylpropoxy)-2,3-dihydroimidazo[1,2-c]quinazolin-5-yl]-4-methylpyrimidine-5-carboxamide;
6-amino-5-bromo-N-[7-methoxy-8-(3-morpholin-4-ylpropoxy)-2,3-dihydroimidazo[1,2-c]quinazolin-5-yl]nicotinamide;
2-amino-N-[7-methoxy-8-(3-morpholin-4-ylpropoxy)-2,3-dihydroimidazo[1,2-c]quinazolin-5-yl]-1,3-oxazole-5-carboxamide;
N-[7-methoxy-8-(morpholin-2-ylmethoxy)-2,3-dihydroimidazo[1,2-c]quinazolin-5-yl]nicotinamide;
2-{[2-(dimethylamino)ethyl]amino}-N-{8-[3-(dimethylamino)propoxy]-7-methoxy-2,3-dihydroimidazo[1,2-c]quinazolin-5-yl}pyrimidine-5-carboxamide;
2-amino-N-{8-[3-(dimethylamino)propoxy]-7-methoxy-2,3-dihydroimidazo[1,2-c]quinazolin-5-yl}-1,3-thiazole-5-carboxamide;
rel-2-amino-N-(8-{3-[(2R,6S)-2,6-dimethylmorpholin-4-yl]propoxy}-7-methoxy-2,3-dihydroimidazo[1,2-c]quinazolin-5-yl)pyrimidine-5-carboxamide;
rel-6-amino-N-(8-{3-[(2R,6S)-2,6-dimethylmorpholin-4-yl]propoxy}-7-methoxy-2,3-dihydroimidazo[1,2-c]quinazolin-5-yl)nicotinamide;
2-[(2-hydroxyethyl)amino]-N-[7-methoxy-8-(3-morpholin-4-ylpropoxy)-2,3-dihydroimidazo[1,2-c]quinazolin-5-yl]pyrimidine-5-carboxamide;
N-[7-methoxy-8-(3-morpholin-4-ylpropoxy)-2,3-dihydroimidazo[1,2-c]quinazolin-5-yl]-2-[(3-methoxypropyl)amino]pyrimidine-5-carboxamide;
2-amino-N-{8-[3-(dimethylamino)propoxy]-7-methoxy-2,3-dihydroimidazo[1,2-c]quinazolin-5-yl}pyrimidine-5-carboxamide;
N-[7-methoxy-8-(3-morpholin-4-ylpropoxy)-2,3-dihydroimidazo[1,2-c]quinazolin-5-yl]-2-[(3-morpholin-4-ylpropyl)amino]pyrimidine-5-carboxamide;
2-[(2-methoxyethyl)amino]-N-[7-methoxy-8-(3-morpholin-4-ylpropoxy)-2,3-dihydroimidazo[1,2-c]quinazolin-5-yl]pyrimidine-5-carboxamide;
2-{[2-(dimethylamino)ethyl]amino}-N-[7-methoxy-8-(3-morpholin-4-ylpropoxy)-2,3-dihydroimidazo[1,2-c]quinazolin-5-yl]pyrimidine-5-carboxamide;
6-amino-N-{8-[3-(dimethylamino)propoxy]-7-methoxy-2,3-dihydroimidazo[1,2-c]quinazolin-5-yl}nicotinamide;
N-[7-methoxy-8-(3-morpholin-4-ylpropoxy)-2,3-dihydroimidazo[1,2-c]quinazolin-5-yl]-2-pyrrolidin-1-ylpyrimidine-5-carboxamide;
N-[7-methoxy-8-(3-morpholin-4-ylpropoxy)-2,3-dihydroimidazo[1,2-c]quinazolin-5-yl]-2-(4-methylpiperazin-1-yl)pyrimidine-5-carboxamide;
N-[7-methoxy-8-(3-morpholin-4-ylpropoxy)-2,3-dihydroimidazo[1,2-c]quinazolin-5-yl]-2-morpholin-4-ylpyrimidine-5-carboxamide;
N-[7-methoxy-8-(3-morpholin-4-ylpropoxy)-2,3-dihydroimidazo[1,2-c]quinazolin-5-yl]-6-piperazin-1-ylnicotinamide hydrochloride;
6-[(3S)-3-aminopyrrolidin-1-yl]-N-[7-methoxy-8-(3-morpholin-4-ylpropoxy)-2,3-dihydroimidazo[1,2-c]quinazolin-5-yl]nicotinamide hydrochloride hydrate;
6-[(3R)-3-aminopyrrolidin-1-yl]-N-[7-methoxy-8-(3-morpholin-4-ylpropoxy)-2,3-dihydroimidazo[1,2-c]quinazolin-5-yl]nicotinamide hydrochloride;
6-[(4-fluorobenzyl)amino]-N-[7-methoxy-8-(3-morpholin-4-ylpropoxy)-2,3-dihydroimidazo[1,2-c]quinazolin-5-yl]nicotinamide;
6-[(2-furylmethyl)amino]-N-[7-methoxy-8-(3-morpholin-4-ylpropoxy)-2,3-dihydroimidazo[1,2-c]quinazolin-5-yl]nicotinamide;
6-[(2-methoxyethyl)amino]-N-[7-methoxy-8-(3-morpholin-4-ylpropoxy)-2,3-dihydroimidazo[1,2-c]quinazolin-5-yl]nicotinamide;
N-[7-methoxy-8-(3-morpholin-4-ylpropoxy)-2,3-dihydroimidazo[1,2-c]quinazolin-5-yl]-6-(1H-pyrrol-1-yl)nicotinamide;
N-[7-methoxy-8-(3-morpholin-4-ylpropoxy)-2,3-dihydroimidazo[1,2-c]quinazolin-5-yl]-6-morpholin-4-ylnicotinamide;
N-{7-methoxy-8-[3-(methylamino)propoxy]-2,3-dihydroimidazo[1,2-c]quinazolin-5-yl}nicotinamide;
6-[(2,2-dimethylpropanoyl)amino]-N-[7-methoxy-8-(3-morpholin-4-ylpropoxy)-2,3-dihydroimidazo[1,2-c]quinazolin-5-yl]nicotinamide;
6-[(cyclopropylcarbonyl)amino]-N-[7-methoxy-8-(3-morpholin-4-ylpropoxy)-2,3-dihydroimidazo[1,2-c]quinazolin-5-yl]nicotinamide
N-[7-methoxy-8-(3-morpholin-4-ylpropoxy)-2,3-dihydroimidazo[1,2-c]quinazolin-5-yl]-6-(2,2,2-trifluoroethoxy)nicotinamide;
N-[7-methoxy-8-(3-morpholin-4-ylpropoxy)-2,3-dihydroimidazo[1,2-c]quinazolin-5-yl]-6-(trifluoromethyl)nicotinamide;
6-(isobutyrylamino)-N-[7-methoxy-8-(3-morpholin-4-ylpropoxy)-2,3-dihydroimidazo[1,2-c]quinazolin-5-yl]nicotinamide;
N-{7-methoxy-8-[3-(4-methylpiperazin-1-yl)propoxy]-2,3-dihydroimidazo[1,2-c]quinazolin-5-yl}nicotinamide;
N-[7-methoxy-8-(3-morpholin-4-ylpropoxy)-2,3-dihydroimidazo[1,2-c]quinazolin-5-yl]-2-{[(methylamino)carbonyl]amino}-1,3-thiazole-4-carboxamide;
N-[7-methoxy-8-(3-morpholin-4-ylpropoxy)-2,3-dihydroimidazo[1,2-c]quinazolin-5-yl]-6-{[(methylamino)carbonyl]amino}nicotinamide;
N-[7-methoxy-8-(3-morpholin-4-ylpropoxy)-2,3-dihydroimidazo[1,2-c]quinazolin-5-yl]-2-(methylamino)-1,3-thiazole-4-carboxamide;
N-[7-methoxy-8-(2-morpholin-4-ylethoxy)-2,3-dihydroimidazo[1,2-c]quinazolin-5-yl]nicotinamide;
N-{8-[2-(dimethylamino)ethoxy]-7-methoxy-2,3-dihydroimidazo[1,2-c]quinazolin-5-yl}-2,4-dimethyl-1,3-thiazole-5-carboxamide;
N-{8-[2-(dimethylamino)ethoxy]-7-methoxy-2,3-dihydroimidazo[1,2-c]quinazolin-5-yl}-6-methylnicotinamide;
6-{[(isopropylamino)carbonyl]amino}-N-[7-methoxy-8-(3-morpholin-4-ylpropoxy)-2,3-dihydroimidazo[1,2-c]quinazolin-5-yl]nicotinamide;
N-[7-methoxy-8-(3-morpholin-4-ylpropoxy)-2,3-dihydroimidazo[1,2-c]quinazolin-5-yl]-6-pyrrolidin-1-ylnicotinamide;
6-(dimethylamino)-N-[7-methoxy-8-(3-morpholin-4-ylpropoxy)-2,3-dihydroimidazo[1,2-c]quinazolin-5-yl]nicotinamide;
N-[7-methoxy-8-(3-piperidin-1-ylpropoxy)-2,3-dihydroimidazo[1,2-c]quinazolin-5-yl]nicotinamide;
N-[7-methoxy-8-(2-pyrrolidin-1-ylethoxy)-2,3-dihydroimidazo[1,2-c]quinazolin-5-yl]nicotinamide;
N-[7-methoxy-8-(2-piperidin-1-ylethoxy)-2,3-dihydroimidazo[1,2-c]quinazolin-5-yl]nicotinamide;
6-{[(ethylamino)carbonyl]amino}-N-[7-methoxy-8-(3-morpholin-4-ylpropoxy)-2,3-dihydroimidazo[1,2-c]quinazolin-5-yl]nicotinamide;
6-fluoro-N-[7-methoxy-8-(3-morpholin-4-ylpropoxy)-2,3-dihydroimidazo[1,2-c]quinazolin-5-yl]nicotinamide;
2-amino-N-[7-methoxy-8-(3-morpholin-4-ylpropoxy)-2,3-dihydroimidazo[1,2-c]quinazolin-5-yl]-1,3-oxazole-4-carboxamide;
2-(ethylamino)-N-[7-methoxy-8-(3-morpholin-4-ylpropoxy)-2,3-dihydroimidazo[1,2-c]quinazolin-5-yl]-1,3-thiazole-4-carboxamide;
N-[7-methoxy-8-(3-morpholin-4-ylpropoxy)-2,3-dihydroimidazo[1,2-c]quinazolin-5-yl]pyrazine-2-carboxamide;
N-[8-(2-aminoethoxy)-7-methoxy-2,3-dihydroimidazo[1,2-c]quinazolin-5-yl]nicotinamide;
6-amino-N-[7-methoxy-8-(3-morpholin-4-ylpropoxy)-2,3-dihydroimidazo[1,2-c]quinazolin-5-yl]nicotinamide;
N-[7-methoxy-8-(3-morpholin-4-ylpropoxy)-2,3-dihydroimidazo[1,2-c]quinazolin-5-yl]isonicotinamide;
N-{8-[3-(diethylamino)propoxy]-7-methoxy-2,3-dihydroimidazo[1,2-c]quinazolin-5-yl}nicotinamide;
N-{8-[2-(diisopropylamino)ethoxy]-7-methoxy-2,3-dihydroimidazo[1,2-c]quinazolin-5-yl}nicotinamide;
N-{8-[2-(diethylamino)ethoxy]-7-methoxy-2,3-dihydroimidazo[1,2-c]quinazolin-5-yl}nicotinamide;
N-{8-[3-(dimethylamino)propoxy]-7-methoxy-2,3-dihydroimidazo[1,2-c]quinazolin-5-yl}nicotinamide;
N-{8-[2-(dimethylamino)ethoxy]-7-methoxy-2,3-dihydroimidazo[1,2-c]quinazolin-5-yl}nicotinamide;
N-[7-methoxy-8-(3-morpholin-4-ylpropoxy)-2,3-dihydroimidazo[1,2-c]quinazolin-5-yl]-2-(methylamino)pyrimidine-5-carboxamide;
N-[7-methoxy-8-(3-morpholin-4-ylpropoxy)-2,3-dihydroimidazo[1,2-c]quinazolin-5-yl]-2-(methylthio)pyrimidine-5-carboxamide;
N-[8-(3-aminopropoxy)-7-methoxy-2,3-dihydroimidazo[1,2-c]quinazolin-5-yl]nicotinamide trifluoroacetate;
N-[7-methoxy-8-(3-morpholin-4-ylpropoxy)-2,3-dihydroimidazo[1,2-c]quinazolin-5-yl]thiophene-2-carboxamide;
N-[7-methoxy-8-(3-morpholin-4-ylpropoxy)-2,3-dihydroimidazo[1,2-c]quinazolin-5-yl]-2,4-dimethyl-1,3-thiazole-5-carboxamide;
2-methoxy-N-[7-methoxy-8-(3-morpholin-4-ylpropoxy)-2,3-dihydroimidazo[1,2-c]quinazolin-5-yl]pyrimidine-5-carboxamide;
N-[7-methoxy-8-(3-morpholin-4-ylpropoxy)-2,3-dihydroimidazo[1,2-c]quinazolin-5-yl]-3-furamide;
N-[7-methoxy-8-(3-morpholin-4-ylpropoxy)-2,3-dihydroimidazo[1,2-c]quinazolin-5-yl]thiophene-3-carboxamide;
N-[7-methoxy-8-(3-morpholin-4-ylpropoxy)-2,3-dihydroimidazo[1,2-c]quinazolin-5-yl]-2-methyl-1,3-thiazole-4-carboxamide;
6-methoxy-N-[7-methoxy-8-(3-morpholin-4-ylpropoxy)-2,3-dihydroimidazo[1,2-c]quinazolin-5-yl]nicotinamide;
5-methoxy-N-[7-methoxy-8-(3-morpholin-4-ylpropoxy)-2,3-dihydroimidazo[1,2-c]quinazolin-5-yl]nicotinamide;
N-[7-methoxy-8-(3-morpholin-4-ylpropoxy)-2,3-dihydroimidazo[1,2-c]quinazolin-5-yl]-6-methylnicotinamide;
6-(acetylamino)-N-[7-methoxy-8-(3-morpholin-4-ylpropoxy)-2,3-dihydroimidazo[1,2-c]quinazolin-5-yl]nicotinamide; and
N-[7-methoxy-8-(3-morpholin-4-ylpropoxy)-2,3-dihydroimidazo[1,2-c]quinazolin-5-yl]nicotinamide,
or a physiologically acceptable salt, solvate, hydrate or stereoisomer thereof.

8. The method according to claim 1, wherein said compound is 2-amino-N-[7-methoxy-8-(3-morpholin-4-ylpropoxy)-2,3-dihydroimidazo[1,2-c]quinazolin-5-yl]pyrimidine-5-carboxamide.

9. The method according to claim 1, wherein said compound is 2-amino-N-[7-methoxy-8-(3-morpholin-4-ylpropoxy)-2,3-dihydroimidazo[1,2-c]quinazolin-5-yl]pyrimidine-5-carboxamide dihydrochloride.

10. A combination of:
a) a compound of formula (I), 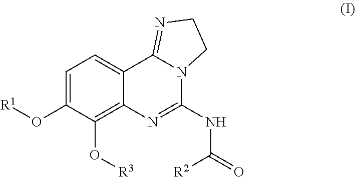
or a physiologically acceptable salt, solvate, hydrate or stereoisomer thereof, in which:
R¹ represents —(CH₂)ₙ—(CHR⁴)—(CH₂)ₘ—N(R⁵)(R^{5′});
R² represents a heteroaryl optionally substituted with 1, 2 or 3 R⁶ groups;
R³ represents alkyl or cycloalkyl;
R⁴ represents hydrogen, hydroxy or alkoxy;
R⁵ and R^{5′} may be the same or different and are independently, hydrogen, alkyl, cycloalkylalklyl, or alkoxyalkyl or R⁵ and R^{5′} may be taken together with the nitrogen atom to which they are bound to form a 3-7 membered nitrogen containing heterocyclic ring optionally containing at least one additional heteroatom selected from oxygen, nitrogen or sulfur and which may be optionally substituted with 1 or more R^{6′} groups, or R⁴ and R⁵ may be taken together with the atoms to which they are bound to form a 5-6 membered nitrogen containing heterocyclic ring optionally containing 1 or more nitrogen, oxygen or sulfur atoms and which may be optionally substituted with 1 or more R^{6′} groups;
each occurrence of R⁶ may be the same or different and is independently halogen, alkyl, alkenyl, alkynyl, cycloalkyl, cycloalkylalklyl, aryl, arylalkyl, heteroaryl, heteroarylalkyl, heterocyclic ring, heterocyclylalkyl, alkyl-OR⁷, alkyl-SR⁷, alkyl-N(R⁷)(R^{7′}), alkyl-COR⁷, —CN, —COOR⁷, —CON(R⁷)(R^{7′}), —OR⁷, —SR⁷, —N(R⁷)(R^{7′}), or —NR⁷COR⁷ each of which may be optionally substituted with 1 or more R⁸ groups;
each occurrence of R^{6′} may be the same or different and is independently alkyl, cycloalkylalklyl, or alkyl-OR⁷;
each occurrence of R⁷ and R^{7′} may be the same or different and is independently hydrogen, alkyl, alkenyl, alkynyl, cycloalkyl, cycloalkylalklyl, cycloalkenyl, aryl, arylalkyl, heteroaryl, heterocyclic ring, heterocyclylalkyl, or heteroarylalkyl;
each occurrence of R⁸ is independently nitro, hydroxy, cyano, formyl, acetyl, halogen, amino, alkyl, alkoxy, alkenyl, alkynyl, cycloalkyl, cycloalkylalklyl, cycloalkenyl, aryl, arylalkyl, heteroaryl, heterocyclic ring, heterocyclylalkyl, or heteroarylalkyl; and
n is an integer from 1-4 and m is an integer from 0-4 with the proviso that when when R⁴ and R⁵ are taken together with the atoms to which they are bound to form a 3-7 membered nitrogen containing ring, n+m≦4,
and
b) one or more further active agents.

11. The combination according to claim 10, wherein the compound of formula (I) is 2-amino-N-[7-methoxy-8-(3-morpholin-4-ylpropoxy)-2,3-dihydroimidazo[1,2-c]quinazolin-5- yl]pyrimidine-5-carboxamide.

12. The combination according to claim 10, wherein the compound of formula (I) is 2-amino-N-[7-methoxy-8-(3-morpholin-4-ylpropoxy)-2,3-dihydroimidazo[1,2-c]quinazolin-5-yl]pyrimidine-5-carboxamide dihydrochloride.

13. A pharmaceutical composition which comprises the combination according to claim 10.

14. A method for treatment or prophylaxis of endometrial cancer, comprising administering to a patient in need thereof a therapeutically effective amount of the combination according to claim 10.

15. A method for predicting the sensitivity and/or resistance of a patient with endometrial cancer to a compound of formula (I), 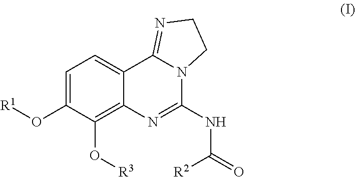
or a physiologically acceptable salt, solvate, hydrate or stereoisomer thereof, in which:
R¹ represents —(CH₂)ₙ—(CHR⁴)—(CH₂)ₘ—N(R⁵)(R^{5′});
R² represents a heteroaryl optionally substituted with 1, 2 or 3 R⁶ groups;
R³ represents alkyl or cycloalkyl;
R⁴ represents hydrogen, hydroxy or alkoxy;
R⁵ and R^{5′} may be the same or different and are independently, hydrogen, alkyl, cycloalkylalklyl, or alkoxyalkyl or R⁵ and R^{5′} may be taken together with the nitrogen atom to which they are bound to form a 3-7 membered nitrogen containing heterocyclic ring optionally containing at least one additional heteroatom selected from oxygen, nitrogen or sulfur and which may be optionally substituted with 1 or more R^{6′} groups, or R⁴ and R⁵ may be taken together with the atoms to which they are bound to form a 5-6 membered nitrogen containing heterocyclic ring optionally containing 1 or more nitrogen, oxygen or sulfur atoms and which may be optionally substituted with 1 or more R^{6′} groups;
each occurrence of R⁶ may be the same or different and is independently halogen, alkyl, alkenyl, alkynyl, cycloalkyl, cycloalkylalklyl, aryl, arylalkyl, heteroaryl, heteroarylalkyl, heterocyclic ring, heterocyclylalkyl, alkyl-OR⁷, alkyl-SR⁷, alkyl-N(R⁷)(R^{7′}), alkyl-COR⁷, —CN, —COOR⁷, —CON(R⁷)(R^{7′}), —OR⁷, —SR⁷, —N(R⁷)(R^{7′}), or —NR⁷COR⁷ each of which may be optionally substituted with 1 or more R⁸ groups;
each occurrence of R^{6′} may be the same or different and is independently alkyl, cycloalkylalklyl, or alkyl-OR⁷;
each occurrence of R⁷ and R^{7′} may be the same or different and is independently hydrogen, alkyl, alkenyl, alkynyl, cycloalkyl, cycloalkylalklyl, cycloalkenyl, aryl, arylalkyl, heteroaryl, heterocyclic ring, heterocyclylalkyl, or heteroarylalkyl;
each occurrence of R⁸ is independently nitro, hydroxy, cyano, formyl, acetyl, halogen, amino, alkyl, alkoxy, alkenyl, alkynyl, cycloalkyl, cycloalkylalklyl, cycloalkenyl, aryl, arylalkyl, heteroaryl, heterocyclic ring, heterocyclylalkyl, or heteroarylalkyl; and
n is an integer from 1-4 and m is an integer from 0-4 with the proviso that when when R⁴ and R⁵ are taken together with the atoms to which they are bound to form a 3-7 membered nitrogen containing ring, n+m≦4;
comprising identifying loss of tumor suppressor PTEN or FBXW7, either alone or in combination with another form of PI3K pathway activation selected from perturbation of any of the following alone or in combination: mutation in PIK3CA, PIK3CB, PIK3CD, PIK3CG, PIK3R1, PIK3R2, PIK3R3, PIK3R4, PIK3R5, FGFR1, FGFR2, FGFR3 and/or FGFR4; PTEN loss and alteration of PIK3CA, PIK3CB, PIK3CD, PIK3CG, PIK3R1, PIK3R2, PIK3R3, PIK3R4, PIK3R5, FGFR1, FGFR2, FGFR3 and/or FGFR4, which may be measured at either the protein level, mRNA level, or DNA level,
thus providing rationale-based dosage as defined herein to overcome said resistance of said patient with endometrial cancer to the compound of formula (I).

16. A method of determining the loss of tumor suppressor PTEN or FBXW7.

17. A method for determining perturbations in PIK3CA, PIK3CB, PIK3CD, PIK3CG, PIK3R1, PIK3R2, PIK3R3, PIK3R4,[[ ,]] PIK3R5, FGFR1, FGFR2, FGFR3 and/or FGFR4, PTEN loss and alteration of PIK3CA, PIK3CB, PIK3CD, PIK3CG, PIK3R1, PIK3R2, PIK3R3, PIK3R4, PIK3R5, FGFR1, FGFR2, FGFR3 and/or FGFR4.

18. The method of claim 1, wherein the endometrial cancer is selected from the group consisting of 1st line, 2nd line, relapsed, refractory, type I or type II endometrial cancer, and endometriosis.

19. The method of claim 7, wherein the compound of formula (I) is selected from the group consisting of:
N-[7-methoxy-8-(3-morpholin-4-ylpropoxy)-2,3-dihydroimidazo[1,2-c]quinazolin-5-yl]nicotinamide;
N-[7-methoxy-8-(3-morpholin-4-ylpropoxy)-2,3-dihydroimidazo[1,2-c]quinazolin-5-yl]-6-methylnicotinamide;
5-methoxy-N-[7-methoxy-8-(3-morpholin-4-ylpropoxy)-2,3-dihydroimidazo[1,2-c]quinazolin-5-yl]nicotinamide;
N-[7-methoxy-8-(3-morpholin-4-ylpropoxy)-2,3-dihydroimidazo[1,2-c]quinazolin-5-yl]-2,4-dimethyl-1,3-thiazole-5-carboxamide;
N-{8-[2-(dimethylamino)ethoxy]-7-methoxy-2,3-dihydroimidazo[1,2-c]quinazolin-5-yl}nicotinamide;
N-{8-[3-(dimethylamino)propoxy]-7-methoxy-2,3-dihydroimidazo[1,2-c]quinazolin-5-yl}nicotinamide;
6-{[(isopropylamino)carbonyl]amino}-N-[7-methoxy-8-(3-morpholin-4-ylpropoxy)-2,3-dihydroimidazo[1,2-c]quinazolin-5-yl]nicotinamide;
N-{8-[2-(dimethylamino)ethoxy]-7-methoxy-2,3-dihydroimidazo[1,2-c]quinazolin-5-yl}-2,4-dimethyl-1,3-thiazole-5-carboxamide;
N-[7-methoxy-8-(2-morpholin-4-ylethoxy)-2,3-dihydroimidazo[1,2-c]quinazolin-5-yl]nicotinamide;
rel-6-amino-N-(8-{3-[(2R,6S)-2,6-dimethylmorpholin-4-yl]propoxy}-7-methoxy-2,3-dihydroimidazo[1,2-c]quinazolin-5-yl)nicotinamide;
rel-2-amino-N-(8-{3-[(2R,6S)-2,6-dimethylmorpholin-4-yl]propoxy}-7-methoxy-2,3-dihydroimidazo[1,2-c]quinazolin-5-yl)pyrimidine-5-carboxamide;
2-amino-N-[7-methoxy-8-(3-morpholin-4-ylpropoxy)-2,3-dihydroimidazo[1,2-c]quinazolin-5-yl]pyrimidine-5-carboxamide;
N-{8-[2-(dimethylamino)ethoxy]-7-methoxy-2,3-dihydroimidazo[1,2-c]quinazolin-5-yl}pyrimidine-5-carboxamide; and
N-[7-methoxy-8-(3-morpholin-4-ylpropoxy)-2,3-dihydroimidazo[1,2-c]quinazolin-5-yl]pyrimidine-5-carboxamide,
or a physiologically acceptable salt, solvate, hydrate or stereoisomer thereof.

20. The method of claim 14, wherein the endometrial cancer is selected from the group consisting of 1st line, 2nd line, relapsed, refractory, type I or type II endometrial cancer, and endometriosis.

21. The method of claim 15, wherein the endometrial cancer is selected from the group consisting of 1st line, 2nd line, relapsed, refractory, type I or type II endometrial cancer, and endometriosis.

22. A method for treatment or prophylaxis of endometrial cancer, comprising administering to a patient in need thereof a therapeutically effective amount of a pharmaceutical composition according to claim 13.

23. The method of claim 22, wherein the endometrial cancer is selected from the group consisting of 1st line, 2nd line, relapsed, refractory, type I or type II endometrial cancer, and endometriosis.
